SEQUENCE LISTING (45 sequences)

SEQ ID NO: 1 - DNA, 11218 bp, Staphylococcus aureus
ttgattgaaaaaagtcaagcatgtcacgattcattgttagattctgtagggcaaacacctatggttcaacttcatcaactatttccgaaacatgaagtgtttgcaaagttagagtatatgaatcctggaggcagcatgaaagatcgacctgccaagtacatcattgaacatggtattaaacatggtttaatcactgagaatacacatttaattgaaagtacttctggtaatttaggcattgcgttggcaatgatagctaaaatcaagggattaaaactcacgtgtgttgttgatcctaaaatatcaccaacaaatttgaaaattattaaaagttatggtgccaatgtagaaatggttgaagaacctgatgcacatgggggttatttaatgactcgtattgcaaaggtgcaagaactgttagccactattgacgatgcatattggattaatcaatatgcgaatgagttaaattggcaatcccattatcatggtgcaggcacagagattgttgaaacaattaagcaacctatagattattttgtcgcgccagtcagcacgacaggtagcattatgggtatgagtagaaaaataaaagaagtgcatccaaacgcacaaattgttgctgttgatgcgaaagggtcagtcatttttggtgacaaacctattaatagagaattacctggtatcggtgctagtcgtgtacccgaaatattgaatagatcagaaattaatcaagtgatccatgtagatgattatcaatctgctttgggctgtcgaaaactgattgattatgaaggcatatttgccggaggttcaacaggttcgattattgcagcgattgagcagttgataacgtcaattgaagaaggtgcaacaattgtcacgattttaccagatcgaggcgatcgttacttagatttagtttattcagatacatggttagaaaaaatgaaatcaagacaaggagttaaatcagaatgaatagagagatgttgtatttaaatagatcagatattgaacaagcgggaggtaatcattcacaagtttatgtggacgcattaacagaagcattaacagcccatgcgcacaatgattttgtacaaccgcttaagccgtatttaagacaggatcctgaaaatggacacatcgcagatcgaattattgcaatgccaagtcatatcggtggtgaacacgcaatttcaggtattaagtggataggtagtaagcacgacaatccatcgaaacgtaatatggagcgtgcaagtggcgtcattattttgaatgatccagaaacgaattatccaattgcagttatggaagcaagtttaattagtagtatgcgtactgcagcagtttcagtgattgcagcaaagcatttggctaaaaaaggatttaaagacttaacaatcattggatgcgggctaatcggagacaagcaattacaaagtatgttagagcaattcgatcatattgaacgcgtgtttgtttacgatcaattctctgaagcatgtgcacgctttgttgatagatggcaacaacagcgtccggaaattaattttattgcgacagaaaatgctaaagaagcagtatcaaatggtgaagtagtcattacatgtaccgtaacggatcaaccatacattgaatatgattggttacaaaagggtgcatttattagcaacatttctatcatggatgtgcataaagaagtctttattaaagctgacaaagtcgtagtagatgactggtcacaatgtaatcgagaaaagaaaactattaaccaattggtgttagaaggtaaattcagcaaagaagctcttcatgctgaactaggacaacttgtgacaggtgacataccaggacgtgaagacgatgatgagatcatattacttaatccgatgggtatggctatcgaagatatttcaagtgcttattttatttatcaacaggcacaacaacaaaatattgggacaacattgaacctatattaagaatgcgaggtgtctgaacattgcagaatcatacagcagtcaatacagcacaagcgataatattaagagatttagttgatgcattattatttgaagatatagccggaattgtatcgaatagtgagattactaaagaaaacggacaaacgcttttgatatacgaacgtgaaacacaacaaataaagatacctgtttattttagtgctttaaatatgtttcgttacgaaagttcacaaccaattacgatagagggaagggtgtctaagcaacctttaacggcagctgaattttggcaaacaattgctaatatgaattgtgatctaagtcatgaatgggaagtggctcgcgttgaagaaggactgactactgctgccacacagcttgctaaacaattatcagaattagatttagcgtcacatccttttgtgatgtcagagcagtttgcaagtttaaaagatcgtccatttcatccattagctaaagaaaaaagaggattaagagaagcggattatcaagtgtatcaagctgaattaaatcaatcatttcctttaatggttgcagcagttaaaaagacacatatgattcatggcgatactgcaaatatcgatgaattagaaaatttgacagtacctataaaagaacaagcgacagacatgttaaatgatcaagggttatcaatagatgactatgtactatttccggtacatccttggcaatatcagcatattctgccgaacgtctttgcgaaagagattagtgaaaagttggttgtactattaccgttaaaatttggagattatctgtcgtcttcaagtatgcgttcattaattgatattggcgcaccgtataaccatgtcaaagtaccatttgcaatgcagtcattaggcgcattaaggctaacgcctacgcgttacatgaaaaacggagaacaagcagaacaattattacgtcagcttatagaaaaagatgaagcactagctaagtatgtcatggtttgtgatgaaacagcttggtggtcatatatgggtcaagataatgatattttcaaagatcaattaggtcatctaactgttcagctaagaaagtatcccgaagtgctagccaaaaatgatacgcaacagctagtgtcaatggcagcactcgcggcaaatgatcgcactttatatcaaatgatttgtggaaaagataatatttctaaaaatgatgtcatgacgttatttgaagatatcgcgcaagtctttttaaaggtaacactatcatttatgcaatacggcgcattaccagagttgcatggtcaaaatatattgttgtcatttgaagatggacgtgtacaaaaatgcgtgttacgtgatcatgatactgtcagaatttataaaccatggctaacagcacatcagctttcattgccgaagtatgtcgtcagagaagatacacctaatacgctaattaatgaggatttggaaacattctttgcttattttcaaacattagctgtatcggtaaatctatatgccattattgatgcaattcaagatttatttggtgtaagtgagcatgaacttatgtcgttgttaaaacaaattttaaaaaatgaagtggcaactatttcctgggttacaactgatcagctagctgtcagacacattttatttgataaacagacgtggccattcaaacaaattttattaccattgctatatcaacgtgatagtggtggaggtagtatgccttcaggtttaactaccgtaccaaatccaatggtgacatatgattaatcagtctatatggcgcagtaactttcgcattttatggctcagtcagtttatagcgattgctggactgacagtacttgtgccattattgccaatttatatggcatcactacaaaatctatcagtcgtagaaatacagttgtggagtggtatagcgattgctgctccagctgtaacgacgatgatagcttcgccgatatgggggaagctaggtgataagatcagccgaaaatggatggtgttaagagcgttacttggtttggcggtatgcttatttttaatggcattgtgtacgacaccattacagtttgtacttgtgaggttattgcagggactatttggtggtgttgttgatgcatcaagtgcgtttgcgagtgcagaggcgccagctgaagatcgtggaaaggtattaggaagactgcaaagttcagtcagcgcagggtctcttgtggggccattaattggcggtgttacagcttcgatattaggttttagtgcgttactgatgagtattgccgttattacttttattgtctgtattttcggtgcattaaaattgattgaaacgacacatatgccaaaatcacaaacaccaaatattaataaaggtattcgccgttcatttcaatgtctattatgcacacaacaaacatgtcgatttattatcgttggcgttttagcaaactttgctatgtatggcatgctaactgcattatcaccacttgcttcatcagtgaatcatacagcgatagatgaccgtagtgtgattggatttttacagtccgcattttggacggcttcgatattaagcgcgcctttatggggacgctttaatgataaatcatatgttaaatcagtatatatatttgccacgattgcatgtggttgtagtgcgatactgcaaggtttagcgacgaatatagagtttttaatggctgcaagaatacttcaaggattaacatatagtgcattgattcaaagtgtcatgtttgttgtcgtgaatgcgtgtcatcaacaacttaaaggcacatttgttggaacgacgaacagtatgttagttgttggtcaaattattggcagtcttagtggcgctgccattacaagttatactacaccagctactacgtttatcgttatgggcgtagtatttgcagtaagtagtttatttttaatttgttcaaccatcactaatcaaatcaacgatcacacattaatgaaattatgggagttgaaacaaaaaagtgcaaaataaagaattaatacaacatgcagcgtatgcggctatcgaacgcattttaaatgaatattttagagaagaaaatttatatcaagtaccacctcaaaatcatcaatggtctatacaattatcagagctcgaaactttaacgggtgaatttcgctattggtctgcgatggggcatcatatgtatcatccagaggtatggcttatcgatggaaaaagtaaaaaaataacaacttataaagaagcaattgcgcgtattttgcaacatatggctcaaagtgcagataatcaaacggcagtgcaacaacatatggcgcaaattatgtctgacatcgataatagcattcatcgcacggcacgttatttgcaaagtaacacaatagactacgtagaggatcgttatatcgtttcagaacaatctttatacttaggtcatccatttcatccgactcctaagagtgcaagtgggttttcagaagcagatttagagaaatatgcacccgaatgtcatacatcattccaattgcattatttagctgtgcatcaagatgttctgctcacgcgctatgtagaaggtaaagaagatcaggttgagaaagtgttgtatcaattagcagacatagatatatcagagatacccaaagattttattttattaccaacacatccttatcaaatcaatgtgttgcgacagcatccacagtatatgcaatatagtgaacaaggtttaataaaagaccttggcgtttccggtgattcagtgtacccgacgtcttcggttagaactgtattttcaaaagcattaaacatttatttaaaattaccgatacacgttaaaatcactaattttatacgtacgaatgaccttgaacagattgaacggacaattgatgccgcgcaagttatcgcatcagtcaaagatgaggttgaaacaccccattttaaattgatgtttgaagaaggatatcgtgcattgttaccgaatccattagggcaaacagttgaacctgaaatggatttattaacaaatagtgccatgattgttcgtgaagggataccgaattaccatgctgataaagatattcatgtattggcgtcattatttgaaacgatgcctgattcaccgatgtctaagttatcacaagtgattgagcaaagtggtttagcaccagaagcatggcttgaatgttatttgaatcgtacattattgccgatattaaagctgtttagtaacacaggcattagtctagaagcacatgtacaaaatacattaattgaattaaaagatggcatacccgacgtatgctttgtcagagatcttgaaggcatttgtctatctagaacgattgctactgaaaaacagcttgtgccaaatgttgtggcagcatcaagccctgttgtatatgcacatgatgaagcatggcatcgtcttaaatattacgttgtagtaaatcacttaggacatttagtatcaactattggtaaagcgactagaaatgaagttgtgttatggcaacttgtagcgcatcgtcttatgacttggaaaaaagaatacgcgaataacgcagtatttgttgactgtgtagaagatttatatcaaacgccgaccattgcggctaaagcgaatttgatgagtaaattgaatgattgtggtgcaaaccctatttatacacatataccaaatccaatttgtcataacaaggaggtatcgtattgtgaatcaaacaattcttaatcgtgtaaagactagagtgatgcaccaactggtatcatcacttatttatgagaatattgttgtgtataaagcgtcatatcaagacggtgtcggtcattttacaatagaaggacatgattcagagtatcgttttactgctgaaaagacacatagctttgatcgtatacgtatcacatcaccaattgagcgtgtcgtaggagatgaggcagatacaacaacagactatacacaattattgagagaggttgtatttacatttcctaaaaatgatgaaaagctagaacaatttattgtcgagttattacagacagaattaaaagatacgcaaagtatgcagtatcgagaatcaaacccaccagcaacacctgagacatttaacgactatgaattttatgcgatggaagggcatcagtatcatccaagttacaaatcacgtttaggatttacgttgagtgataatttgaaatttggtcctgattttgtaccaaacgttaaactgcagtggttagctatcgacaaagataaagtagaaacgacggtatcaagaaatgttgtagttaacgaaatgttacgtcaacaagttggcgataagacttatgaacattttgtacagcaaattgaagcatctggcaaacatgtaaatgatgttgagatgatacctgtacacccatggcagtttgaacatgtcatccaagttgatttggctgaagaaaggcttaatggcacagtactatggttaggggaaagtgatgagctatatcaccctcaacaatcgattcgtacgatgtcgccaatagacacgacaaaatattatttaaaggtaccaataagtataacgaacacttcaacgaaacgagtgttggcgcctcatacaattgaaaatgcagcgcaaattacggattggttaaagcagatacagcaacaagatatgtatttaaaagatgaattaaagacagtttttctaggggaagtcttaggacagtcttatttaaatacacaactttcgccttataaacaaactcaagtttatggtgcgttaggtgttatatggcgtgaaaatatatatcatatgttaatcgatgaagaggatgcgataccatttaatgcactttatgcaagtgataaggatggtgtaccattcattgaaaattggattaaacaatatggttctgaagcttggacaaagcaatttttagctgtagcgattcgtccaatgattcatatgctttattatcacggtattgcctttgaatcgcatgcacaaaatatgatgctcattcatgaaaatggttggcctacacgtattgccttaaaagatttccatgatggtgttcgttttaagcgtgagcatttaagtgaagctgcttcacacctgacattaaagccaatgccagaagcacataaaaaagtgaatagtaattcatttattgaaacagatgacgaacgtttagtacgcgactttttacatgatgcatttttctttattaatatcgccgaaatcatcttatttattgaaaagcaatatggtatcgatgaggagctgcaatggcaatgggttaaaggcatcatcgaggcgtatcaagaagcatttccagagttgaataactatcaacatttcgatttgtttgaacctacgattcaagttgaaaagttaacgacacgtcgattattaagtgactccgagttaagaattcatcatgttacaaatccattaggtgtaggaggtatcaatgatgcaacaactatctctgaaacatagattaaacaatggtgattcagtttatggcatttttaattctataccggacccattgatgatcgaggttatcgcagcaagcgggtatgactttgttgtgattgatacagaacacgtggcgattaatgatgagacactagcgcatttaattcgtgcagctgaagcagcgcatattataccaattgtacgtgtcactgcagtgatagatagagatatcattaaagtgttggatatgggtgcgagaggtattattgtgccacacgttaaagatcgtgagacagttgagcatattgtgaaattaagtcgttattacccgcaaggattaagaagtttgaatggtggtcgcatggcaagatttggacgtacaccattacttgatgcaatggagatggctaatgagcatattatggtgattgccatgatagaagatgttgaaggggttatggccattgacgatatagcacaagtcgaaggtttagacatgatagtcgaaggtgccgcagatttatcgcagtcacttggcataccatggcaaacgcgtgatgatcaagtaacatcacatgttcaacatatttttgaagttgtgaatgcacatggtaaacatttttgtgcattaccacgtgaagatgaagatattgcaaaatggcaggcacaaggtgtacaaacatttattttaggtgatgatcgcggaaaaatatatcgccatttaagtgcatctctagcgacgtctaaacagaaaggggatgaaggctaatgcgtatagttcaacctgttattgaacaattaaaagcacaatctcatccagtttgtcattatatctatgatttagtcggactggaacatcatttgcaacatattacatcgtcattgccgagtaattgtcaaatgtactatgcaatgaaagcaaatagtgaacgaaaaatcctagatacaattagtcagtatgttgaaggattcgaagttgcatctcaaggtgaaatagcaaaaggtcttgcttttaaaccagcaaatcatattatttttggtggccctggtaagacagacgaggaactaagatatgcagtaagtgaaggtgttcagcgtattcatgttgaaagtatgcatgaattacaacggctaaatgccatcttagaagatgaagataagacacaacacattttattgcgtgttaatttagcaggaccatttcccaatgcaacgttgcatatggcaggacgcccaacacaatttggtatttctgaagacgaagttgatgatgtcattgaagctgcgctcgcaatgccaaagattcatctagatggatttcattttcattctatttctaacaatttagactcgaatttacatgtcgatgtagtgaaactttattttaaaaaagcaaaggcatggtctgaaaaacatcgatttccactcaaacatatcaatcttggtggtggcataggcgttaactatgcagatttaactaaccaatttgaatgggataattttgtagaacgttttaaaacacttatcgttgagcaagaaatggaagatgtgacattgaactttgaatgtgggcgctttattgtggcacatattggttactatgtgacagaagtgctagacattaagaaagtacatggtgcttggtatgccatattaagaggaggtacgcaacaatttagactgccggtatcttggcaacataaccatccttttgacatttatcgctataaggacaatccatattcatttgaaaaagtttcaatttcgagacaggacacaacgttagtcggtcaattatgtacaccgaaagatgtctttgctagagaagtacagatagacgcaatcagtacaggcgacgttattgttttcaaatatgcaggtgcatacggatggtctatttcacatcacgatttcttaagccatccacatcctgaatttatttatttaacacaaacaaaggaggatgaataactattgaatcatattcatgaacatttaaaattggtaccagtagataagattgatcttcacgaaacattcgaacctttaagattggaaaaaacgaaaagtagtattgaagcagatgattttatacgtcatcctattttagtgacagcgatgcaacatggtagatatatggttatagatggtgtgcatcggtatacaagtttgaaagcgttaggatgtaagaaagttccagtgcaagaaatccatgaaacacaatattcaattagtacatggcaacataaagttccatttggtgtgtggtgggaaacgttacaacaagaacatcgcttgccatggactactgagacaagacaagaagcgccatttattacaatgtgtcatggtgatacagaacaatatttgtatacaaaagatttaggcgaagcacattttcaagtatgggaaaaggttgtcgcaagttatagtggttgttgttctgtagagagaattgcacaaggtacatatccttgtctttctcaacaagatgtactcatgaagtatcagccattgagttataaggaaattgaagcggttgttcataaaggggaaactgtgccagcaggtgtgacacgctttaatatttcaggacgatgtcttaatcttcaagtaccactggcattacttaaacaagatgatgatgttgaacaactgcgcaattggaagcagtttttagcagataagtttgccaatatgagatgctatactgaaaaagtatacttggtggagcaatag

SEQ ID NO: 2 - DNA, 978 bp, Staphylococcus aureus
ttgattgaaaaaagtcaagcatgtcacgattcattgttagattctgtagggcaaacacctatggttcaacttcatcaactatttccgaaacatgaagtgtttgcaaagttagagtatatgaatcctggaggcagcatgaaagatcgacctgccaagtacatcattgaacatggtattaaacatggtttaatcactgagaatacacatttaattgaaagtacttctggtaatttaggcattgcgttggcaatgatagctaaaatcaagggattaaaactcacgtgtgttgttgatcctaaaatatcaccaacaaatttgaaaattattaaaagttatggtgccaatgtagaaatggttgaagaacctgatgcacatgggggttatttaatgactcgtattgcaaaggtgcaagaactgttagccactattgacgatgcatattggattaatcaatatgcgaatgagttaaattggcaatcccattatcatggtgcaggcacagagattgttgaaacaattaagcaacctatagattattttgtcgcgccagtcagcacgacaggtagcattatgggtatgagtagaaaaataaaagaagtgcatccaaacgcacaaattgttgctgttgatgcgaaagggtcagtcatttttggtgacaaacctattaatagagaattacctggtatcggtgctagtcgtgtacccgaaatattgaatagatcagaaattaatcaagtgatccatgtagatgattatcaatctgctttgggctgtcgaaaactgattgattatgaaggcatatttgccggaggttcaacaggttcgattattgcagcgattgagcagttgataacgtcaattgaagaaggtgcaacaattgtcacgattttaccagatcgaggcgatcgttacttagatttagtttattcagatacatggttagaaaaaatgaaatcaagacaaggagttaaatcagaa

SEQ ID NO: 3 - DNA, 978 bp, Staphylococcus aureus
ttctgatttaactccttgtcttgatttcattttttctaaccatgtatctgaataaactaaatctaagtaacgatcgcctcgatctggtaaaatcgtgacaattgttgcaccttcttcaattgacgttatcaactgctcaatcgctgcaataatcgagcctgttgaacctccggcaaatatgccttcataatcaatcagttttcgacagcccaaagcagattgataatcatctacatggatcacttgattaatttctgatctattcaatatttcgggtacacgactagcaccgataccaggtaattctctattaataggtttgtcaccaaaaatgactgaccctttcgcatcaacagcaacaatttgtgcgtttggatgaccttcttttatttttctactcatacccataatgctacctgtcgtgctgactggcgcgacaaaataatctataggttgcttaattgtttcaacaatctctgtgcctgcaccatgataatgggattgccaatttaactcattcgcatattgattaatccaatatgcatcgtcaatagtggctaacagttcttgcacctttgcaatacgagtcattaaataacccccatgtgcatcaggttcttcaaccatttctacattggcaccataacttttaataattttcaaatttgttggtgatattttaggatcaacaacacacgtgagttttaatcccttgattttagctatcattgccaacgcaatgcctaaattaccagaagtactttcaattaaatgtgtattctcagtgattaaaccatgtttaataccatgttcaatgatgtacttggcaggtcgatctttcatgctgcctccaggattcatatactctaactttgcaaacacttcatgtttcggaaatagttgatgaagttgaaccataggtgtttgccctacagaatctaacaatgaatcgtgacatgcttgacttttttcaatcaa

SEQ ID NO: 4 - protein, 326 aa, Staphylococcus aureus
MIEKSQACHDSLLDSVGQTPMVQLHQLFPKHEVFAKLEYMNPGGSMKDRPAKYIIEHGIKHGLITENTHLIESTSGNLGIALAMIAKIKGLKLTCVVDPKISPTNLKIIKSYGANVEMVEEPDAHGGYLMTRIAKVQELLATIDDAYWINQYANELNWQSHYHGAGTEIVETIKQPIDYFVAPVSTTGSIMGMSRKIKEVHPNAQIVAVDAKGSVIFGDKPINRELPGIGASRVPEILNRSEINQVIHVDDYQSALGCRKLIDYEGIFAGGSTGSIIAAIEQLITSIEEGATIVTILPDRGDRYLDLVYSDTWLEKMKSRQGVKSE

SEQ ID NO: 5 - DNA, 1008 bp, Staphylococcus aureus
atgaatagagagatgttgtatttaaatagatcagatattgaacaagcgggaggtaatcattcacaagtttatgtggacgcattaacagaagcattaacagcccatgcgcacaatgattttgtacaaccgcttaagccgtatttaagacaggatcctgaaaatggacacatcgcagatcgaattattgcaatgccaagtcatatcggtggtgaacacgcaatttcaggtattaagtggataggtagtaagcacgacaatccatcgaaacgtaatatggagcgtgcaagtggtgtcattattttgaatgatccagaaacgaattatccaattgcagttatggaagcaagtttaattagtagtatgcgtactgcagcagtttcagtgattgcagctaagcatttggctaaaaaaggatttaaagacttaacaatcattggttgcgggctaatcggagacaagcaattacaaagtatgttagagcaattcgatcatattaaacgcgtgtttgtttacgatcaattctctgaagcatgtgcacgctttgttgatagatggcaacaacagcgtccggaaattaattttattgcgacagaaaatgctaaagaagcagtatcaaatggtgaagtagtcattacatgtaccgtaacggatcaaccatacattgaatatgattggttacaaaagggtgcatttattagcaacatttctatcatggatgtgcataaagaagtctttattaaagctgacaaagtcgtagtagatgactggtcacaatgtaatcgagaaaagaaaactattaaccaattggtgttagaaggtaaattcagcaaagaagcacttcatgctgaactagggcaacttgtgacaggtgacataccaggacgtgaagacgatgatgaaatcatattacttaatccgatgggtatggctatcgaagatatttcaagtgcttattttatttatcaacaggcacaacaacaaaatattgggacaacattgaacctatat

SEQ ID NO: 6 - DNA, 1008 bp, Staphylococcus aureus
atataggttcaatgttgtcccaatattttgttgttgtgcctgttgataaataaaataagcacttgaaatatcttcgatagccatacccatcggattaagtaatatgatttcatcatcgtcttcacgtcctggtatgtcacctgtcacaagttgccctagttcagcatgaagtgcttctttgctgaatttaccttctaacaccaattggttaatagttttcttttctcgattacattgtgaccagtcatctactacgactttgtcagctttaataaagacttctttatgcacatccatgatagaaatgttgctaataaatgcacccttttgtaaccaatcatattcaatgtatggttgatccgttacggtacatgtaatgactacttcaccatttgatactgcttctttagcattttctgtcgcaataaaattaatttccggacgctgttgttgccatctatcaacaaagcgtgcacatgcttcagagaattgatcgtaaacaaacacgcgtttaatatgatcgaattgctctaacatactttgtaattgcttgtctccgattagcccgcaaccaatgattgttaagtctttaaatccttttttagccaaatgcttagctgcaatcactgaaactgctgcagtacgcatactactaattaaacttgcttccataactgcaattggataattcgtttctggatcattcaaaataatgacaccacttgcacgctccatattacgtttcgatggattgtcgtgcttactacctatccacttaatacctgaaattgcgtgttcaccaccgatatgacttggcattgcaataattcgatctgcgatgtgtccattttcaggatcctgtcttaaatacggcttaagcggttgtacaaaatcattgtgcgcatgggctgttaatgcttctgttaatgcgtccacataaacttgtgaatgattacctcccgcttgttcaatatctgatctatttaaatacaacatctctctattcat

SEQ ID NO: 7 - protein, 336 aa, Staphylococcus aureus
MNREMLYLNRSDIEQAGGNHSQVYVDALTEALTAHAHNDFVQPLKPYLRQDPENGHIADRIIAMPSHIGGEHAISGIKWIGSKHDNPSKRNMERASGVIILNDPETNYPIAVMEASLISSMRTAAVSVIAAKHLAKKGFKDLTIIGCGLIGDKQLQSMLEQFDHIERVFVYDQFSEACARFVDRWQQQRPEINFIATENAKEAVSNGEVVITCTVTDQPYIEYDWLQKGAFISNISIMDVHKEVFIKADKVVVDDWSQCNREKKTINQLVLEGKFSKEALHAELGQLVTGDIPGREDDDEIILLNPMGMAIEDISSAYFIYQQAQQQNIGTTLNLY

SEQ ID NO: 8 - DNA, 1752 bp, Staphylococcus aureus
ttgcagaatcatacagcagtcaatacagcacaagcgataatattaagagatttagttgatgcattattatttgaagatatagccggaattgtatcgaatagtgagattactaaagaaaacggacaaacgcttttgatatacgaacgtgaaacacaacaaataaagatacctgtttattttagtgctttaaatatgtttcgttacgaaagttcacaaccaattacgatagagggaagggtgtctaagcaacctttaacggcagctgaattttggcaaacaattgctaatatgaattgtgatctaagtcatgaatgggaagtggctcgcgttgaagaaggactgactactgctgccacacagcttgctaaacaattatcagaattagatttagcgtcacatccttttgtgatgtcagagcagtttgcaagtttaaaagatcgtccatttcatccattagctaaagaaaaaagaggattaagagaagcggattatcaagtgtatcaagctgaattaaatcaatcatttcctttaatggttgcagcagttaaaaagacacatatgattcatggcgatactgcaaatatcgatgaattagaaaatttgacagtacctataaaagaacaagcgacagacatgttaaatgatcaagggttatcaatagatgactatgtactatttccggtacatccttggcaatatcagcatattctgccgaacgtctttgcgacagagattagtgaaaagttggttgtactattaccgttaaaatttggagattatctgtcgtcttcaagtatgcgttcattaattgatattggcgcaccgtataaccatgtcaaagtaccatttgcaatgcagtcattaggcgcattaaggctaacgcctacgcgttacatgaaaaacggagaacaagcagaacaattattacgtcagcttatagaaaaagatgaagcactagctaagtatgtcatggtttgtgatgaaacagcttggtggtcatatatgggtcaagataatgatattttcaaagatcaattaggtcatctaactgttcagctaagaaagtatcccgaagtgctagccaaaaatgatacgcaacagctagtgtcaatggcagcactcgcggcaaatgatcgcactttatatcaaatgatttgtggaaaagataatatttctaaaaatgatgtcatgacgttatttgaagatatcgcgcaagtctttttaaaggtaacactatcatttatgcaatacggcgcattaccagagttgcatggtcaaaatatattgttgtcatttgaagatggacgtgtacaaaaatgcgtgttacgtgatcatgatactgtcagaatttataaaccatggctaacagcacatcagctttcattgccgaagtatgtcgtcagagaagatacacctaatacgctaattaatgaggatttggaaacattctttgcttattttcaaacattagctgtatcggtaaatctatatgccattattgatgcaattcaagatttatttggtgtaagtgagcatgaacttatgtcgttgttaaaacaaattttaaaaaatgaagtggcaactatttcctgggttacaactgatcagctagctgtcagacacattttatttgataaacagacgtggccattcaaacaaattttattaccattgctatatcaacgtgatagtggtggaggtagtatgccttcaggtttaactaccgtaccaaatccaatggtgacatatgat

SEQ ID NO: 9 - DNA, 1752 bp, Staphylococcus aureus
atcatatgtcaccattggatttggtacggtagttaaacctgaaggcatactacctccaccactatcacgttgatatagcaatggtaataaaatttgtttgaatggccacgtctgtttatcaaataaaatgtgtctgacagctagctgatcagttgtaacccaggaaatagttgccacttcattttttaaaatttgttttaacaacgacataagttcatgctcacttacaccaaataaatcttgaattgcatcaataatggcatatagatttaccgatacagctaatgtttgaaaataagcaaagaatgtttccaaatcctcattaattagcgtattaggtgtatcttctctgacgacatacttcggcaatgaaagctgatgtgctgttagccatggtttataaattctgacagtatcatgatcacgtaacacgcatttttgtacacgtccatcttcaaatgacaacaatatattttgaccatgcaactctggtaatgcgccgtattgcataaatgatagtgttacctttaaaaagacttgcgcgatatcttcaaataacgtcatgacatcatttttagaaatattatcttttccacaaatcatttgatataaagtgcgatcatttgccgcgagtgctgccattgacactagctgttgcgtatcatttttggctagcacttcgggatactttcttagctgaacagttagatgacctaattgatctttgaaaatatcattatcttgacccatatatgaccaccaagctgtttcatcacaaaccatgacatacttagctagtgcttcatctttttctataagctgacgtaataattgttctgcttgttctccgtttttcatgtaacgcgtaggcgttagccttaatgcgcctaatgactgcattgcaaatggtactttgacatggttatacggtgcgccaatatcaattaatgaacgcatacttgaagacgacagataatctccaaattttaacggtaatagtacaaccaacttttcactaatctctgtcgcaaagacgttcggcagaatatgctgatattgccaaggatgtaccggaaatagtacatagtcatctattgataacccttgatcatttaacatgtctgtcgcttgttcttttataggtactgtcaaattttctaattcatcgatatttgcagtatcgccatgaatcatatgtgtctttttaactgctgcaaccattaaaggaaatgattgatttaattcagcttgatacacttgataatccgcttctcttaatcctcttttttctttagctaatggatgaaatggacgatcttttaaacttgcaaactgctctgacatcacaaaaggatgtgacgctaaatctaattctgataattgtttagcaagctgtgtggcagcagtagtcagtccttcttcaacgcgagccacttcccattcatgacttagatcacaattcatattagcaattgtttgccaaaattcagctgccgttaaaggttgcttagacacccttccctctatcgtaattggttgtgaactttcgtaacgaaacatatttaaagcactaaaataaacaggtatctttatttgttgtgtttcacgttcgtatatcaaaagcgtttgtccgttttctttagtaatctcactattcgatacaattccggctatatcttcaaataataatgcatcaactaaatctcttaatattatcgcttgtgctgtattgactgctgtatgattctgcaa

SEQ ID NO: 10 - protein, 584 aa, Staphylococcus aureus
MQNHTAVNTAQAIILRDLVDALLFEDIAGIVSNSEITKENGQTLLIYERETQQIKIPVYFSALNMFRYESSQPITIEGRVSKQPLTAAEFWQTIANMNCDLSHEWEVARVEEGLTTAATQLAKQLSELDLASHPFVMSEQFASLKDRPFHPLAKEKRGLREADYQVYQAELNQSFPLMVAAVKKTHMIHGDTANIDELENLTVPIKEQATDMLNDQGLSIDDYVLFPVHPWQYQHILPNVFAKEISEKLVVLLPLKFGDYLSSSSMRSLIDIGAPYNHVKVPFAMQSLGALRLTPTRYMKNGEQAEQLLRQLIEKDEALAKYVMVCDETAWWSYMGQDNDIFKDQLGHLTVQLRKYPEVLAKNDTQQLVSMAALAANDRTLYQMICGKDNISKNDVMTLFEDIAQVFLKVTLSFMQYGALPELHGQNILLSFEDGRVQKCVLRDHDTVRIYKPWLTAHQLSLPKYVVREDTPNTLINEDLETFFAYFQTLAVSVNLYAIIDAIQDLFGVSEHELMSLLKQILKNEVATISWVTTDQLAVRHILFDKQTWPFKQILLPLLYQRDSGGGSMPSGLTTVPNPMVTYD

SEQ ID NO: 11 - DNA, 1254 bp, Staphylococcus aureus
atgattaatcagtctatatggcgcagtaactttcgcattttatggctcagtcagtttatagcgattgctggactgacagtacttgtgccattattgccaatttatatggcatcactacaaaatctatcagtcgtagaaatacagttgtggagtggtatagcgattgctgctccagctgtaacgacgatgatagcttcgccgatatgggggaagctaggtgataagatcagccgaaaatggatggtcttaagagcgttacttggtttggcggtatgcttatttttaatggcattgtgtacgacaccattacagtttgtacttgtgaggttattgcagggactatttggtggtgttgttgatgcatcaagtgcgtttgcgagtgcagaggcgccagctgaagatcgtggaaaggtattaggaagactgcaaagttcagtcagcgcagggtctcttgtggggccattaattggcggtgttacagcttcgatattaggttttagtgcgttactgatgagtattgccgttattacttttattgtctgtattttcggtgcattaaaattgattgaaacgacacatatgccaaaatcacaaacaccaaatattaataaaggtattcgccgttcatttcaatgtctattatgcacacaacaaacatgtcgatttattatcgttggcgttttagcaaactttgctatgtatggcatgctaactgcattatcaccacttgcttcatcagtgaatcatacagcgatagatgaccgtagtgtgattggatttttacagtccgcattttggacggcttcgatattaagcgcgcctttatggggacgctttaatgataaatcatatgttaaatcagtatatatatttgccacgattgcatgtggttgtagtgcgatactgcaaggtttagcgacgaatatagagtttttaatggctgcaagaatacttcaaggattaacatatagtgcattgattcaaagtgtcatgtttgttgtcgtgaatgcgtgtcatcaacaacttaaaggcacatttgttggaacgacgaacagtatgttagttgttggtcaaattattggcagtcttagtggcgctgccattacaagttatactacaccagctactacgtttatcgttatgggcgtagtatttgcagtaagtagtttatttttaatttgttcaaccatcactaatcaaatcaacgatcacacattaatgaaattatgggagttgaaacaaaaaagtgcaaaa

SEQ ID NO: 12 - DNA, 1254 bp, Staphylococcus aureus
ttttgcacttttttgtttcaactcccataatttcattaatgtgtgatcgttgatttgattagtgatggttgaacaaattaaaaataaactacttactgcaaatactacgcccataacgataaacgtagtagctggtgtagtataacttgtaatggcagcgccactaagactgccaataatttgaccaacaactaacatactgttcgtcgttccaacaaatgtgcctttaagttgttgatgacacgcattcacgacaacaaacatgacactttgaatcaatgcactatatgttaatccttgaagtattcttgcagccattaaaaactctatattcgtcgctaaaccttgcagtatcgcactacaaccacatgcaatcgtggcaaatatatatactgatttaacatatgatttatcattaaagcgtccccataaaggcgcgcttaatatcgaagccgtccaaaatgcggactgtaaaaatccaatcacactacggtcatctatcgctgtatgattcactgatgaagcaagtggtgataatgcagttagcatgccatacatagcaaagtttgctaaaacgccaacgataataaatcgacatgtttgttgtgtgcataatagacattgaaatgaacggcgaatacctttattaatatttggtgtttgtgattttggcatatgtgtcgtttcaatcaattttaatgcaccgaaaatacagacaataaaagtaataacggcaatactcatcagtaacgcactaaaacctaatatcgaagctgtaacaccgccaattaatggccccacaagagaccctgcgctgactgaactttgcagtcttcctaatacctttccacgatcttcagctggcgcctctgcactcgcaaacgcacttgatgcatcaacaacaccaccaaatagtccctgcaataacctcacaagtacaaactgtaatggtgtcgtacacaatgccattaaaaataagcataccgccaaaccaagtaacgctcttaagaccatccattttcggctgatcttatcacctagcttcccccatatcggcgaagctatcatcgtcgttacagctggagcagcaatcgctataccactccacaactgtatttctacgactgatagattttgtagtgatgccatataaattggcaataatggcacaagtactgtcagtccagcaatcgctataaactgactgagccataaaatgcgaaagttactgcgccatatagactgattaatcat

SEQ ID NO: 13 - protein, 418 aa, Staphylococcus aureus
MINQSIWRSNFRILWLSQFIAIAGLTVLVPLLPIYMASLQNLSVVEIQLWSGIAIAAPAVTTMIASPIWGKLGDKISRKWMVLRALLGLAVCLFLMALCTTPLQFVLVRLLQGLFGGVVDASSAFASAEAPAEDRGKVLGRLQSSVSAGSLVGPLIGGVTASILGFSALLMSIAVITFIVCIFGALKLIETTHMPKSQTPNINKGIRRSFQCLLCTQQTCRFIIVGVLANFAMYGMLTALSPLASSVNHTAIDDRSVIGFLQSAFWTASILSAPLWGRFNDKSYVKSVYIFATIACGCSAILQGLATNIEFLMAARILQGLTYSALIQSVMFVVVNACHQQLKGTFVGTTNSMLVVGQIIGSLSGAAITSYTTPATTFIVMGVVFAVSSLFLICSTITNQINDHTLMKLWELKQKSAK

SEQ ID NO: 14 - DNA, 1734 bp, Staphylococcus aureus
gtgcaaaataaagaattaatacaacatgcagcgtatgcggctatcgaacgcattttaaatgaatattttagagaagaaaatttatatcaagtaccacctcaaaatcatcaatggtctatacaattatcagagctcgaaactttaacgggtgaatttcgctattggtctgcgatggggcatcatatgtatcatccagaggtatggcttatcgatggaaaaagtaaaaaaataacaacttataaagaagcaattgcgcgtattttgcaacatatggctcaaagtgcagataatcaaacggcagtgcaacaacatatggcgcaaattatgtctgacatcgataatagcattcatcgcacggcgcgttatttgcaaagtaacacaatagactacgtagaggatcgttatatcgtttcagaacaatctttatacttaggtcatccatttcatccgactcctaagagtgcaagtgggttttcagaagcagatttagagaaatatgcacccgaatgtcatacatcattccaattgcattatttagctgtgcatcaagatgttctgctcacgcgctatgtagaaggtaaagaagatcaggttgagaaagtgttgtatcaattagcagacatagatatatcagagatacccaaagattttattttattaccaacacatccttatcaaatcaatgtgttgcgacagcatccacagtatatgcaatatagtgaacaaggtttaataaaagaccttggcgtttccggtgatttagtgtacccgacgtcttcggttagaactgtattttcaaaagcattaaacatttatttaaaattaccgatacacgttaaaattactaattttatacgtacgaatgatcttgaacaaattgaacggacaattgatgccgcgcaagttatcgcatcagtcaaagatgaggttgaaacaccccattttaaattgatgtttgaagaaggatatcgtgcattgttaccgaatccattagggcaaacagttgaacctgaaatggatttattaacaaatagtgccatgattgttcgtgaagggataccgaattaccatgctgataaagatattcatgtattggcgtcattatttgaaacgatgcctgattcaccgatctctaagttagcacaagtgattgagcaaagtggtttagcaccagaagcatggcttgaatgttatttggatcgtacattattgccgatattaaagttgtttagtaacacaggcattagtctagaagcacatgtacaaaatacattaattgaattaaaagatggcatacccgacgtatgctttgtcagagatcttgaaggcatttgtctatctagaacgattgctactgaaaaacagcttgtgccaaatgttgtggcagcatcaagccctgttgtatatgcacatgatgaagcatggcatcgtcttaaatattatgttgtagtaaatcacttaggacatttagtatcaactattggtaaagcaactagaaatgaagttgtgttatggcaacttgtagcgcatcgtcttatgacttggaaaaaagaatacgcgaataacgcagtatttgttgactgtgtagaagatttatatcaaacgccgaccattgcggctaaagcgaatttgatgagtaaattgaatgattgtggtgcaaaccctatttatacacatataccaaatccaatttgttataacaaggaggtatcgtattgtgaatcaaacaattct

SEQ ID NO: 15 - DNA, 1734 bp, Staphylococcus aureus
agaattgtttgattcacaatacgatacctccttgttataacaaattggatttggtatatgtgtataaatagggtttgcaccacaatcattcaatttactcatcaaattcgctttagccgcaatggtcggcgtttgatataaatcttctacacagtcaacaaatactgcgttattcgcgtattcttttttccaagtcataagacgatgcgctacaagttgccataacacaacttcatttctagttgctttaccaatagttgatactaaatgtcctaagtgatttactacaacataatatttaagacgatgccatgcttcatcatgtgcatatacaacagggcttgatgctgccacaacatttggcacaagctgtttttcagtagcaatcgttctagatagacaaatgccttcaagatctctgacaaagcatacgtcgggtatgccatcttttaattcaattaatgtattttgtacatgtgcttctagactaatgcctgtgttactaaacaactttaatatcggcaataatgtacgatccaaataacattcaagccatgcttctggtgctaaaccactttgctcaatcacttgtgctaacttagagatcggtgaatcaggcatcgtttcaaataatgacgccaatacatgaatatctttatcagcatggtaattcggtatcccttcacgaacaatcatggcactatttgttaataaatccatttcaggttcaactgtttgccctaatggattcggtaacaatgcacgatatccttcttcaaacatcaatttaaaatggggtgtttcaacctcatctttgactgatgcgataacttgcgcggcatcaattgtccgttcaatttgttcaagatcattcgtacgtataaaattagtaattttaacgtgtatcggtaattttaaataaatgtttaatgcttttgaaaatacagttctaaccgaagacgtcgggtacactaaatcaccggaaacgccaaggtcttttattaaaccttgttcactatattgcatatactgtggatgctgtcgcaacacattgatttgataaggatgtgttggtaataaaataaaatctttgggtatctctgatatatctatgtctgctaattgatacaacactttctcaacctgatcttctttaccttctacatagcgcgtgagcagaacatcttgatgcacagctaaataatgcaattggaatgatgtatgacattcgggtgcatatttctctaaatctgcttctgaaaacccacttgcactcttaggagtcggatgaaatggatgacctaagtataaagattgttctgaaacgatataacgatcctctacgtagtctattgtgttactttgcaaataacgcgccgtgcgatgaatgctattatcgatgtcagacataatttgcgccatatgttgttgcactgccgtttgattatctgcactttgagccatatgttgcaaaatacgcgcaattgcttctttataagttgttatttttttactttttccatcgataagccatacctctggatgatacatatgatgccccatcgcagaccaatagcgaaattcacccgttaaagtttcgagctctgataattgtatagaccattgatgattttgaggtggtacttgatataaattttcttctctaaaatattcatttaaaatgcgttcgatagccgcatacgctgcatgttgtattaattctttattttgcac

SEQ ID NO: 16 - protein, 578 aa, Staphylococcus aureus
MQNKELIQHAAYAAIERILNEYFREENLYQVPPQNHQWSIQLSELETLTGEFRYWSAMGHHMYHPEVWLIDGKSKKITTYKEAIARILQHMAQSADNQTAVQQHMAQIMSDIDNSIHRTARYLQSNTIDYVEDRYIVSEQSLYLGHPFHPTPKSASGFSEADLEKYAPECHTSFQLHYLAVHQDVLLTRYVEGKEDQVEKVLYQLADIDISEIPKDFILLPTHPYQINVLRQHPQYMQYSEQGLIKDLGVSGDSVYPTSSVRTVFSKALNIYLKLPIHVKITNFIRTNDLEQIERTIDAAQVIASVKDEVETPHFKLMFEEGYRALLPNPLGQTVEPEMDLLTNSAMIVREGIPNYHADKDIHVLASLFETMPDSPMSKLSQVIEQSGLAPEAWLECYLNRTLLPILKLFSNTGISLEAHVQNTLIELKDGIPDVCFVRDLEGICLSRTIATEKQLVPNVVAASSPVVYAHDEAWHRLKYYVVVNHLGHLVSTIGKATRNEVVLWQLVAHRLMTWKKEYANNAVFVDCVEDLYQTPTIAAKANLMSKLNDCGANPIYTHIPNPICHNKEVSYCESNNS

SEQ ID NO: 17 - DNA, 1848 bp, Staphylococcus aureus
atgattgtggtgcaaaccctatttatacacatataccaaatccaatttgttataacaaggaggtatcgtattgtgaatcaaacaattcttaatcgtgtaaagactagagtaatgcatcaactggtatcatcacttatttatgagaatattgttgtgtataaagcgtcatatcaagacggtgtcggtcattttacaatagaaggacatgattcagagtatcgttttactgctgaaaagacacatagctttgatcgtatacgtatcacatcaccaattgagcgtgtcgtaggagatgaggcagatacaacaacagactatacacaattattgagagaggctgtatttacatttcctaaaaatgatgaaaagctagaacaatttattgttgagttattacagacagaattaaaagatacacaaagtatgcagtatcgagaatcaaacccaccagcaacacctgagacatttaacgactatgaattttatgcgatggaagggcatcagtatcatccaagttacaaatcacgtttaggatttacgttgagtgataatttgaaatttggtcctgattttgtaccaaacgttaaactgcagtggttagctatcgacaaagataaagtagaaacgacggtatcaagaaatgttgtagttaacgaaatgttacgtcaacaagttggcgataagacttatgaacattttgtacagcaaattgaagcgtctggcaaacatgtaaatgatgttgagatgatacctgtacacccatggcagtttgaacatgtcatccaagttgatttggctgaagaaaggcttaatggcacagtactatggttaggggaaagtgatgagctatatcaccctcaacaatcgattcgtacgatgtcgccaatagacacgacaaaatattatttaaaggtaccaataagtataacgaacacttcaacgaaacgagtgttggcgcctcatacaattgaaaatgcagcgcaaattacggattggttaaagcagatacagcaacaagatatgtatttaaaagatgaattaaagacagtttttctaggggaagtcttaggacagtcttatttaaatacacaactttcgccttataaacaaactcaagtttatggtgcgttaggtgttatatggcgtgaaaatatatatcatatgttaatcgatgaagaggatgcgataccatttaatgcactttatgcaagtgataaggatggtttaccattcattgaaaagtggattaaacaatatggttctgaagcttggacaaagcaatttttagctgtagcgattcgtccaatgattcatatgctttattatcacggtattgcctttgaatcgcatgcacaaaatatgatgctcattcatgaaaatggttggcctacacgtattgccttaaaagatttccacgatggtgttcgttttaagcgtgagcatttaagtgaagcagcttcacacctgacattaaagccaatgccagaagcacataaaaaagtgaatagtaattcatttattgaaacagatgacgaacgtttagtacgcgactttttacatgatgcatttttctttattaatatcgccgaaatcatcttatttattgaaaagcaatatggtatcgatgagcagcgacaatggcaatgggttaaagacattatcgaggcgtatcaagaagcatttccagagttgaataactatcaacatttcgatttgtttgaacctacgattcaagttgaaaagttaacgacacgtcgattattaagtgactccgagttaagaattcatcatgttacaaatccattaggtgtaggaggtatcaatgatgcaacaactatctctgaaaca

SEQ ID NO: 18 - DNA, 1848 bp, Staphylococcus aureus
tgtttcagagatagttgttgcatcattgatacctcctacacctaatggatttgtaacatgatgaattcttaactcggagtcacttaataatcgacgtgtcgttaacttttcaacttgaatcgtaggttcaaacaaatcgaaatgttgatagttattcaactctggaaatgcttcttgatacgcctcgataatgtctttaacccattgccattgtcgctgctcatcgataccatattgcttttcaataaataagatgatttcggcgatattaataaagaaaaatgcatcatgtaaaaagtcgcgtactaaacgttcgtcatctgtttcaataaatgaattactattcacttttttatgtgcttctggcattggctttaatgtcaggtgtgaagctgcttcacttaaatgctcacgcttaaaacgaacaccatcgtggaaatcttttaaggcaatacgtgtaggccaaccattttcatgaatgagcatcatattttgtgcatgcgattcaaaggcaataccgtgataataaagcatatgaatcattggacgaatcgctacagctaaaaattgctttgtccaagcttcagaaccatattgtttaatccacttttcaatgaatggtaaaccatccttatcacttgcataaagtgcattaaatggtatcgcatcctcttcatcgattaacatatgatatatattttcacgccatataacacctaacgcaccataaacttgagtttgtttataaggcgaaagttgtgtatttaaataagactgtcctaagacttcccctagaaaaactgtctttaattcatcttttaaatacatatcttgttgctgtatctgctttaaccaatccgtaatttgcgctgcattttcaattgtatgaggcgccaacactcgtttcgttgaagtgttcgttatacttattggtacctttaaataatattttgtcgtgtctattggcgacatcgtacgaatcgattgttgagggtgatatagctcatcactttcccctaaccatagtactgtgccattaagcctttcttcagccaaatcaacttggatgacatgttcaaactgccatgggtgtacaggtatcatctcaacatcatttacatgtttgccagacgcttcaatttgctgtacaaaatgttcataagtcttatcgccaacttgttgacgtaacatttcgttaactacaacatttcttgataccgtcgtttctactttatctttgtcgatagctaaccactgcagtttaacgtttggtacaaaatcaggaccaaatttcaaattatcactcaacgtaaatcctaaacgtgatttgtaacttggatgatactgatgcccttccatcgcataaaattcatagtcgttaaatgtctcaggtgttgctggtgggtttgattctcgatactgcatactttgtgtatcttttaattctgtctgtaataactcaacaataaattgttctagcttttcatcatttttaggaaatgtaaatacagcctctctcaataattgtgtatagtctgttgttgtatctgcctcatctcctacgacacgctcaattggtgatgtgatacgtatacgatcaaagctatgtgtcttttcagcagtaaaacgatactctgaatcatgtccttctattgtaaaatgaccgacaccgtcttgatatgacgctttatacacaacaatattctcataaataagtgatgataccagttgatgcattactctagtctttacacgattaagaattgtttgattcacaatacgatacctccttgttataacaaattggatttggtatatgtgtataaatagggtttgcaccacaatcat

SEQ ID NO: 19 - protein, 579 aa, Staphylococcus aureus
MHQLVSSLIYENIVVYKASYQDGVGHFTIEGHDSEYRFTAEKTHSFDRIRITSPIERVVGDEADTTTDYTQLLREVVFTFPKNDEKLEQFIVELLQTELKDTQSMQYRESNPPATPETFNDYEFYAMEGHQYHPSYKSRLGFTLSDNLKFGPDFVPNVKLQWLAIDKDKVETTVSRNVVVNEMLRQQVGDKTYEHFVQQIEASGKHVNDVEMIPVHPWQFEHVIQVDLAEERLNGTVLWLGESDELYHPQQSIRTMSPIDTTKYYLKVPISITNTSTKRVLAPHTIENAAQITDWLKQIQQQDMYLKDELKTVFLGEVLGQSYLNTQLSPYKQTQVYGALGVIWRENIYHMLIDEEDAIPFNALYASDKDGVPFIENWIKQYGSEAWTKQFLAVAIRPMIHMLYYHGIAFESHAQNMMLIHENGWPTRIALKDFHDGVRFKREHLSEAASHLTLKPMPEAHKKVNSNSFIETDDERLVRDFLHDAFFFINIAEIILFIEKQYGIDEELQWQWVKGIIEAYQEAFPELNNYQHFDLFEPTIQVEKLTTRRLLSDSELRIHHVTNPLGVGGINDATTISET

SEQ ID NO: 20 - DNA, 777 bp, Staphylococcus aureus
atgatgcaacaactatctctgaaacatagattaaacaatggtgattcagtttatggcatttttaattctataccggacccattgatgatcgaggttatcgcagcaagcgggtatgactttgttgtgattgatacagaacacgtggcgattaatgatgagacactagcgcatttaattcgtgcagctgaagcagcgcatattataccaattgtacgtgtcactgcagtgatagatagagatatcattaaagtgttagatatgggtgcgagaggtattattgtgccacacgttaaagatcgtgagacagttgagcatattgtgaaattaagtcgttattacccgcaaggattaagaagtttgaatggtggtcgcatggcaagatttggacgtacaccattacttgatgcaatggagatggctaatgagcatattatggtgattgccatgatagaagatgttgaaggggttatggccattgacgatatagctcaagtcgaaggtttagacatgatagtcgaaggtgccgcagatttatcgcagtcacttggcataccatggcaaacgcgtgatgatcaagtaacatcacatgttcaacatatttttgaggttgtgaatgcacatggtaaacatttttgtgcattaccacgtgaagatgaagatattgcaaaatggcaggcacaaggtgtacaaacatttattttaggtgatgatcgcggaaaaatatatcgccatttaagtgcatctctagcgacgtctaaacagaaaggggatgatggc

SEQ ID NO: 21 - DNA, 777 bp, Staphylococcus aureus
gccatcatcccctttctgtttagacgtcgctagagatgcacttaaatggcgatatatttttccgcgatcatcacctaaaataaatgtttgtacaccttgtgcctgccattttgcaatatcttcatcttcacgtggtaatgcacaaaaatgtttaccatgtgcattcacaacctcaaaaatatgttgaacatgtgatgttacttgatcatcacgcgtttgccatggtatgccaagtgactgcgataaatctgcggcaccttcgactatcatgtctaaaccttcgacttgagctatatcgtcaatggccataaccccttcaacatcttctatcatggcaatcaccataatatgctcattagccatctccattgcatcaagtaatggtgtacgtccaaatcttgccatgcgaccaccattcaaacttcttaatccttgcgggtaataacgacttaatttcacaatatgctcaactgtctcacgatctttaacgtgtggcacaataatacctctcgcacccatatctaacactttaatgatatctctatctatcactgcagtgacacgtacaattggtataatatgcgctgcttcagctgcacgaattaaatgcgctagtgtctcatcattaatcgccacgtgttctgtatcaatcacaacaaagtcatacccgcttgctgcgataacctcgatcatcaatgggtccggtatagaattaaaaatgccataaactgaatcaccattgtttaatctatgtttcagagatagttgttgcatcat

SEQ ID NO: 22 - protein, 259 aa, Staphylococcus aureus
MMQQLSLKHRLNNGDSVYGIFNSIPDPLMIEVIAASGYDFVVIDTEHVAINDETLAHLIRAAEAAHIIPIVRVTAVIDRDIIKVLDMGARGIIVPHVKDRETVEHIVKLSRYYPQGLRSLNGGRMARFGRTPLLDAMEMANEHIMVIAMIEDVEGVMAIDDIAQVEGLDMIVEGAADLSQSLGIPWQTRDDQVTSHVQHIFEVVNAHGKHFCALPREDEDIAKWQAQGVQTFILGDDRGKIYRHLSASLATSKQKGDEG

SEQ ID NO: 23 - DNA, 1200 bp, Staphylococcus aureus
atgcgtatagttcaacctgttattgaacaattaaaagcacaatctcatccagtttgtcattatatctatgatttagtcggactggaacatcatttgcaacatattacatcgtcattgccgagtaattgtcaaatgtactatgcaatgaaagcaaatagtgaacgaacaatcctagatacaattagtcagtatgttgaaggattcgaagttgcatctcaaggtgaaatagcaaaaggtcttgcttttaaaccagcaaatcatattatttttggtggccctggtaagacagacgaggaactaagatatgcagtaagtgaaggtgttcagcgtattcatgttgaaagtatgcatgaattacaacggctaaatgccatcttagaagatgaagataagacacaacacattttattgcgtgttaatttagcaggaccatttcccaatgcaacgttgcatatggcaggacgcccaacacaatttggtatttctgaagacgaagttgatgatgtcattgaagctgcgctagtaatgccaaatattcatctagatggctttcattttcattctatttctaacaatttagactcgaatttacatgtcgatgtagtgaaactttattttaaaaaagcaaaatcatggtctgaaaaacatcgatttccactcaaacatatcaatcttggtggtggcataggcgttaactatgcagatttaactagccaatttgagtgggataattttgtagaaaattttaaaacacttatcgttgagcaagaaatggaagatgtgacattgaactttgaatgtgggcgctttattgtggcacatattggttactatgtgacagaagtgctagatattaagaaagtgcatggtgcttggtatgccattttaagaggaggtacgcaacaatttagactgccggtatcttggcagcataaccatccttttgaaatttatcgttataaggacaatccatattcatttgaaaaagtttcaatttcgagacaggacacaacgttagtcggtcaattatgtacaccgaaagatgtctttgctagagaagtacagatagacgcaatcagtacaggcgacgttattgttttcaaatatgcaggtgcatacggatggtctatttcacatcacgatttcttaagccatccacatcctgaatttatttatttaacgcaaacaaaggaggatgaa

SEQ ID NO: 24 - DNA, 1200 bp, Staphylococcus aureus
ttcatcctcctttgtttgcgttaaataaataaattcaggatgtggatggcttaagaaatcgtgatgtgaaatagaccatccgtatgcacctgcatatttgaaaacaataacgtcgcctgtactgattgcgtctatctgtacttctctagcaaagacatctttcggtgtacataattgaccgactaacgttgtgtcctgtctcgaaattgaaactttttcaaatgaatatggattgtccttataacgataaatttcaaaaggatggttatgctgccaagataccggcagtctaaattgttgcgtacctcctcttaaaatggcataccaagcaccatgcactttcttaatatctagcacttctgtcacatagtaaccaatatgtgccacaataaagcgcccacattcaaagttcaatgtcacatcttccatttcttgctcaacgataagtgttttaaaattttctacaaaattatcccactcaaattggctagttaaatctgcatagttaacgcctatgccaccaccaagattgatatgtttgagtggaaatcgatgtttttcagaccatgattttgcttttttaaaataaagtttcactacatcgacatgtaaattcgagtctaaattgttagaaatagaatgaaaatgaaagccatctagatgaatatttggcattactagcgcagcttcaatgacatcatcaacttcgtcttcagaaataccaaattgtgttgggcgtcctgccatatgcaacgttgcattgggaaatggtcctgctaaattaacacgcaataaaatgtgttgtgtcttatcttcatcttctaagatggcatttagccgttgtaattcatgcatactttcaacatgaatacgctgaacaccttcacttactgcatatcttagttcctcgtctgtcttaccagggccaccaaaaataatatgatttgctggtttaaaagcaagaccttttgctatttcaccttgagatgcaacttcgaatccttcaacatactgactaattgtatctaggattgttcgttcactatttgctttcattgcatagtacatttgacaattactcggcaatgacgatgtaatatgttgcaaatgatgttccagtccgactaaatcatagatataatgacaaactggatgagattgtgcttttaattgttcaataacaggttgaactatacgcat

SEQ ID NO: 25 - protein, 400 aa, Staphylococcus aureus
MRIVQPVIEQLKAQSHPVCHYIYDLVGLEHHLQHITSSLPSNCQMYYAMKANSERKILDTISQYVEGFEVASQGEIAKGLAFKPANHIIFGGPGKTDEELRYAVSEGVQRIHVESMHELQRLNAILEDEDKTQHILLRVNLAGPFPNATLHMAGRPTQFGISEDEVDDVIEAALAMPKIHLDGFHFHSISNNLDSNLHVDVVKLYFKKAKAWSEKHRFPLKHINLGGGIGVNYADLTNQFEWDNFVERFKTLIVEQEMEDVTLNFECGRFIVAHIGYYVTEVLDIKKVHGAWYAILRGGTQQFRLPVSWQHNHPFDIYRYKDNPYSFEKVSISRQDTTLVGQLCTPKDVFAREVQIDAISTGDVIVFKYAGAYGWSISHHDFLSHPHPEFIYLTQTKEDE

SEQ ID NO: 26 - DNA, 762 bp, Staphylococcus aureus
ttgaatcatattcatgaacatttaaaattggtaccagtagataagattgatcttcacgaaacattcgaacctttaagattggaaaaaacgaaaagtagtattgaagcagatgattttatacgtcatcctattttagtgacagcgatgcaacatggtagatatatggttatagatggtgtgcatcggtatacaagtttgaaagcgttaggatgtaagaaagttccagtgcaagaaatccatgaaacacaatattcaattagtacatggcaacataaagttccatttggtgtgtggtgggaaacgttacaacaagaacatcgcttgccatggactactgagacaagacaagaagcgccatttattacgatgtgtcatggtgatacagaacaatatttgtatacgaaagatttaggcgaagcacattttcaagtatgggaaaaggttgtcgcaagttatagtggttgttgttctgtagagagaattgcacaaggtacatatccttgtctttctcaacaagatgtactcatgaagtatcagccattgagttataaggaaattgaagcggttgttcataaaggggaaactgtgccagcaggtgtgacacgctttaatatttcaggacgatgtcttaatcttcaagtaccactggcattacttaaacaagatgatgatgttgaacaactgcgtaattggaagcagtttttagcagataagtttgccaatatgagatgctatactgaaaaagtatacttggtggagcaa

SEQ ID NO: 27 - DNA, 762 bp, Staphylococcus aureus
ttgctccaccaagtatactttttcagtatagcatctcatattggcaaacttatctgctaaaaactgcttccaattacgcagttgttcaacatcatcatcttgtttaagtaatgccagtggtacttgaagattaagacatcgtcctgaaatattaaagcgtgtcacacctgctggcacagtttcccctttatgaacaaccgcttcaatttccttataactcaatggctgatacttcatgagtacatcttgttgagaaagacaaggatatgtaccttgtgcaattctctctacagaacaacaaccactataacttgcgacaaccttttcccatacttgaaaatgtgcttcgcctaaatctttcgtatacaaatattgttctgtatcaccatgacacatcgtaataaatggcgcttcttgtcttgtctcagtagtccatggcaagcgatgttcttgttgtaacgtttcccaccacacaccaaatggaactttatgttgccatgtactaattgaatattgtgtttcatggatttcttgcactggaactttcttacatcctaacgctttcaaacttgtataccgatgcacaccatctataaccatatatctaccatgttgcatcgctgtcactaaaataggatgacgtataaaatcatctgcttcaatactacttttcgttttttccaatcttaaaggttcgaatgtttcgtgaagatcaatcttatctactggtaccaattttaaatgttcatgaatatgattcaa

SEQ ID NO: 28 - protein, 254 aa, Staphylococcus aureus
MNHIHEHLKLVPVDKIDLHETFEPLRLEKTKSSIEADDFIRHPILVTAMQHGRYMVIDGVHRYTSLKALGCKKVPVQEIHETQYSISTWQHKVPFGVWWETLQQEHRLPWTTETRQEAPFITMCHGDTEQYLYTKDLGEAHFQVWEKVVASYSGCCSVERIAQGTYPCLSQQDVLMKYQPLSYKEIEAVVHKGETVPAGVTRFNISGRCLNLQVPLALLKQDDDVEQLRNWKQFLADKFANMRCYTEKVYLVEQ

SEQ ID NO: 29 - DNA, 236 bp, Staphylococcus aureus
cattgactaattagcctccttcgtgatgtatgacaatgagaatcattatcacgatttagtatgaattaaattttttcctaagtcaataaaatatttatgatttacatgcaacttataattatttgacatataaatgcataaaaaatataatcctaattacttgatagtgagaatcattatcaattaggtaacacacaatattatagaattttaaatttgaggaggaagcgcttttg

SEQ ID NO: 30 - DNA, 236 bp, Staphylococcus aureus
caaaagcgcttcctcctcaaatttaaaattctataatattgtgtgttacctaattgataatgattctcactatcaagtaattaggattatattttttatgcatttatatgtcaaataattataagttgcatgtaaatcataaatattttattgacttaggaaaaaatttaattcatactaaatcgtgataatgattctcattgtcatacatcacgaaggaggctaattagtcaatg

SEQ ID NO: 31 - DNA, 19 bp, Staphylococcus aureus
tgagaatcattatcaatta

SEQ ID NO: 32 - DNA, 28 bp, Artificial Sequence
  Description of Artificial Sequence Synthetic oligonucleotide
ttggatccagtatatgaatcctggaggc

SEQ ID NO: 33 - DNA, 32 bp, Artificial Sequence
  Description of Artificial Sequence Synthetic oligonucleotide
ttggatccaaaaatgactgaccctttcgcatc

SEQ ID NO: 34 - DNA, 36 bp, Artificial Sequence
  Description of Artificial Sequence Synthetic oligonucleotide
tggatcccatcaccaattgagcgtgtcgtaggagat

SEQ ID NO: 35 - DNA, 36 bp, Artificial Sequence
  Description of Artificial Sequence Synthetic oligonucleotide
tggatcctttcaattgtatgaggcgccaacactcgt

SEQ ID NO: 36 - DNA, 30 bp, Artificial Sequence
  Description of Artificial Sequence Synthetic oligonucleotide
ttgcggccgcgatagatagagatatcatta

SEQ ID NO: 37 - DNA, 28 bp, Artificial Sequence
  Description of Artificial Sequence Synthetic oligonucleotide
ttggatcctagttaacgcctatgccacc

SEQ ID NO: 38 - DNA, 35 bp, Artificial Sequence
  Description of Artificial Sequence Synthetic oligonucleotide
ttgcggccgccccaacacaatttggtatttctgaa

SEQ ID NO: 39 - DNA, 28 bp, Artificial Sequence
  Description of Artificial Sequence Synthetic oligonucleotide
ttggatcctacttgaaaatgtgcttcgc

SEQ ID NO: 40 - DNA, 30 bp, Artificial Sequence
  Description of Artificial Sequence Synthetic oligonucleotide
ttgcggccgcaagttccatttggtgtgtgg

SEQ ID NO: 41 - DNA, 28 bp, Artificial Sequence
  Description of Artificial Sequence Synthetic oligonucleotide
ttggatccggtaaaacagtgaaaagagc

SEQ ID NO: 42 - DNA, 30 bp, Artificial Sequence
  Description of Artificial Sequence Synthetic oligonucleotide
ttgcggccgctattatcgctttagtattat

SEQ ID NO: 43 - DNA, 29 bp, Artificial Sequence
  Description of Artificial Sequence Synthetic oligonucleotide
ttggatcctcaacgcctgcttgagatgtt

SEQ ID NO: 44 - DNA, 28 bp, Artificial Sequence
  Description of Artificial Sequence Synthetic oligonucleotide
ttggatccattagcagacatagatatat

SEQ ID NO: 45 - DNA, 28 bp, Artificial Sequence
  Description of Artificial Sequence Synthetic oligonucleotide
ttggatcctagtgtctcatcattaatcg

(19) United States
(12) Patent Application Publication
(10) Pub. No.: US 2011/0206685 A1
(43) Pub. Date: Aug. 25, 2011

(54) SCREENING ASSAYS FOR INHIBITORS OF A STAPHYLOCOCCUS AUREUS SIDEROPHORE

(76) Inventors: David E. Heinrichs, London (CA); Suzanne Dale, Rochester, NY (US)

(21) Appl. No.: 11/574,959

(22) PCT Filed: Sep. 8, 2005
(86) PCT No.: PCT/IB2005/004081

§ 371 Date: Sep. 17, 2008

Related U.S. Application Data
(60) Provisional application No. 60/607,896, filed on Sep. 8, 2004.

(51) Int. Cl. A61K 39/40; C12N 1/20; A61K 31/708; C07K 14/31; C12N 9/10; C12N 9/88; A61P 31/04; A01N 37/18; A01N 43/90; A01P 1/00

(52) U.S. Cl. 424/158.1; 435/252.1; 514/44 A; 424/164.1; 530/350; 435/193; 435/232

(57) ABSTRACT

Isolation of an iron regulated, nme-gene operon (designated sbn) from Staphylococcus aureus (RN6390), responsible for the biosynthesis of staphylobactm, a novel S. aureus siderophore Methods for treating or preventing a disease or condition caused by S. aureus infection, as well as methods for identifying agents that inhibit the biosynthesis of staphylobactm or inhibit the expression of genes in said sbn operon are further disclosed.

### CROSS-REFERENCE TO RELATED APPLICATIONS

This application claims priority to U.S. Provisional Application No. 60/607,896, which was filed on Sep. 8, 2004, the contents of which are hereby incorporated by reference in their entirety.

### BACKGROUND

Iron is an absolute requirement for the growth of most microorganisms, with the possible exceptions of lactobacilli (Archibald (1983) FEMS Microbiol. Lett. 19:29-32) and Borrelia burgdorferi (Posey and Gherardini (2000) Science 288:1651-1653). Despite being the fourth most abundant element on the Earth's crust, iron is frequently a growth-limiting nutrient. In aerobic environments and at physiological pH, iron is present in the ferric (Fe³⁺) state and forms insoluble hydroxide and oxyhydroxide precipitates. Mammals overcome iron restriction by possessing high-affinity iron-binding glycoproteins such as transferrin and lactoferrin that serve to solubilize and deliver iron to host cells (Weinberg (1999) Emerg. Infect. Dis. 5:346-352). This results in a further restriction of free extracellular iron and, accordingly, the concentration of free iron in the human body is estimated to be 10⁻¹⁸ M, a concentration that is several, orders lower than that required to support a productive bacterial infection (Braun et al., (1998) Bacterial iron transport: mechanisms, genetics, and regulation, p. 67-145. In A. Sigel and H. Sigel (ed.), Metal Ions in Biological Systems, vol. 35. Iron transport and storage in microorganisms, plants, and animals. Marcel Dekker, Inc., New York).

To overcome iron restriction, bacteria have evolved several different mechanisms to acquire this essential nutrient. For example, members of the Pasteurellaceae may express receptors for the recognition of iron-loaded forms of transferrin and lactoferrin (Gray-Owen and Schryvers, (1996) Trends Microbiol. 4:185-91). One of the most common iron acquisition mechanisms, though, is through the use of low-molecular-weight, high-affinity iron chelators, termed siderophores, and cognate cell envelope receptors that serve to actively internalize ferric-siderophore complexes. Many siderophores are able to successfully compete with transferrin and lactoferrin for host iron. Indeed, the expression of ferric-siderophore uptake systems are critical virulence factors in bacteria such as septicemic E. coli (Williams (1979) Infect. Immun. 26:925-932), Vibrio anguillarum (Crosa et al. (1980) Infect. Immun. 27:897-902), Erwinia chrysanthemi (Enard et al., (1988) J. Bacteriol. 170:2419-2426) and Pseudomonas aeruginosa (Meyer et al. (1996) Infect. Immun. 64:518-523).

Staphylococcus aureus (S. aureus) possesses several different iron-regulated ABC transporters, including those encoded by the sstABCD (Morrissey et al. (2000) Infect. Immun. 68:6281-6288), sirABC (Heinrichs et al. (1999) J. Bacteriol. 181:1436-1443) and fhuCBG (Sebulsky et al. (2000) J. Bacteriol. 182:4394-4400) operons. While the transported substrates are unknown for the sst and sir systems, the fhuCBG genes, in concert with fhuD1 and fhuD2 (Sebulsky and Heinrichs (2001) J. Bacteriol. 183:4994-5000), are involved in the acquisition of iron(III)-hydroxamate complexes. Several members of the staphylococci, including numerous coagulase-negative staphylococci (CoNS) and strains of S. aureus, produce siderophores. Two of these siderophores, staphyloferrin A (Konetscny-Rapp et al., (1990) Eur. J. Biochem. 191:65-74; Meiwes et al. (1990) FEMS Microbiol. Lett. 67:201-206) and staphyloferrin B (Dreschel et al. (1993) BioMetals. 6:185-192; Haag et al. (1994) FEMS Microbiol. Lett. 115:125-130), are of the polycarboxylate class, while the third, aureochelin (Courcol et al. (1997) Infect. Immun. 65:1944-1948), is chemically uncharacterized. Leading into our study, no molecular-genetic information was known about the synthesis of any of the staphylococcal siderophores.

S. aureus is a prevalent human pathogen that causes a wide range of infections ranging from minor skin and wound infections to more serious sequelae such as endocarditis, osteomyelitis and septicemia (Archer (1998) Clin. Infect. Dis. 26:1179-1181). The ability of S. aureus to invade and colonize many tissues may be ascribed to its capacity to express several virulence factors such as fibronectin-, elastin- and collagen-binding proteins that aid in tissue adherence, and multiple exotoxins and proteases that result in tissue destruction and bacterial dissemination. The ability of this bacterium to acquire iron during in vivo growth is also likely important to its pathogenesis, and several research groups have characterized several different genes whose products are involved in the binding and/or transport of host iron compounds (Mazmanian et al. (2003) Science 299:906-9; Modun et al. (1998) Infect. Immun. 66:3591-3596; Taylor and Heinrichs (2002) Mol. Microbiol. 43:1603-1614).

Initially, penicillin could be used to treat even the worst S. aureus infections. However, the emergence of penicillin-resistant strains of S. aureus has reduced the effectiveness of penicillin in treating S. aureus infections and most strains of S. aureus encountered in hospital infections today do not respond to penicillin. Penicillin-resistant strains of S. aureus produce a lactamase which converts penicillin to pencillinoic acid, and thereby destroys antibiotic activity. Furthermore, the lactamase gene often is propagated episomally, typically on a plasmid, and often is only one of several genes on an episomal element that, together, confer multidrug resistance.

Methicillins, introduced in the 1960s, largely overcame the problem of penicillin resistance in S. aureus. These compounds conserve the portions of penicillin responsible for antibiotic activity and modify or alter other portions that make penicillin a good substrate for inactivating lactamases. However, methicillin resistance has emerged in S. aureus, along with resistance to many other antibiotics effective against this organism, including aminoglycosides, tetracycline, chloramphenicol, macrolides and lincosamides. In fact, methicillin-resistant strains of S. aureus generally are multiply drug resistant. Methicillian-resistant S. aureus (MRSA) has become one of the most important nosocomial pathogens worldwide and poses serious infection control problems. Today, many strains are multiresistant against virtually all antibiotics with the exception of vancomycin-type glycopeptide antibiotics. Drug resistance of S. aureus infections poses significant treatment difficulties, which are likely to get much worse unless new therapeutic agents are developed.

There is thus an urgent unmet medical need for new and effective therapeutic agents to treat S. aureus infections.

### SUMMARY OF THE INVENTION

The present invention is based, at least in part, on the identification and characterization of an iron-regulated, nine gene operon (designated sbn) whose products are involved in the biosynthesis of a siderophore in S. aureus. Expression of the sbn operon is not only important for iron-restricted growth of S. aureus in laboratory culture, but also is important for S. aureus to survive in vivo. As a result, the genes and proteins involved with this siderophore's biosynthesis are important drug targets that can be used in screening assays to identify S. aureus specific antibiotics.

In one aspect, the invention features each of the nine genes comprising the sbn operon (i.e., sbnA, sbnB, sbnC, sbnD, sbnE, sbnF, sbnG, sbnH, and sbnI), recombinant vectors containing sbn genes, host cells containing the recombinant vectors and methods of producing the encoded polypeptides.

In another aspect, the invention features Sbn polypeptides encoded by each of the genes of the sbn operon. The Sbn polypeptides comprise SbnA, SbnB, SbnC, SbnD, SbnE, SbnF, SbnG, SbnH, and SbnI. Each Sbn polypeptide is required for the biosynthesis of the S. aureus siderophore (which is also referred to as “staphylobactin”).

In another aspect, the invention features novel antibiotics, including antibodies, antisense RNAs, and siRNAs that inhibit iron uptake in Staphylococcus aureus (S. aureus).

A further aspect of the invention features screening assays for identifying agents that inhibit staphylobactin biosynthesis in S. aureus. In one embodiment, the assay can identify agents that bind to a sbn gene product and thereby interfere with its biochemical function. In another embodiment, the assay can identify agents that inhibit the expression of Sbn polypeptides and/or nucleic acids in S. aureus.

Further features and advantages of the instant disclosed inventions will now be discussed in conjunction with the following Detailed Description and Claims.

### BRIEF DESCRIPTION OF THE DRAWINGS

FIG. 1 shows the nucleic acid sequence of the sbn operon (SEQ ID NO: 1).

FIG. 2 shows (A) the nucleic acid sequence (SEQ ID NO: 2), (B) the reverse complement of SEQ ID NO: 2 (SEQ ID NO: 3), and (C) the amino acid sequence of SbnA (SEQ ID NO: 4).

FIG. 3 shows (A) the nucleic acid sequence (SEQ ID NO: 5), (B) the reverse complement of SEQ ID NO: 5 (SEQ ID NO: 6), and (C) the amino acid sequence of SbnB (SEQ ID NO: 7).

FIG. 4 shows (A) the nucleic acid sequence (SEQ ID NO: 8), (B) the reverse complement of SEQ ID NO: 8 (SEQ ID NO: 9), and (C) the amino acid sequence of SbnC (SEQ ID NO: 10).

FIG. 5 shows (A) the nucleic acid sequence (SEQ ID NO: 11), (B) the reverse complement of SEQ ID NO: 11 (SEQ ID NO: 12), and (C) the amino acid sequence of SbnD (SEQ ID NO: 13).

FIG. 6 shows (A) the nucleic acid sequence (SEQ ID NO: 14), (B) the reverse complement of SEQ ID NO: 14 (SEQ ID NO: 15), and (C) the amino acid sequence of SbnE (SEQ ID NO: 16).

FIG. 7 shows (A) the nucleic acid sequence (SEQ ID NO: 17), (B) the reverse complement of SEQ ID NO: 17 (SEQ ID NO: 18), and (C) the amino acid sequence of SbnF (SEQ ID NO: 19).

FIG. 8 shows (A) the nucleic acid sequence (SEQ ID NO: 20), (B) the reverse complement of SEQ ID NO: 20 (SEQ ID NO: 21), and (C) the amino acid sequence of SbnG (SEQ ID NO: 22).

FIG. 9 shows (A) the nucleic acid sequence (SEQ ID NO: 23), (B) the reverse complement of SEQ ID NO: 23 (SEQ ID NO: 24), and (C) the amino acid sequence of SbnH (SEQ ID NO: 25).

FIG. 10 shows (A) the nucleic acid sequence (SEQ ID NO: 26), (B) the reverse complement of SEQ ID NO: 26 (SEQ ID NO: 27), and (C) the amino acid sequence of SbnI (SEQ ID NO: 28).

FIG. 11 shows siderophore levels in spent culture supernatants of RN6390, Newman, and their respective fur derivatives, H295 and H706. Bacteria were grown in an iron-deficient (open bars) or an iron-replete (iron-deficient medium supplemented with 50 μM iron chloride) (gray bars) medium, while the fur::km derivatives of both RN6390 and Newman (solid bars) were grown in an iron-replete medium. Siderophore units were calculated as described in Example 1.

FIG. 12 shows a schematic representation of the sir-galE region of the S. aureus chromosome. Arrows are representative of individual coding regions. The coding regions within the sbn operon are represented by open arrows, the sir coding regions are shown with gray arrows, and coding regions likely not involved in iron uptake are shown in black arrows. SA0121 is a hypothetical open reading frame (orf) with nomenclature that is derived from the N315 genome sequence. Bud is a putative butanediol dehydrogenase and galE encodes a UDP-galactose-4-epimerase.

FIG. 13 shows the promoter region for the sirABC and sbn operons (sense strand, SEQ ID NO: 29; antisense strand, SEQ ID NO: 30). Putative Fur box sequences are boxed. Also shown are the predicted start codons for the sirA and sbnA genes, along with predicted Shine-Dalgarno (S.D.) sequences.

FIGS. 14A-B are graphs showing the effect of a sbnE mutation on the growth of S. aureus. Growth curve of S. aureus RN6390 (∘), Newman (◯), H672 (RN6390 sbnE::Km) (▾), H686 (Newman sbnE::Km) (∇), H672+pSED32 (▪) and H686+pSED32 (□) grown in TMS medium supplemented with 10 μM EDDHA in the presence (Panel A) or absence (Panel B) of 50 μM FeCl₃. Bacteria were grown in side-arm flasks with vigorous shaking, and growth was monitored using a Klett meter. Growth experiments were performed in duplicate in three separate experiments. The results of a typical experiment are shown.

FIG. 15 is a graph showing that a sbnE mutant is compromised in a murine kidney abscess model. Two groups of twelve mice were injected in the tail vein with 1×10⁷ bacteria. One group received S. aureus Newman, while the second group was infected with H686 (Newman sbnE::Km). CFU recovered from the kidneys of mice at both five (8 mice) and six (4 mice) day post-infection are plotted. Each symbol represents the staphylococcal count in the kidneys of one animal and the dashed line represents the limit of detection for staphylococci in this assay system. Data are representative of three independent experiments. Statistical significance was determined using the Student unpaired t test and found to be highly significant (P<0.003).

### DETAILED DESCRIPTION

### 1. General

The present invention is based, at least in part, on the discovery of the role of the Staphylococcus aureus (S. aureus) sbn operon in the biosynthesis of a siderophore, which is referred to as staphylobactin. Siderophores are high-affinity iron chelators that bacteria use to acquire iron required for bacterial growth. Described herein are novel antibiotics that inhibit siderophore production in S. aureus and method for screening compounds to identify additional inhibitors of siderophore biosynthesis.

### 2. Definitions

For convenience, the meaning of certain terms and phrases employed in the specification, examples, and appended claims are provided below. Unless defined otherwise, all technical and scientific terms used herein have the same meaning as commonly understood by one of ordinary skill in the art to which this invention belongs.

The term “agent” is used herein to denote a chemical compound, a mixture of chemical compounds, a biological macromolecule (such as a nucleic acid, an antibody, a protein or portion thereof, e.g., a peptide), or an extract made from biological materials such as bacteria, plants, fungi, or animal (particularly mammalian) cells or tissues. Agents may be identified by screening assays described herein below. Such agents may be inhibitors or antagonists of sbn mediated siderophore biosynthesis in Staphylococcus aureus. The activity of such agents may render it suitable as a “therapeutic agent” which is a biologically, physiologically, or pharmacologically active substance (or substances) that acts locally or systemically in a subject.

The terms “antagonist” or “inhibitor” refer to an agent that reduces or inhibits at least one bioactivity of a protein. An antagonist may be a compound which reduces or inhibits the interaction between a protein and another molecule, e.g., a target peptide or enzyme substrate. An antagonist may also be a compound that reduces or inhibits expression of a gene or which reduces or inhibits the amount of expressed protein present.

As used herein the term “antibody” refers to an immunoglobulin and any antigen-binding portion of an immunoglobulin (e.g., IgG, IgD, IgA, IgM and IgE) i.e., a polypeptide that contains an antigen binding site, which specifically binds (“immunoreacts with”) an antigen. Antibodies can comprise at least one heavy (H) chain and at least one light (L) chain interconnected by at least one disulfide bond. The term “V_{H}” refers to a heavy chain variable region of an antibody. The term “V_{L}” refers to a light chain variable region of an antibody. In exemplary embodiments, the term “antibody” specifically covers monoclonal and polyclonal antibodies. A “polyclonal antibody” refers to an antibody which has been derived from the sera of animals immunized with an antigen or antigens. A “monoclonal antibody” refers to an antibody produced by a single clone of hybridoma cells. Techniques for generating monoclonal antibodies include, but are not limited to, the hybridoma technique (see Kohler & Milstein (1975) Nature 256:495-497); the trioma technique; the human β-cell hybridoma technique (see Kozbor, et al. (1983) Immunol. Today 4:72), the EBV hybridoma technique (see Cole, et al., 1985 In: Monoclonal Antibodies and Cancer Therapy, Alan R. Liss, Inc., pp. 77-96) and phage display.

Polyclonal or monoclonal antibodies can be further manipulated or modified to generate chimeric or humanized antibodies. “Chimeric antibodies” are encoded by immunoglobulin genes that have been genetically engineered so that the light and heavy chain genes are composed of immunoglobulin gene segments belonging to different species. For example, substantial portions of the variable (V) segments of the genes from a mouse monoclonal antibody, e.g., obtained as described herein, may be joined to substantial portions of human constant (C) segments. Such a chimeric antibody is likely to be less antigenic to a human than a mouse monoclonal antibody.

As used herein, the term “humanized antibody” (HuAb) refers to a chimeric antibody with a framework region substantially identical (i.e., at least 85%) to a human framework, having CDRs from a non-human antibody, and in which any constant region has at least about 85-90%, and preferably about 95% polypeptide sequence identity to a human immunoglobulin constant region. See, for example, PCT Publication WO 90/07861 and European Patent No. 0451216. All parts of such a HuAb, except possibly the CDRs, are substantially identical to corresponding parts of one or more native human immunoglobulin sequences. The term “framework region” as used herein, refers to those portions of immunoglobulin light and heavy chain variable regions that are relatively conserved (i.e., other than the CDRs) among different immunoglobulins in a single species, as defined by Kabat, et al. (1987) Sequences of Proteins of Immunologic Interest, 4^{th} Ed., US Dept. Health and Human Services. Human constant region DNA sequences can be isolated in accordance with well known procedures from a variety of human cells, but preferably from immortalized B cells. The variable regions or CDRs for producing humanized antibodies may be derived from monoclonal antibodies capable of binding to the antigen, and will be produced in any convenient mammalian source, including mice, rats, rabbits, or other vertebrates.

The term “antibody” also encompasses antibody fragments. Examples of antibody fragments include Fab, Fab′, Fab′-SH, F(ab′)₂, and Fv fragments; diabodies and any antibody fragment that has a primary structure consisting of one uninterrupted sequence of contiguous amino acid residues, including without limitation: single-chain Fv (scFv) molecules, single chain polypeptides containing only one light chain variable domain, or a fragment thereof that contains the three CDRs of the light chain variable domain, without an associated heavy chain moiety and (3) single chain polypeptides containing only one heavy chain variable region, or a fragment thereof containing the three CDRs of the heavy chain variable region, without an associated light chain moiety; and multispecific or multivalent structures formed from antibody fragments. In an antibody fragment comprising one or more heavy chains, the heavy chain(s) can contain any constant domain sequence (e.g., CH1 in the IgG isotype) found in a non-Fc region of an intact antibody, and/or can contain any hinge region sequence found in an intact antibody, and/or can contain a leucine zipper sequence fused to or situated in the hinge region sequence or the constant domain sequence of the heavy chain(s). Suitable leucine zipper sequences include the jun and fos leucine zippers taught by Kostelney et al., (1992) J. Immunol., 148: 1547-1553 and the GCN4 leucine zipper described in U.S. Pat. No. 6,468,532. Fab and F(ab′)₂ fragments lack the Fc fragment of intact antibody and are typically produced by proteolytic cleavage, using enzymes such as papain (to produce Fab fragments) or pepsin (to produce F(ab′)₂ fragments).

An antibody “specifically binds” to an antigen or an epitope of an antigen if the antibody binds preferably to the antigen over most other antigens. For example, the antibody may have less than about 50%, 20%, 10%, 5%, 1% or 0.1% cross-reactivity toward one or more other epitopes.

The term “conservative substitutions” refers to changes between amino acids of broadly similar molecular properties. For example, interchanges within the aliphatic group alanine, valine, leucine and isoleucine can be considered as conservative. Sometimes substitution of glycine for one of these can also be considered conservative. Other conservative interchanges include those within the aliphatic group aspartate and glutamate; within the amide group asparagine and glutamine; within the hydroxyl group serine and threonine; within the aromatic group phenylalanine, tyrosine and tryptophan; within the basic group lysine, arginine and histidine; and within the sulfur-containing group methionine and cysteine. Sometimes substitution within the group methionine and leucine can also be considered conservative. Preferred conservative substitution groups are aspartate-glutamate; asparagine-glutamine; valine-leucine-isoleucine; alanine-valine; phenylalanine-tyrosine; and lysine-arginine.

An “effective amount” is an amount sufficient to produce a beneficial or desired clinical result upon treatment. An effective amount can be administered to a patient in one or more doses. In terms of treatment, an effective amount is an amount that is sufficient to decrease an infection in a patient. Several factors are typically taken into account when determining an appropriate dosage to achieve an effective amount. These factors include age, sex and weight of the patient, the condition being treated, the severity of the condition and the form and effective concentration of the agent administered.

“Equivalent” when used to describe nucleic acids or nucleotide sequences refers to nucleotide sequences encoding functionally equivalent polypeptides. Equivalent nucleotide sequences will include sequences that differ by one or more nucleotide substitution, addition or deletion, such as an allelic variant; and will, therefore, include sequences that differ due to the degeneracy of the genetic code. For example, nucleic acid variants may include those produced by nucleotide substitutions, deletions, or additions. The substitutions, deletions, or additions may involve one or more nucleotides. The variants may be altered in coding regions, non-coding regions, or both. Alterations in the coding regions may produce conservative or non-conservative amino acid substitutions, deletions or additions.

“Homology” or alternatively “identity” refers to sequence similarity between two peptides or between two nucleic acid molecules. Homology may be determined by comparing a position in each sequence which may be aligned for purposes of comparison. When a position in the compared sequence is occupied by the same base or amino acid, then the molecules are homologous at that position. A degree of homology between sequences is a function of the number of matching or homologous positions shared by the sequences. The term “percent identical” refers to sequence identity between two amino acid sequences or between two nucleotide sequences. Identity may be determined by comparing a position in each sequence which may be aligned for purposes of comparison. When an equivalent position in the compared sequences is occupied by the same base or amino acid, then the molecules are identical at that position; when the equivalent site is occupied by the same or a similar amino acid residue (e.g., similar in steric and/or electronic nature), then the molecules may be referred to as homologous (similar) at that position. Expression as a percentage of homology, similarity, or identity refers to a function of the number of identical or similar amino acids at positions shared by the compared sequences. Various alignment algorithms and/or programs may be used, including FASTA, BLAST, or ENTREZ. FASTA and BLAST are available as a part of the GCG sequence analysis package (University of Wisconsin, Madison, Wis.), and may be used with, e.g., default settings. ENTREZ is available through the National Center for Biotechnology Information, National Library of Medicine, National Institutes of Health, Bethesda, Md. In one embodiment, the percent identity of two sequences may be determined by the GCG program with a gap weight of 1, e.g., each amino acid gap is weighted as if it were a single amino acid or nucleotide mismatch between the two sequences. Other techniques for alignment are described in Methods in Enzymology, vol. 266: Computer Methods for Macromolecular Sequence Analysis (1996), ed. Doolittle, Academic Press, Inc., a division of Harcourt Brace & Co., San Diego, Calif., USA. Preferably, an alignment program that permits gaps in the sequence is utilized to align the sequences. The Smith-Waterman is one type of algorithm that permits gaps in sequence alignments. See Meth. Mol. Biol. 70: 173-187 (1997). Also, the GAP program using the Needleman and Wunsch alignment method may be utilized to align sequences. An alternative search strategy uses MPSRCH software, which runs on a MASPAR computer. MPSRCH uses a Smith-Waterman algorithm to score sequences on a massively parallel computer. This approach improves the ability to pick up distantly related matches, and is especially tolerant of small gaps and nucleotide sequence errors. Nucleic acid-encoded amino acid sequences may be used to search both protein and DNA databases. Databases with individual sequences are described in Methods in Enzymology, ed. Doolittle, supra. Databases include Genbank, EMBL, and DNA Database of Japan, (DDBJ).

As used herein, the term “infection” refers to an invasion and the multiplication of microorganisms such as S. aureus in body tissues, which may be clinically unapparent or result in local cellular injury due to competitive metabolism, toxins, intracellular replication or antigen antibody response. The infection may remain localized, subclinical and temporary if the body's defensive mechanisms are effective. A local infection may persist and spread by extension to become an acute, subacute or chronic clinical infection or disease state. A local infection may also become systemic when the microorganisms gain access to the lymphatic or vascular system. An infection of S. aureus may result in a disease or condition, including but not limited to a furuncle, chronic furunculosis, impetigo, acute osteomyelitis, pneumonia, endocarditis, scalded skin syndrome, toxic shock syndrome, and food poisoning.

The term “inhibit” refers to any decrease, reduction or complete inhibition of biological activity, nucleic acid expression, or protein expression.

“Label” and “detectable label” refer to a molecule capable of detection including, but not limited to radioactive isotopes, fluorophores, chemiluminescent moieties, enzymes, enzyme substrates, enzyme cofactors, enzyme inhibitors, dyes, metal ions, ligands (e.g., biotin or haptens) and the like. “Fluorophore” refers to a substance or a portion thereof which is capable of exhibiting fluorescence in the detectable range. Particular examples of appropriate labels include fluorescein, rhodamine; dansyl, umbelliferone, Texas red, luminol, NADPH, alpha- or beta-galactosidase and horseradish peroxidase.

As used herein with respect to genes, the term “mutant” refers to a gene which encodes a mutant protein. As used herein with respect to proteins, the term “mutant” means a protein which does not perform its usual or normal physiological role. S. aureus polypeptide mutants may be produced by amino acid substitutions, deletions or additions. The substitutions, deletions, or additions may involve one or more residues. Especially preferred among these are substitutions, additions and deletions which alter the properties and activities of a S. aureus protein.

The terms “polynucleotide”, and “nucleic acid” are used interchangeably to refer to a polymeric form of nucleotides of any length, either deoxyribonucleotides or ribonucleotides, or analogs thereof. The following are non-limiting examples of polynucleotides: coding or non-coding regions of a gene or gene fragment, loci (locus) defined from linkage analysis, exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component. The term “recombinant” polynucleotide means a polynucleotide of genomic, cDNA, semisynthetic, or synthetic origin which either does not occur in nature or is linked to another polynucleotide in a nonnatural arrangement. An “oligonucleotide” refers to a single stranded polynucleotide having less than about 100 nucleotides, less than about, e.g., 75, 50, 25, or 10 nucleotides.

The terms “polypeptide”, “peptide” and “protein” (if single chain) are used interchangeably herein to refer to polymers of amino acids. The polymer may be linear or branched, it may comprise modified amino acids, and it may be interrupted by non-amino acids. The terms also encompass an amino acid polymer that has been modified; for example, disulfide bond formation, glycosylation, lipidation, acetylation, phosphorylation, or any other manipulation, such as conjugation with a labeling component. As used herein the term “amino acid” refers to either natural and/or unnatural or synthetic amino acids, including glycine and both the D or L optical isomers, and amino acid analogs and peptidomimetics.

The term “sbn operon,” as used herein, refers to a group of bacterial genes comprising sbnA, sbnB, sbnC, sbnD, sbnE, sbnF, sbnG, sbnH, and sbnI that share a common promoter. The promoter element, which is upstream of the sbnA coding region, is iron-regulated. This operon, as shown herein, is responsible for the biosynthesis of a siderophore referred to as staphylobactin. The nucleotide sequence for the sbn operon has been deposited in Genbank and assigned accession no. AY251022. Each coding region of the sbn operon encodes a protein required for the biosynthesis of the staphylobactin siderophore. As such, sbnA encodes a putative cysteine synthase, sbnB encodes a putative ornithine cyclodeaminase, sbnC encodes a putative IucC homolog for aerobactin biosynthesis, sbnD encodes a putative efflux protein, sbnE encodes a siderophore biosynthesis protein, sbnF encodes a putative hydroxamate biosynthesis protein, sbnG encodes an putative hydroxamate biosynthesis protein, sbnH encodes a putative ornithine or diaminopimelate decarboxylase, and sbnI encodes an unknown protein.

The terms “sbn nucleotide”, “sbn nucleic acid”, or “sbn gene” refer to sbnA, sbnB, sbnC, sbnD, sbnE, sbnF, sbnG, sbnH, and sbnI nucleic acids.

The terms “sbn protein” or “sbn polypeptide” refer to the products of each gene of the sbn operon, i.e., “SbnA”, “SbnB”, “SbnC”, “SbnC”, “SbnD”, “SbnE”, “SbnF”, “SbnG”, “SbnH” and “SbnI,” and encompasses fragments and portions thereof and biologically active fragments or portions thereof. In exemplary embodiment, the sbn polypeptides described herein participate in the biosynthesis of staphylobactin. Specific functions of Sbn polypeptides are further described below.

The term “Sbn deficient strain” refers to a bacterial strain that does not express at least one Sbn protein.

The term “staphylobactin” refers to the iron-siderophore that is synthesized by the sbn operon and transported into cell by the SirABC iron-siderophore transport system.

The term “small molecule” refers to a compound, which has a molecular weight of less than about 5 kD, less than about 2.5 kD, less than about 1.5 kD, or less than about 0.9 kD. Small molecules may be, for example, nucleic acids, peptides, polypeptides, peptide nucleic acids, peptidomimetics, carbohydrates, lipids or other organic (carbon containing) or inorganic molecules. Many pharmaceutical companies have extensive libraries of chemical and/or biological mixtures, often fungal, bacterial, or algal extracts, which can be screened with any of the assays of the invention. The term “small organic molecule” refers to a small molecule that is often identified as being an organic or medicinal compound, and does not include molecules that are exclusively nucleic acids, peptides or polypeptides.

The term “specifically hybridizes” refers to detectable and specific nucleic acid binding. Polynucleotides, oligonucleotides and nucleic acids of the invention selectively hybridize to nucleic acid strands under hybridization and wash conditions that minimize appreciable amounts of detectable binding to nonspecific nucleic acids. Stringent conditions may be used to achieve selective hybridization conditions as known in the art and discussed herein. Generally, the nucleic acid sequence homology between the polynucleotides, oligonucleotides, and nucleic acids of the invention and a nucleic acid sequence of interest will be at least 30%, 40%, 50%, 60%, 70%, 80%, 85%, 90%, 95%, 98%, 99%, or more. In certain instances, hybridization and washing conditions are performed under stringent conditions according to conventional hybridization procedures and as described further herein.

The terms “stringent conditions” or “stringent hybridization conditions” refer to conditions which promote specific hybridization between two complementary polynucleotide strands so as to form a duplex. Stringent conditions may be selected to be about 5° C. lower than the thermal melting point (Tm) for a given polynucleotide duplex at a defined ionic strength and pH. The length of the complementary polynucleotide strands and their GC content will determine the Tm of the duplex; and thus the hybridization conditions necessary for obtaining a desired specificity of hybridization. The Tm is the temperature (under defined ionic strength and pH) at which 50% of a polynucleotide sequence hybridizes to a perfectly matched complementary strand. In certain cases it may be desirable to increase the stringency of the hybridization conditions to be about equal to the Tm for a particular duplex.

A variety of techniques for estimating the Tm are available. Typically, G-C base pairs in a duplex are estimated to contribute about 3° C. to the Tm, while A-T base pairs are estimated to contribute about 2° C., up to a theoretical maximum of about 80-100° C. However, more sophisticated models of Tm are available in which G-C stacking interactions, solvent effects, the desired assay temperature and the like are taken into account. For example, probes can be designed to have a dissociation temperature (Td) of approximately 60° C., using the formula: Td=(((((3×#GC)+(2×#AT))×37)−562)/#bp)−5; where #GC, #AT, and #bp are the number of guanine-cytosine base pairs, the number of adenine-thymine base pairs, and the number of total base pairs, respectively, involved in the formation of the duplex.

Hybridization may be carried out in 5×SSC, 4×SSC, 3×SSC, 2×SSC, 1×SSC or 0.2×SSC for at least about 1 hour, 2 hours, 5 hours, 12 hours, or 24 hours. The temperature of the hybridization may be increased to adjust the stringency of the reaction, for example, from about 25° C. (room temperature), to about 45° C., 50° C., 55° C., 60° C., or 65° C. The hybridization reaction may also include another agent affecting the stringency, for example, hybridization conducted in the presence of 50% formamide increases the stringency of hybridization at a defined temperature.

The hybridization reaction may be followed by a single wash step, or two or more wash steps, which may be at the same or a different salinity and temperature. For example, the temperature of the wash may be increased to adjust the stringency from about 25° C. (room temperature), to about 45° C., 50° C., 55° C., 60° C., 65° C., or higher. The wash step may be conducted in the presence of a detergent, e.g., 0.1 or 0.2% SDS. For example, hybridization may be followed by two wash steps at 65° C. each for about 0.20 minutes in 2×SSC, 0.1% SDS, and optionally two additional wash steps at 65° C. each for about 20 minutes in 0.2×SSC, 0.1% SDS.

Exemplary stringent hybridization conditions include overnight hybridization at 65° C. in a solution comprising, or consisting of, 50% formamide; 10×Denhardt (0.2% Ficoll, 0.2% Polyvinylpyrrolidone, 0.2% bovine serum albumin) and 200 μg/ml of denatured carrier DNA, e.g., sheared salmon sperm DNA, followed by two wash steps at 65° C. each for about 20 minutes in 2×SSC, 0.1% SDS, and two wash steps at 65° C. each for about 20 minutes in 0.2×SSC, 0.1% SDS.

Hybridization may consist of hybridizing two nucleic acids in solution, or a nucleic acid in solution to a nucleic acid attached to a solid support, e.g., a filter. When one nucleic acid is on a solid support, a prehybridization step may be conducted prior to hybridization. Prehybridization may be carried out for at least about 1 hour, 3 hours or 10 hours in the same solution and at the same temperature as the hybridization solution (without the complementary polynucleotide strand).

Appropriate stringency conditions are known to those skilled in the art or may be determined experimentally by the skilled artisan. See, for example, Current Protocols in Molecular Biology, John Wiley & Sons, N.Y. (1989), 6.3.1-12.3.6; Sambrook et al., 1989, Molecular Cloning, A Laboratory Manual, Cold Spring Harbor Press; N.Y.; S. Agrawal (ed.) Methods in Molecular Biology, volume 20; Tijssen (1993) Laboratory Techniques in biochemistry and molecular biology-hybridization with nucleic acid probes, e.g., part I chapter 2 “Overview of principles of hybridization and the strategy of nucleic acid probe assays”, Elsevier, New York; and Tibanyenda, N. et al., Eur. J. Biochem. 139:19 (1984) and Ebel, S. et al., Biochem. 31:12083 (1992).

The term “substantially homologous” when used in connection with a nucleic acid or amino acid sequences, refers to sequences which are substantially identical to or similar in sequence with each other, giving rise to a homology of conformation and thus to retention, to a useful degree, of one or more biological (including immunological) activities. The term is not intended to imply a common evolution of the sequences.

A “subject” refers to a male or female mammal, including humans.

A “vector” is a self-replicating nucleic acid molecule that transfers an inserted nucleic acid molecule into and/or between host cells. The term includes vectors that function primarily for insertion of a nucleic acid molecule into a cell, replication of vectors that function primarily for the replication of nucleic acid, and expression vectors that function for transcription and/or translation of the DNA or RNA. Also included are vectors that provide more than one of the above functions. As used herein, “expression vectors” are defined as polynucleotides which, when introduced into an appropriate host cell, can be transcribed and translated into a polypeptide(s). An “expression system” usually connotes a suitable host cell comprised of an expression vector that can function to yield a desired expression product.

### 3. Sbn Genes

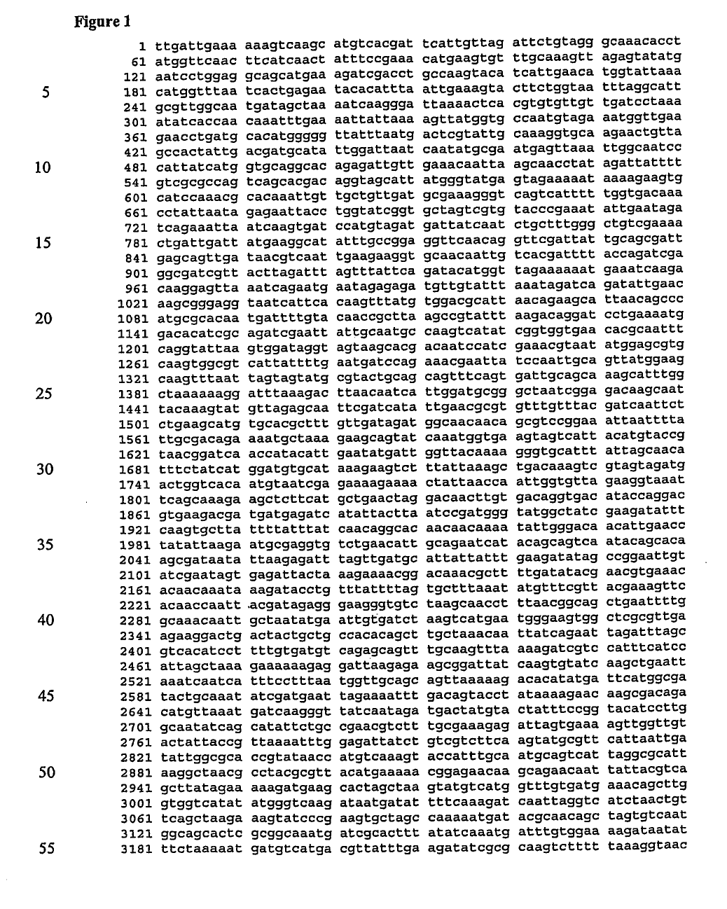

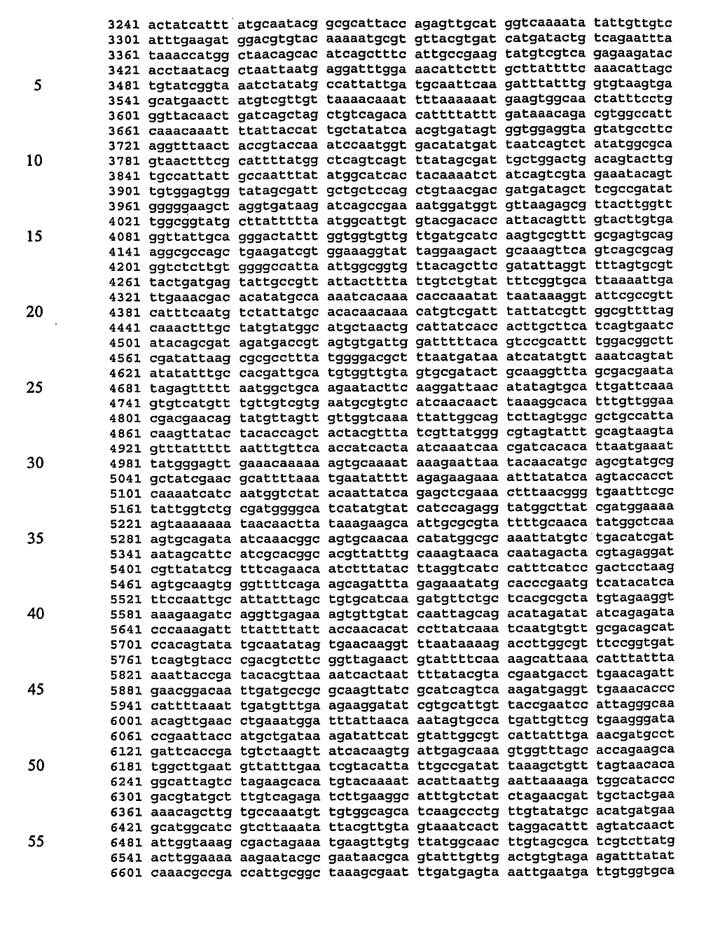

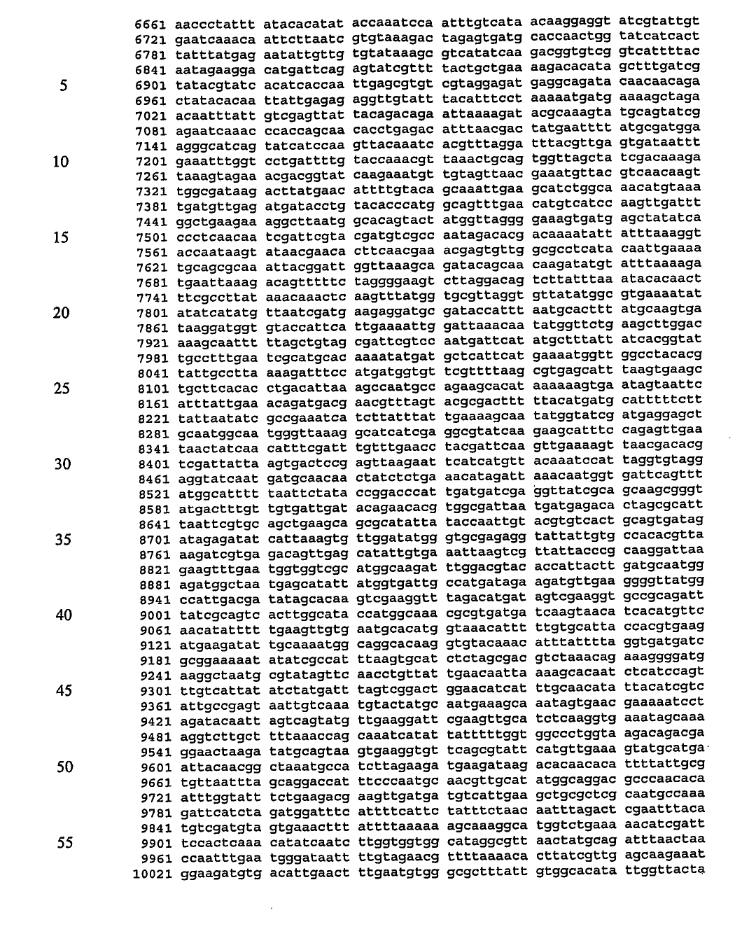

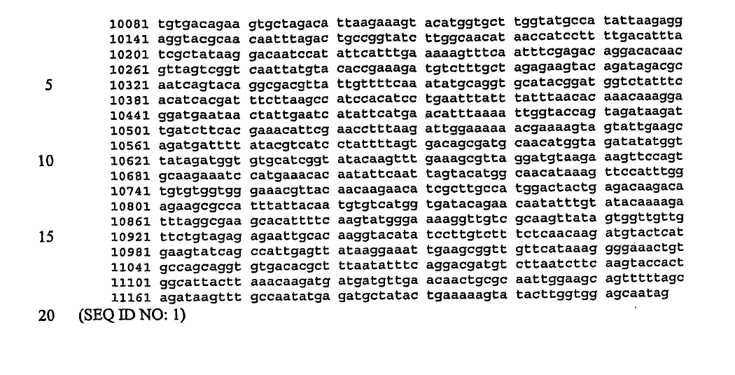

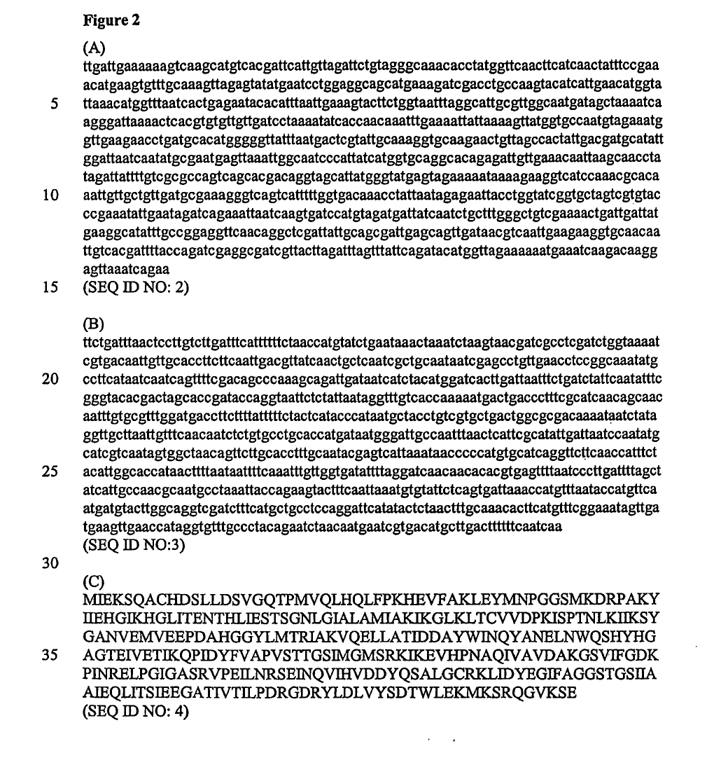

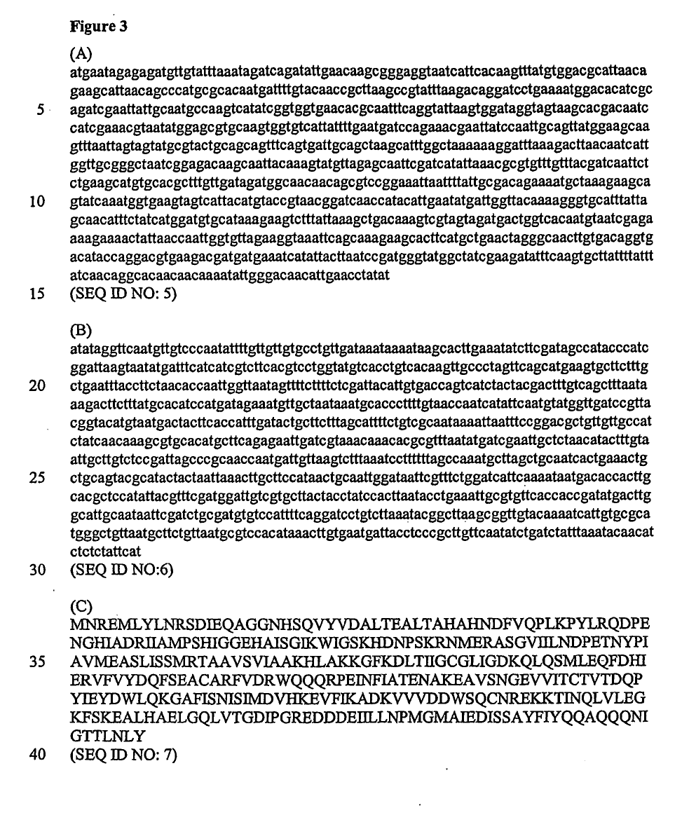

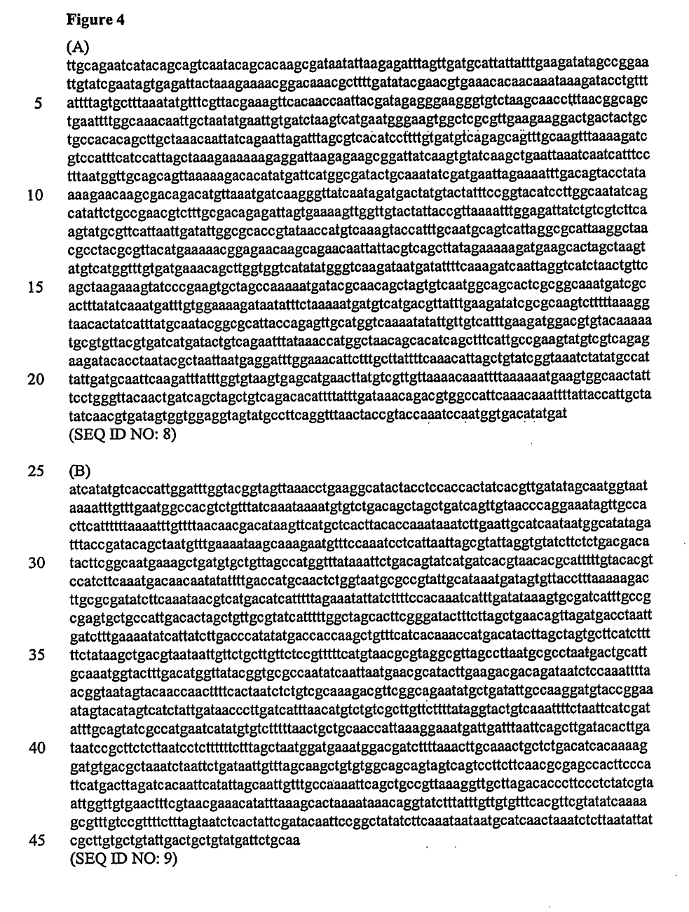

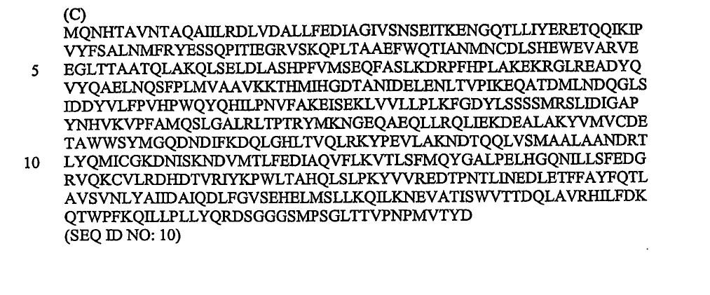

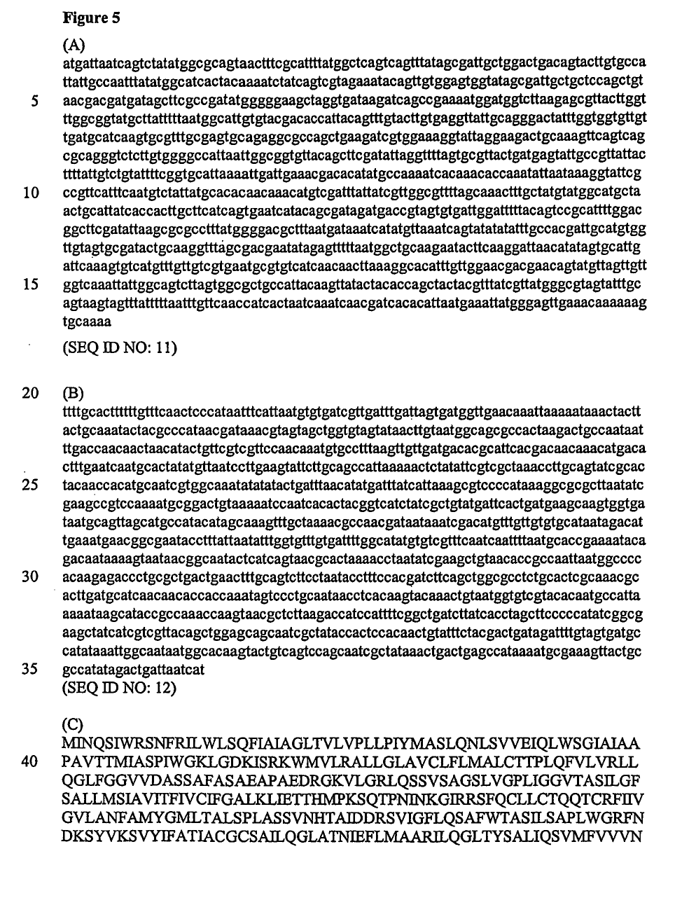

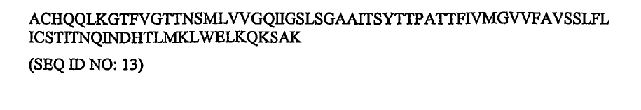

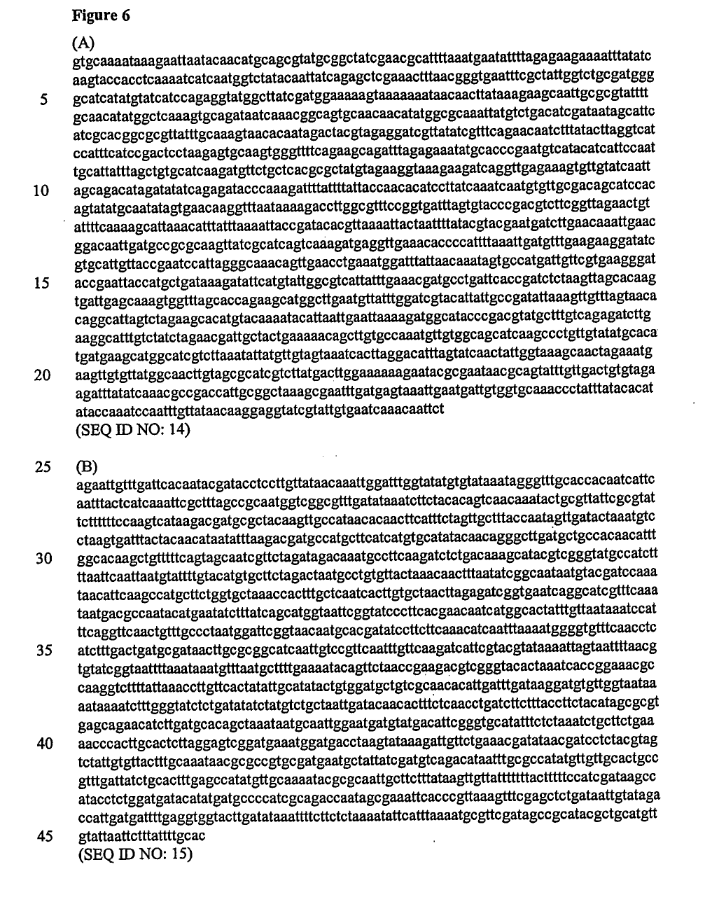

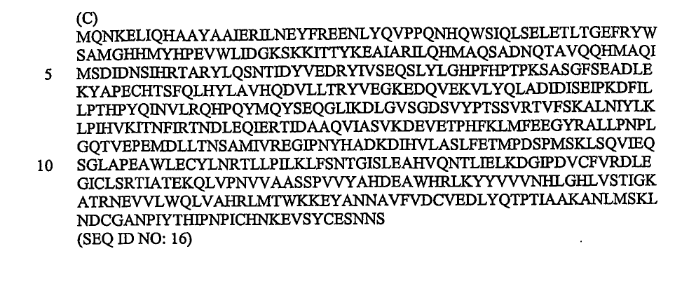

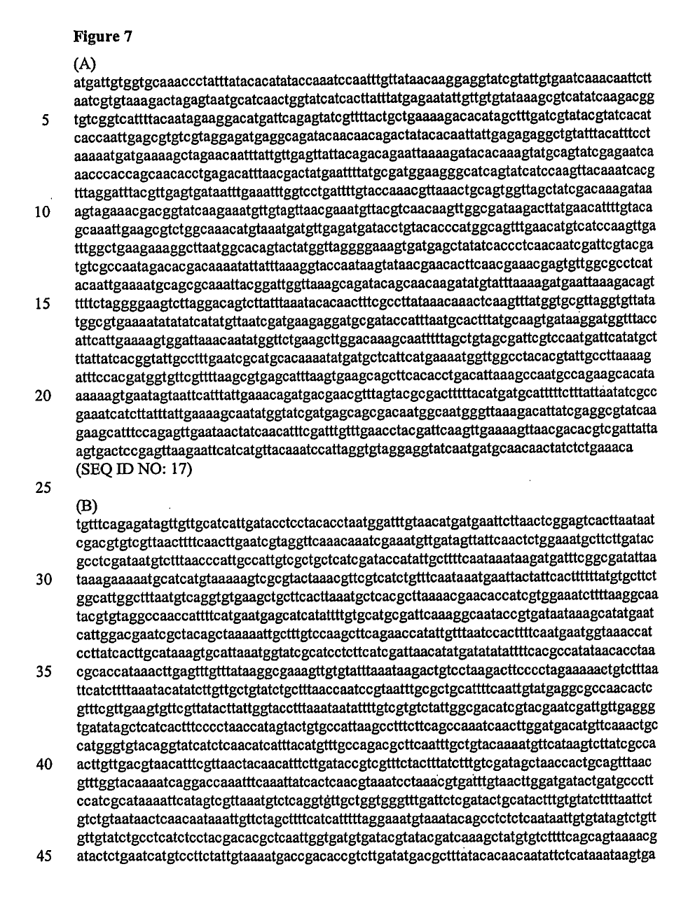

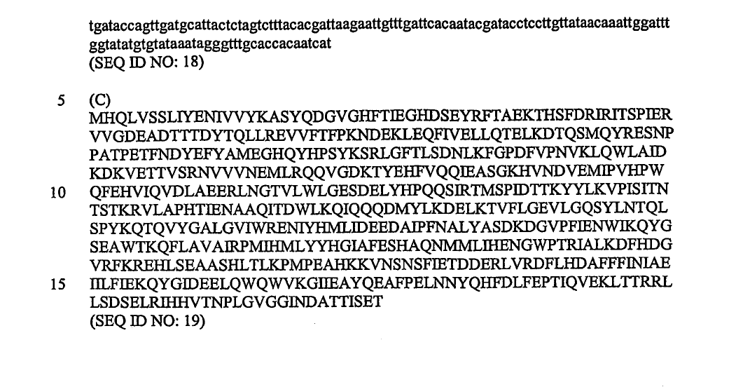

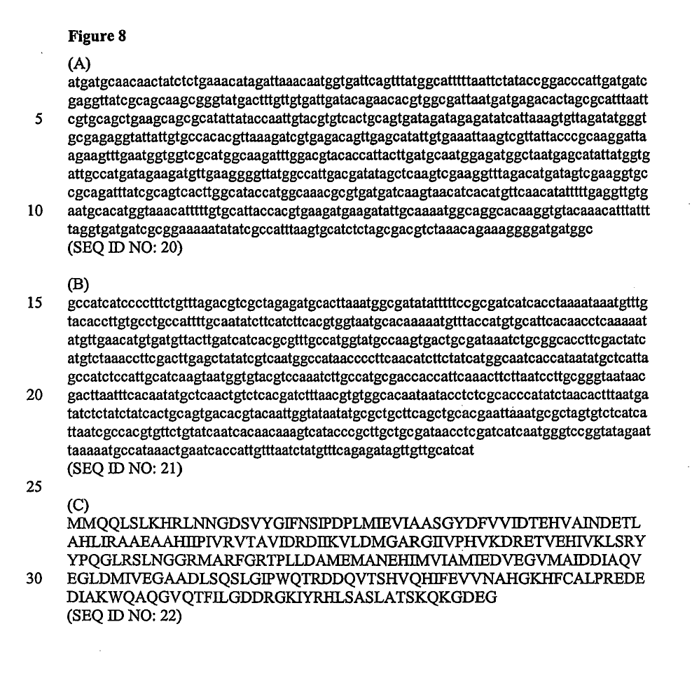

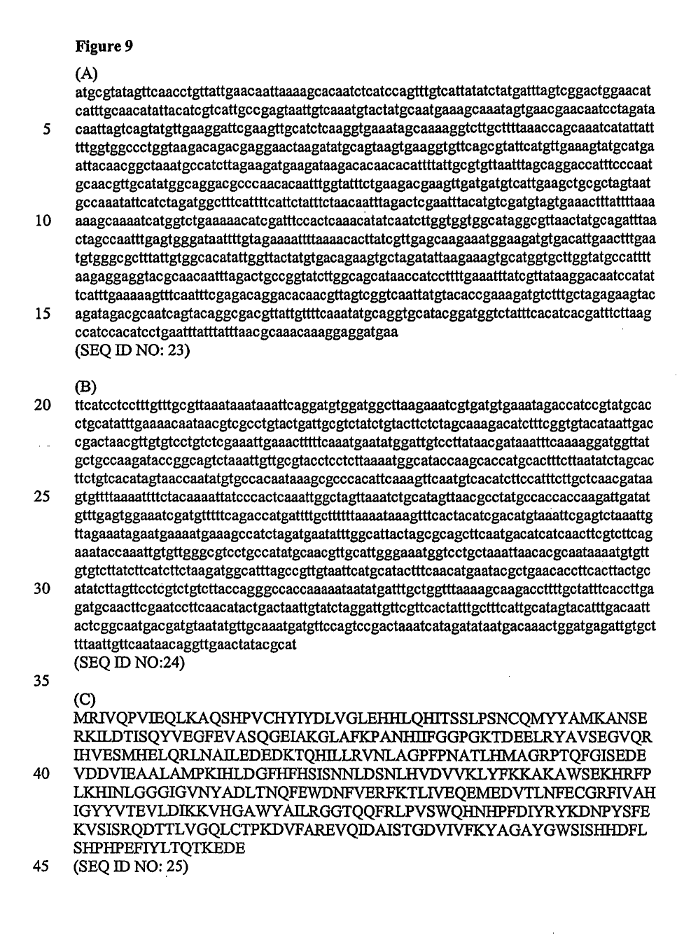

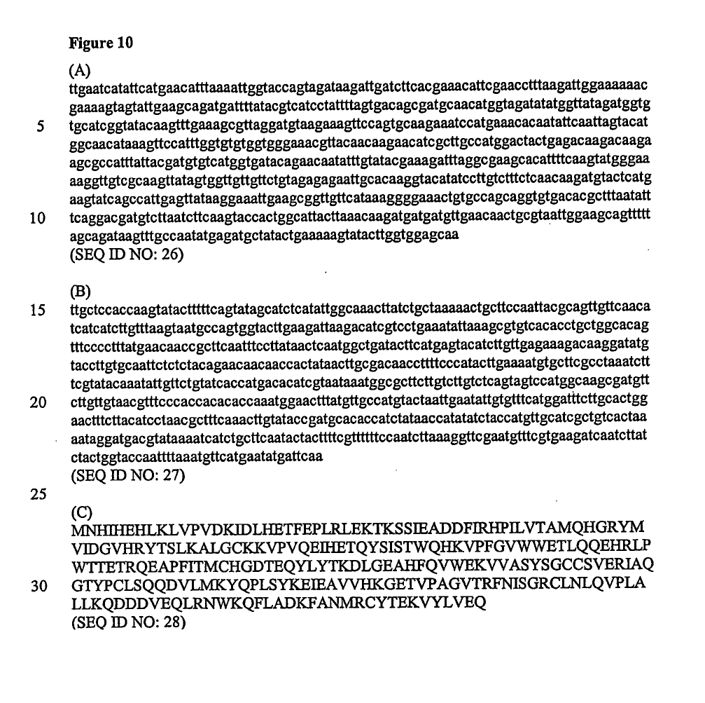

The present invention features nucleic acid molecules which comprise a siderophore biosynthetic gene cluster in S. aureus referred to herein as the sbn operon (FIG. 1; SEQ ID NO:1). Nine genes comprise the sbn operon and are referred to herein as sbnA, sbnB, sbnC, sbnD, sbnE, sbnF, sbnG, sbnH, and sbnI (FIGS. 2-10; SEQ ID NOs: 2, 4, 6, 8, 10, 12, 14, 16, and 18).

Nucleic acids of the present invention may also comprise, consist of or consist essentially of any of the sbn nucleotide sequences described herein, the full complement or mutants thereof. Yet other nucleic acids comprise, consist of or consist essentially of an nucleotide sequence that has at least about 70%, 80%, 90%, 95%, 98% or 99% identity or homology with a sbn gene or the complement thereof. Substantially homologous sequences may be identified using stringent hybridization conditions.

Isolated nucleic acids which differ from the nucleic acids of the invention due to degeneracy in the genetic code are also within the scope of the invention. For example, a number of amino acids are designated by more than one triplet. Codons that specify the same amino acid, or synonyms (for example, CAU and CAC are synonyms for histidine) may result in “silent” mutations which do not affect the amino acid sequence of the protein. However, it is expected that DNA sequence polymorphisms that do lead to changes in the amino acid sequences of the polypeptides of the invention will exist. One skilled in the art will appreciate that these variations in one or more nucleotides (from less than 1% up to about 3 or 5% or possibly more of the nucleotides) of the nucleic acids encoding a particular protein of the invention may exist among a given species due to natural allelic variation. Any and all such nucleotide variations and resulting amino acid polymorphisms are within the scope of this invention.

Nucleic acids encoding proteins which have amino acid sequences evolutionarily related to a polypeptide disclosed herein are provided, wherein “evolutionarily related to”, refers to proteins having different amino acid sequences which have arisen naturally (e.g. by allelic variance or by differential splicing), as well as mutational variants of the proteins of the invention which are derived, for example, by combinatorial mutagenesis.

Fragments of the polynucleotides of the invention encoding a biologically active portion of the subject polypeptides are also provided. As used herein, a fragment of a nucleic acid encoding an active portion of a polypeptide disclosed herein refers to a nucleotide sequence having fewer nucleotides than the nucleotide sequence encoding the full length amino acid sequence of a polypeptide of the invention, and which encodes a given polypeptide that retains at least a portion of a biological activity of the full-length Sbn protein as defined herein, or alternatively, which is functional as a modulator of the biological activity of the full-length protein. For example, such fragments include a polypeptide containing a domain of the full-length protein from which the polypeptide is derived that mediates the interaction of the protein with another molecule (e.g., polypeptide, DNA, RNA, etc.).

Nucleic acids provided herein may also contain linker sequences, modified restriction endonuclease sites and other sequences useful for molecular cloning, expression or purification of such recombinant polypeptides.

A nucleic acid encoding a Sbn polypeptide provided herein may be obtained from mRNA or genomic DNA from any organism in accordance with protocols described herein, as well as those generally known to those skilled in the art. A cDNA encoding a polypeptide of the invention, for example, may be obtained by isolating total mRNA from an organism, for example, a bacteria, virus, mammal, etc. Double stranded cDNAs may then be prepared from the total mRNA, and subsequently inserted into a suitable plasmid or bacteriophage vector using any one of a number of known techniques. A gene encoding a polypeptide of the invention may also be cloned using established polymerase chain reaction techniques in accordance with the nucleotide sequence information provided by the invention. In one aspect, methods for amplification of a nucleic acid of the invention, or a fragment thereof may comprise: (a) providing a pair of single stranded oligonucleotides, each of which is at least eight nucleotides in length, complementary to sequences of a nucleic acid of the invention, and wherein the sequences to which the oligonucleotides are complementary are at least ten nucleotides apart; and (b) contacting the oligonucleotides with a sample comprising a nucleic acid comprising the nucleic acid of the invention under conditions which permit amplification of the region located between the pair of oligonucleotides, thereby amplifying the nucleic acid. The present invention also features recombinant vectors, which include isolated genes, which encode proteins required for staphylobactin biosynthesis (i.e., sbnA, sbnB, sbnC, sbnD, sbnE, sbnF, sbnG, sbnH, and sbnI nucleic acids), host cells containing the recombinant vectors and methods of producing the encoded S. aureus polypeptides.

Appropriate vectors may be introduced into host cells using well known techniques such as infection, transduction, transfection, transfection, electroporation and transformation. The vector may be, for example, a phage, plasmid, viral or retroviral vector. Retroviral vectors may be replication competent or replication defective. In the latter case, viral propagation generally will occur only in complementing host cells.

The vector may contain a selectable marker for propagation in a host. Generally, a plasmid vector is introduced in a precipitate, such as a calcium phosphate precipitate, or in a complex with a charged lipid. If the vector is a virus, it may be packaged in vitro using an appropriate packaging cell line and then transduced into host cells.

Preferred vectors comprise cis-acting control regions to the polynucleotide of interest. Appropriate trans-acting factors may be supplied by the host, supplied by a complementing vector or supplied by the vector itself upon introduction into the host.

In certain embodiments, the vectors provide for specific expression, which may be inducible and/or cell type-specific. Particularly preferred among such vectors are those inducible by environmental factors that are easy to manipulate, such as temperature and nutrient additives.

Expression vectors useful in the present invention include chromosomal-, episomal- and virus-derived vectors, e.g., vectors derived from bacterial plasmids, bacteriophage, yeast episomes, yeast chromosomal elements, viruses such as baculoviruses, papova viruses, vaccinia viruses, adenoviruses, fowl pox viruses, pseudorabies viruses and retroviruses, and vectors derived from combinations thereof, such as cosmids and phagemids.

The DNA insert should be operatively linked to an appropriate promoter, such as the phage lambda PL promoter, the E. coli lac, trp and tac promoters, the SV40 early and late promoters and promoters of retroviral LTRs, to name a few. Other suitable promoters will be known to the skilled artisan. The expression constructs will further contain sites for transcription initiation, termination and, in the transcribed region, a ribosome binding site for translation. The coding portion of the mature transcripts expressed by the constructs will preferably include a translation initiating site at the beginning and a termination codon (UAA, UGA or UAG) appropriately positioned at the end of the polypeptide to be translated.

As indicated, the expression vectors will preferably include at least one selectable marker. Such markers include dihydrofolate reductase or neomycin resistance for eukaryotic cell culture and tetracycline, kanamycin, or ampicillin resistance genes for culturing in E. coli and other bacteria. Representative examples of appropriate hosts include, but are not limited to, bacterial cells, such as E. coli, Streptomyces and Salmonella typhimurium cells; fungal cells, such as yeast cells; insect cells such as Drosophila S2 and Sf9 cells; animal cells such as CHO, COS and Bowes melanoma cells; and plant cells. Appropriate culture mediums and conditions for the above-described host cells are known in the art.

Among vectors preferred for use in bacteria include pQE70, pQE60 and pQE9, pQE10 available from Qiagen; pBS vectors, Phagescript vectors, Bluescript vectors, pNH8A, pNH16a, pNH18A, pNH46A available from Stratagene; pET series of vectors available from Novagen; and ptrc99a, pKK223-3, pKK233-3, pDR540, pRIT5 available from Pharmacia. Among preferred eukaryotic vectors are pWLNEO, pSV2CAT, pOG44, pXT1 and pSG available from Stratagene; and pSVK3, pBPV, pMSG and pSVL available from Pharmacia. Other suitable vectors will be readily apparent to the skilled artisan.

Among known bacterial promoters suitable for use in the present invention include the E. coli lacI and lacZ promoters, the T3, T5 and T7 promoters, the gpt promoter, the lambda PR and PL promoters, the trp promoter and the xyI/tet chimeric promoter. Suitable eukaryotic promoters include the CMV immediate early promoter, the HSV thymidine kinase promoter, the early and late SV40 promoters, the promoters of retroviral LTRs, such as those of the Rous sarcoma virus (RSV), and metallothionein promoters, such as the mouse metallothionein-I promoter.

Introduction of the construct into the host cell can be effected by calcium phosphate transfection, DEAE-dextran mediated transfection, cationic lipid-mediated transfection, electroporation, transduction, infection or other methods. Such methods are described in many standard laboratory manuals (for example, Davis, et al., Basic Methods in Molecular Biology (1986)).

Transcription of DNA encoding the polypeptides of the present invention by higher eukaryotes may be increased by inserting an enhancer sequence into the vector. Enhancers are cis-acting elements of DNA, usually about from 10 to 300 nucleotides that act to increase transcriptional activity of a promoter in a given host cell-type. Examples of enhancers include the SV40 enhancer, which is located on the late side of the replication origin at nucleotides 100 to 270, the cytomegalovirus early promoter enhancer, the polyoma enhancer on the late side of the replication origin; and adenovirus enhancers.

For secretion of the translated polypeptide into the lumen of the endoplasmic reticulum, into the periplasmic space or into the extracellular environment, appropriate secretion signals may be incorporated into the expressed polypeptide, for example, the amino acid sequence KDEL. The signals may be endogenous to the polypeptide or they may be heterologous signals.

Coding sequences for a polypeptide of interest may be incorporated as a part of a fusion gene including a nucleotide sequence encoding a different polypeptide. The present invention contemplates an isolated nucleic acid comprising a nucleic acid of the invention and at least one heterologous sequence encoding a heterologous peptide linked in frame to the nucleotide sequence of the nucleic acid of the invention so as to encode a fusion protein comprising the heterologous polypeptide. The heterologous polypeptide may be fused to (a) the C-terminus of the polypeptide encoded by the nucleic acid of the invention, (b) the N-terminus of the polypeptide, or (c) the C-terminus and the N-terminus of the polypeptide. In certain instances, the heterologous sequence encodes a polypeptide permitting the detection, isolation, solubilization and/or stabilization of the polypeptide to which it is fused. In still other embodiments, the heterologous sequence encodes a polypeptide selected from the group consisting of a polyHis tag, myc, HA, GST, protein A, protein G, calmodulin-binding peptide, thioredoxin, maltose-binding protein, poly arginine, poly His-Asp, FLAG, a portion of an immunoglobulin protein, and a transcytosis peptide.

Fusion expression systems can be useful when it is desirable to produce an immunogenic fragment of a polypeptide of the invention. For example, the VP6 capsid protein of rotavirus may be used as an immunologic carrier protein for portions of polypeptide, either in the monomeric form or in the form of a viral particle. The nucleic acid sequences corresponding to the portion of a polypeptide of the invention to which antibodies are to be raised may be incorporated into a fusion gene construct which includes coding sequences for a late vaccinia virus structural protein to produce a set of recombinant viruses expressing fusion proteins comprising a portion of the protein as part of the virion. The Hepatitis B surface antigen may also be utilized in this role as well. Similarly, chimeric constructs coding for fusion proteins containing a portion of a polypeptide of the invention and the poliovirus capsid protein may be created to enhance immunogenicity (see, for example, EP Publication NO: 0259149; and Evans et al., (1989) Nature 339:385; Huang et al., (1988) J. Virol. 62:3855; and Schlienger et al, (1992) J. Virol. 66:2).

Fusion proteins may facilitate the expression and/or purification of proteins. For example, a polypeptide of the invention may be generated as a glutathione-S-transferase (GST) fusion protein. Such GST fusion proteins may be used to simplify purification of a polypeptide of the invention, such as through the use of glutathione-derivatized matrices (see, for example, Current Protocols in Molecular Biology, eds. Ausubel et al., (N.Y.: John Wiley & Sons, 1991)). In another embodiment, a fusion gene coding for a purification leader sequence, such as a poly-(His)/enterokinase cleavage site sequence at the N-terminus of the desired portion of the recombinant protein, may allow purification of the expressed fusion protein by affinity chromatography using a Ni²⁺ metal resin. The purification leader sequence may then be subsequently removed by treatment with enterokinase to provide the purified protein (e.g., see Hochuli et al., (1987) J. Chromatography 411: 177; and Janknecht et al., PNAS USA 88:8972).

Techniques for making fusion genes are well known. Essentially, the joining of various DNA fragments coding for different polypeptide sequences is performed in accordance with conventional techniques, employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene may be synthesized by conventional techniques including automated DNA synthesizers. Alternatively, PCR amplification of gene fragments may be carried out using anchor primers which give rise to complementary overhangs between two consecutive gene fragments which may subsequently be annealed to generate a chimeric gene sequence (see, for example, Current Protocols in Molecular Biology, eds. Ausubel et al., John Wiley & Sons: 1992).

In other embodiments, nucleic acids of the invention may be immobilized onto a solid surface, including, plates, microtiter plates, slides, beads, particles, spheres, films, strands, precipitates, gels, sheets, tubing, containers, capillaries, pads, slices, etc. The nucleic acids of the invention may be immobilized onto a chip as part of an array. The array may comprise one or more polynucleotides of the invention as described herein. In one embodiment, the chip comprises one or more polynucleotides of the invention as part of an array of polynucleotide sequences.

Another aspect relates to the use of nucleic acids of the invention in “antisense therapy”. As used herein, antisense therapy refers to administration or in situ generation of oligonucleotide probes or their derivatives which specifically hybridize or otherwise bind under cellular conditions with the cellular mRNA and/or genomic DNA encoding one of the polypeptides of the invention so as to inhibit expression of that polypeptide, e.g., by inhibiting transcription and/or translation. The binding may be by conventional base pair complementarity, or, for example, in the case of binding to DNA duplexes, through specific interactions in the major groove of the double helix. In general, antisense therapy refers to the range of techniques generally employed in the art; and includes any therapy which relies on specific binding to oligonucleotide sequences.

The oligonucleotide may be conjugated to another molecule, e.g., a peptide, hybridization triggered cross-linking agent transport agent, hybridization-triggered cleavage agent, etc. An antisense molecule can be a “peptide nucleic acid” (PNA). PNA refers to an antisense molecule or anti-gene agent which comprises an oligonucleotide of at least about 5 nucleotides in length linked to a peptide backbone of amino acid residues ending in lysine. The terminal lysine confers solubility to the composition. PNAs preferentially bind complementary single stranded DNA or RNA and stop transcript elongation, and may be pegylated to extend their lifespan in the cell.

An antisense construct of the present invention may be delivered, for example, as an expression plasmid which, when transcribed in the cell, produces RNA which is complementary to at least a unique portion of the mRNA which encodes a polypeptide of the invention. Alternatively, the antisense construct may be an oligonucleotide probe which is generated ex vivo and which, when introduced into the cell causes inhibition of expression by hybridizing with the mRNA and/or genomic sequences encoding a polypeptide of the invention. Such oligonucleotide probes may be modified oligonucleotides which are resistant to endogenous nucleases, e.g., exonucleases and/or endonucleases, and are therefore stable in vivo. Exemplary nucleic acid molecules for use as antisense oligonucleotides are phosphoramidate, phosphothioate and methylphosphonate analogs of DNA (see also U.S. Pat. Nos. 5,176,996; 5,264,564; and 5,256,775). Additionally, general approaches to constructing oligomers useful in antisense therapy have been reviewed, for example, by van der Krol et al., (1988) Biotechniques 6:958-976; and Stein et al., (1988) Cancer Res 48:2659-2668.

In a further aspect, double stranded small interfering RNAs (siRNAs), and methods for administering the same are provided. siRNAs decrease or block gene expression. While not wishing to be bound by theory, it is generally thought that siRNAs inhibit gene expression by mediating sequence specific mRNA degradation. RNA interference (RNAi) is the process of sequence-specific, post-transcriptional gene silencing, particularly in animals and plants, initiated by double-stranded RNA (dsRNA) that is homologous in sequence to the silenced gene (Elbashir et al. Nature 2001; 411(6836): 494-8). Accordingly, it is understood that siRNAs and long dsRNAs having substantial sequence identity to all or a portion of a polynucleotide of the present invention may be used to inhibit the expression of a nucleic acid of the invention.

Alternatively, siRNAs that decrease or block the expression the Sir or FhuC polypeptides described herein may be determined by testing a plurality of siRNA constructs against the target gene. Such siRNAs against a target gene may be chemically synthesized. The nucleotide sequences of the individual RNA strands are selected such that the strand has a region of complementarity to the target gene to be inhibited (i.e., the complementary RNA strand comprises a nucleotide sequence that is complementary to a region of an mRNA transcript that is formed during expression of the target gene, or its processing products, or a region of a (+) strand virus). The step of synthesizing the RNA strand may involve solid-phase synthesis, wherein individual nucleotides are joined end to end through the formation of internucleotide 3′-5′ phosphodiester bonds in consecutive synthesis cycles.

Provided herein are siRNA molecules comprising a nucleotide sequence consisting essentially of a sequence of a sbn nucleic acid as described herein. An siRNA molecule may comprise two strands, each strand comprising a nucleotide sequence that is at least essentially complementary to each other, one of which corresponds essentially to a sequence of a target gene. The sequence that corresponds essentially to a sequence of a target gene is referred to as the “sense target sequence” and the sequence that is essentially complementary thereto is referred to as the “antisense target sequence” of the siRNA. The sense and antisense target sequences may be from about 15 to about 30 consecutive nucleotides long; from about 19 to about 25 consecutive nucleotides; from about 19 to 23 consecutive nucleotides or about 19, 20, 21, 22 or 23 nucleotides long. The length of the sense and antisense sequences is determined so that an siRNA having sense and antisense target sequences of that length is capable of inhibiting expression of a target gene, preferably without significantly inducing a host interferon response.

SiRNA target sequences may be predicted using any of the algorithms provided on the world wide web at the mmcmanus with the extension web.mit.edu/mmcmanus/www/home1.2files/siRNAs.

The sense target sequence may be essentially or substantially identical to the coding or a non-coding portion, or combination thereof, of a target nucleic acid. For example, the sense target sequence may be essentially complementary to the 5′ or 3′ untranslated region, promoter, intron or exon of a target nucleic acid or complement thereof. It can also be essentially complementary to a region encompassing the border between two such gene regions.

The nucleotide base composition of the sense target sequence can be about 50% adenines (As) and thymidines (Ts) and 50% cytidines (Cs) and guanosines (Gs). Alternatively, the base composition can be at least 50% Cs/Gs, e.g., about 60%, 70% or 80% of Cs/Gs. Accordingly, the choice of sense target sequence may be based on nucleotide base composition. Regarding the accessibility of target nucleic acids by siRNAs, such can be determined, e.g., as described in Lee et al. (2002) Nature Biotech. 19:500. This approach involves the use of oligonucleotides that are complementary to the target nucleic acids as probes to determine substrate accessibility, e.g., in cell extracts. After forming a duplex with the oligonucleotide probe, the substrate becomes susceptible to RNase H. Therefore, the degree of RNase H sensitivity to a given probe as determined, e.g., by PCR, reflects the accessibility of the chosen site, and may be of predictive value for how well a corresponding siRNA would perform in inhibiting transcription from this target gene. One may also use algorithms identifying primers for polymerase chain reaction (PCR) assays or for identifying antisense oligonucleotides for identifying first target sequences.

The sense and antisense target sequences are preferably sufficiently complementary, such that an siRNA comprising both sequences is able to inhibit expression of the target gene, i.e., to mediate RNA interference. For example, the sequences may be sufficiently complementary to permit hybridization under the desired conditions, e.g., in a cell. Accordingly, the sense and antisense target sequences may be at least about 95%, 97%, 98%, 99% or 100% identical and may, e.g., differ in at most 5, 4, 3, 2, 1 or 0 nucleotides.

Sense and antisense target sequences are also preferably sequences that are not likely to significantly interact with sequences other, than the target nucleic acid or complement thereof. This can be confirmed by, e.g., comparing the chosen sequence to the other sequences in the genome of the target cell. Sequence comparisons can be performed according to methods known in the art, e.g., using the BLAST algorithm, further described herein. Of course, small scale experiments can also be performed to confirm that a particular first target sequence is capable of specifically inhibiting expression of a target nucleic acid and essentially not that of other genes.

siRNAs may also comprise sequences in addition to the sense and antisense sequences. For example, an siRNA may be an RNA duplex consisting of two strands of RNA, in which at least one strand has a 3′ overhang. The other strand can be blunt-ended or have an overhang. In the embodiment in which the RNA molecule is double stranded and both strands comprise an overhang, the length of the overhangs may be the same or different for each strand. In a particular embodiment, an siRNA comprises sense and antisense sequences, each of which are on one RNA strand, consisting of about 19-25 nucleotides which are paired and which have overhangs of from about 1 to about 3, particularly about 2, nucleotides on both 3′ ends of the RNA. In order to further enhance the stability of the RNA of the present invention, the 3′ overhangs can be stabilized against degradation. In one embodiment, the RNA is stabilized by including purine nucleotides, such as adenosine or guanosine nucleotides. Alternatively, substitution of pyrimidine nucleotides by modified analogues, e.g., substitution of uridine 2 nucleotide 3′ overhangs by 2′-deoxythymidine is tolerated and does not affect the efficiency of RNAi. The absence of a 2′ hydroxyl significantly may also enhance the nuclease resistance of the overhang at least in tissue culture medium. RNA strands of siRNAs may have a 5′ phosphate and a 3′ hydroxyl group.

In one embodiment, an siRNA molecule comprises two strands of RNA forming a duplex. In another embodiment, an siRNA molecule consists of one RNA strand forming a hairpin loop, wherein the sense and antisense target sequences hybridize and the sequence between the two target sequences is a spacer sequence that essentially forms the loop of the hairpin structure. The spacer sequence may be any combination of nucleotides and any length provided that two complementary oligonucleotides linked by a spacer having this sequence can form a hairpin structure, wherein at least part of the spacer forms the loop at the closed end of the hairpin. For example, the spacer sequence can be from about 3 to about 30 nucleotides; from about 3 to about 20 nucleotides; from about 5 to about 15 nucleotides; from about 5 to about 10 nucleotides; or from about 3 to about 9 nucleotides. The sequence can be any sequence, provided that it does not interfere with the formation of a hairpin structure. In particular, the spacer sequence is preferably not a sequence having any significant homology to the first or the second target sequence, since this might interfere with the formation of a hairpin structure. The spacer sequence is also preferably not similar to other sequences, e.g., genomic sequences of the cell into which the nucleic acid will be introduced, since this may result in undesirable effects in the cell.

A person of skill in the art will understand that when referring to a nucleic acid, e.g., an RNA, the RNA may comprise or consist of naturally occurring nucleotides or of nucleotide derivatives that provide, e.g., more stability to the nucleic acid. Any derivative is permitted provided that the nucleic acid is capable of functioning in the desired fashion. For example, an siRNA may comprise nucleotide derivatives provided that the siRNA is still capable of inhibiting expression of the target gene.

For example, siRNAs may include one or more modified base and/or a backbone modified for stability or for other reasons. For example, the phosphodiester linkages of natural RNA may be modified to include at least one of a nitrogen or sulphur heteroatom. Moreover, siRNA comprising unusual bases, such as inosine, or modified bases, such as tritylated bases, to name just two examples, can be used in the invention. It will be appreciated that a great variety of modifications have been made to RNA that serve many useful purposes known to those of skill in the art. The term siRNA as it is employed herein embraces such chemically, enzymatically or metabolically modified forms of siRNA, provided that it is derived from an endogenous template.

There is no limitation on the manner in which an siRNA may be synthesised. Thus, it may synthesized in vitro or in vivo, using manual and/or automated procedures. In vitro synthesis may be chemical or enzymatic, for example using cloned RNA polymerase (e.g., T3, T7, SP6) for transcription of a DNA (or cDNA) template, or a mixture of both. SiRNAs may also be prepared by synthesizing each of the two strands, e.g., chemically, and hybridizing the two strands to form a duplex. In vivo, the siRNA may be synthesized using recombinant techniques well known in the art (see e.g., Sambrook, et al., Molecular Cloning: A Laboratory Manual, Second Edition (1989); DNA Cloning, Volumes I and II (D. N Glover ed. 1985); Oligonucleotide Synthesis (M. J. Gait ed, 1984); Nucleic Acid Hybridisation (B. D. Hames & S. J. Higgins eds. 1984); Transcription and Translation (B. D. Hames & S. J. Higgins eds. 1984); Animal Cell Culture (R. I. Freshney ed. 1986); Immobilised Cells and Enzymes (IRL Press, 1986); B. Perbal, A Practical Guide to Molecular Cloning (1984); the series, Methods in Enzymology (Academic Press, Inc.); Gene Transfer Vectors for Mammalian Cells (J. H. Miller and M. P. Calos eds. 1987, Cold Spring Harbor Laboratory), Methods in Enzymology Vol. 154 and Vol. 155 (Wu and Grossman, and Wu, eds., respectively), Mayer and Walker, eds. (1987), Immunochemical Methods in Cell and Molecular Biology (Academic Press, London), Scopes, (1987), Protein Purification: Principles and Practice, Second Edition (Springer-Verlag, N.Y.), and Handbook of Experimental Immunology, Volumes I-IV (D. M. Weir and C. C. Blackwell eds 1986). For example, bacterial cells can be transformed with an expression vector which comprises the DNA template from which the siRNA is to be derived.

If synthesized outside the cell, the siRNA may be purified prior to introduction into the cell. Purification may be by extraction with a solvent (such as phenol/chloroform) or resin, precipitation (for example in ethanol), electrophoresis, chromatography, or a combination thereof. However, purification may result in loss of siRNA and may therefore be minimal or not carried out at all. The siRNA may be dried for storage or dissolved in an aqueous solution, which may contain buffers or salts to promote annealing, and/or stabilization of the RNA strands.

The double-stranded structure may be formed by a single self-complementary RNA strand or two separate complementary RNA strands.

It is known that mammalian cells can respond to extracellular siRNA and therefore may have a transport mechanism for dsRNA (Asher et al. (1969) Nature 223 715-717). Thus, siRNA may be administered extracellularly into a cavity, interstitial space, into the circulation of a mammal, or introduced orally. Methods for oral introduction include direct mixing of the RNA with food of the mammal, as well as engineered approaches in which a species that is used as food is engineered to express the RNA, then fed to the mammal to be affected. For example, food bacteria, such as Lactococcus lactis, may be transformed to produce the dsRNA (see WO93/17117, WO97/14806). Vascular or extravascular circulation, the blood or lymph systems and the cerebrospinal fluid are sites where the RNA may be injected.

RNA may be introduced into the cell intracellularly. Physical methods of introducing nucleic acids may also be used in this respect. siRNA may be administered using the microinjection techniques described in Zernicka-Goetz et al. (1997) Development 124, 1133-1137 and Wianny et al. (1998) Chromosoma 107, 430-439.

Other physical methods of introducing nucleic acids intracellularly include bombardment by particles covered by the siRNA, for example gene gun technology in which the siRNA is immobilized on gold particles and fired directly at the site of wounding. Thus, the invention provides the use of an siRNA in a gene gun for inhibiting the expression of a target gene. Further, there is provided a composition suitable for gene gun therapy comprising an siRNA and gold particles. An alternative physical method includes electroporation of cell membranes in the presence of the siRNA. This method permits RNAi on a large scale. Other methods known in the art for introducing nucleic acids to cells may be used, such as lipid-mediated carrier transport, chemical-mediated transport, such as calcium phosphate, and the like. siRNA may be introduced along with components that perform one or more of the following activities: enhance RNA uptake by the cell, promote annealing of the duplex strands, stabilize the annealed strands, or otherwise increase inhibition of the target gene.

Any known gene therapy technique can be used to administer the RNA. A viral construct packaged into a viral particle would accomplish both efficient introduction of an expression construct into the cell and transcription of siRNA encoded by the expression construct. Thus, siRNA can also be produced inside a cell. Vectors, e.g., expression vectors that comprise a nucleic acid encoding one or the two strands of an siRNA molecule may be used for that purpose. The nucleic acid may further comprise an antisense sequence that is essentially complementary to the sense target sequence. The nucleic acid may further comprise a spacer sequence between the sense and the antisense target sequence. The nucleic acid may further comprise a promoter for directing expression of the sense and antisense sequences in a cell, e.g., an RNA Polymerase II or III promoter and a transcriptional termination signal. The sequences may be operably linked.

In one embodiment a nucleic acid comprises an RNA coding region (e.g., sense or antisense target sequence) operably linked to an RNA polymerase III promoter. The RNA coding region can be immediately followed by a pol III terminator sequence, which directs termination of RNA synthesis by pol III. The pol III terminator sequences generally have 4 or more consecutive thymidine (“T”) residues. In a preferred embodiment, a cluster of 5 consecutive T residues is used as the terminator by which pol III transcription is stopped at the second or third T of the DNA template, and thus only 2 to 3 uridine (“U”) residues are added to the 3′ end of the coding sequence. A variety of pol III promoters can be used with the invention, including for example, the promoter fragments derived from H1 RNA genes or U6 snRNA genes of human or mouse origin or from any other species. In addition, pol III promoters can be modified/engineered to incorporate other desirable properties such as the ability to be induced by small chemical molecules, either ubiquitously or in a tissue-specific manner. For example, in one embodiment the promoter may be activated by tetracycline. In another embodiment the promoter may be activated by IPTG (lacI system).

siRNAs can be produced in cells by transforming cells with two nucleic acids, e.g., vectors, each nucleic acid comprising an expressing cassette, each expression cassette comprising a promoter, an RNA coding sequence (one being a sense target sequence and the other being an antisense target sequence) and a termination signal. Alternatively, a single nucleic acid may comprise these two expression cassettes. In yet another embodiment, a nucleic acid encodes a single stranded RNA comprising a sense target sequence linked to a spacer linked to an antisense target sequence. The nucleic acids may be present in a vector, such as an expression vector, e.g.; a eukaryotic expression vector that allows expression of the sense and antisense target sequences in cells into which it is introduced.

Vectors for producing siRNAs are described, e.g., in Paul et al. (2002) Nature Biotechnology 29:505; Xia et al., (2002) Nature Biotechnology 20:1006; Zeng et al. (2002) Mol. Cell. 9:1327; Thijn et al., (2002) Science 296:550; BMC Biotechnol. 2002 Aug. 28; 2(1):15; Lee et al. (2002) Nature Biotechnology 19: 500; McManus et al. (2002) RNA 8:842; Miyagishi et al. (2002) Nature Biotechnology 19:497; Sui et al. (2002) PNAS 99:5515; Yu et al. (2002) PNAS 99:6047; Shi et al. (2003) Trends Genet. 19(1):9; Gaudilliere et al. (2002) J. Biol. Chem. 277(48):46442; US2002/0182223; US 2003/0027783; WO 01/36646 and WO 03/006477. Vectors are also available commercially. For example, the pSilencer is available from Gene Therapy Systems, Inc. and pSUPER RNAi system is available from Oligoengine.

Also provided herein are compositions comprising one or more siRNA or nucleic acid encoding an RNA coding region of an siRNA. Compositions may be pharmaceutical compositions and comprise a pharmaceutically acceptable carrier. Compositions may also be provided in a device for administering the composition in a cell or in a subject. For example a composition may be present in a syringe or on a stent. A composition may also comprise agents facilitating the entry of the siRNA or nucleic acid into a cell.

In general, the oligonucleotides may be synthesized using protocols known in the art, for example, as described in Caruthers et al., Methods in Enzymology (1992) 211:3-19; Thompson et al., International PCT Publication No. WO 99/54459; Wincott et al., Nucl. Acids Res. (1995) 23:2677-2684; Wincott et al., Methods Mol. Bio., (1997) 74:59; Brennan et al., Biotechnol. Bioeng. (1998) 61:33-45; and Brennan, U.S. Pat. No. 6,001,311; each of which is hereby incorporated by reference in its entirety herein. In general, the synthesis of oligonucleotides involves conventional nucleic acid protecting and coupling groups, such as dimethoxytrityl at the 5′-end, and phosphoramidites at the 3′-end. In a non-limiting example, small scale syntheses are conducted on a Expedite 8909 RNA synthesizer sold by Applied Biosystems, Inc. (Weiterstadt, Germany), using ribonucleoside phosphoramidites sold by ChemGenes Corporation (Ashland Technology Center, 200 Horner Avenue, Ashland, Mass. 01721, USA). Alternatively, syntheses can be performed on a 96-well plate synthesizer, such as the instrument produced by Protogens (Palo Alto, Calif., USA), or by methods such as those described in Usman et al., J. Am. Chem. Soc. (1987) 109:7845; Scaringe et al., Nucl. Acids Res. (1990) 18:5433; Wincott et al., Nucl. Acids Res. (1990) 23:2677-2684; and Wincott et al., Methods Mol. Bio. (1997) 74:59, each of which is hereby incorporated by reference in its entirety.

The nucleic acid molecules of the present invention may be synthesized separately and dsRNAs may be formed post-synthetically, for example, by ligation (Moore et al., Science (1992) 256:9923; Draper et al., International PCT publication No. WO 93/23569; Shabarova et al., Nucl. Acids Res. (1991) 19:4247; Bellon et al., Nucleosides & Nucleotides (1997) 16:951; and Bellon et al., Bioconjugate Chem. (1997) 8:204; or by hybridization following synthesis and/or deprotection. The nucleic acid molecules can be purified by gel electrophoresis using conventional methods or can be purified by high pressure liquid chromatography (HPLC; see Wincott et al., supra, the totality of which is hereby incorporated herein by reference) and re-suspended in water.

In another embodiment, the level of a particular mRNA or polypeptide in a cell is reduced by introduction of a ribozyme into the cell or nucleic acid encoding such. Ribozyme molecules designed to catalytically cleave mRNA transcripts can also be introduced into, or expressed, in cells to inhibit expression of gene Y (see, e.g., Sarver et al., 1990, Science 247:1222-1225 and U.S. Pat. No. 5,093,246). One commonly used ribozyme motif is the hammerhead, for which the substrate sequence requirements are minimal. Design of the hammerhead ribozyme is disclosed in Usman et al., Current Opin. Struct. Biol. (1996) 6:527-533. Usman also discusses the therapeutic uses of ribozymes. Ribozymes can also be prepared and used as described in Long et al., FASEB J. (1993) 7:25; Symons, Ann. Rev. Biochem. (1992) 61:641; Perrotta et al., Biochem. (1992) 31:16-17; Ojwang et al., Proc. Natl. Acad. Sci. (USA) (1992) 89:10802-10806; and U.S. Pat. No. 5,254,678. Ribozyme cleavage of HIV-I RNA is described in U.S. Pat. No. 5,144,019; methods of cleaving RNA using ribozymes is described in U.S. Pat. No. 5,116,742; and methods for increasing the specificity of ribozymes are described in U.S. Pat. No. 5,225,337 and Koizumi et al., Nucleic Acid Res. (1989) 17:7059-7071. Preparation and use of ribozyme fragments in a hammerhead structure are also described by Koizumi et al., Nucleic Acids Res. (1989) 17:7059-7071. Preparation and use of ribozyme fragments in a hairpin structure are described by Chowrira and Burke, Nucleic Acids Res. (1992) 20:2835. Ribozymes can also be made by rolling transcription as described in Daubendiek and Kool, Nat. Biotechnol. (1997) 15(3):273-277.

Gene expression can be reduced by targeting deoxyribonucleotide sequences complementary to the regulatory region of the target gene (i.e., the gene promoter and/or enhancers) to form triple helical structures that prevent transcription of the gene in target cells in the body. (See generally, Helene (1991) Anticancer Drug Des., 6(6):569-84; Helene et al. (1992) Ann. N.Y. Acad. Sci., 660:27-36; and Maher (1992) Bioassays 14(12):807-15).

In a further embodiment, RNA aptamers can be introduced into or expressed in a cell. RNA aptamers are specific RNA ligands for proteins, such as for Tat and Rev RNA (Good et al. (1997) Gene Therapy 4: 45-54) that can specifically inhibit their translation.

### 4. Sbn Polypeptides

The S. aureus polypeptides, including SbnA, SbnB, SbnC, SbnD, SbnE, SbnF, SbnG, SbnH, and SbnI (FIGS. 2-10; SEQ ID NOs: 4, 7, 10, 13, 16, 19, 22, 25, and 28) described herein, include naturally purified products, products of chemical synthetic procedures, and products produced by recombinant techniques from a prokaryotic or eukaryotic host cell, including for example, bacterial, yeast, higher plant, insect, and mammalian cells. In certain, embodiments, the polypeptides disclosed herein inhibit the function of Sbn polypeptides.

Polypeptides may also comprise, consist of or consist essentially of any of the amino acid sequences described herein. Yet other polypeptides comprise, consist of or consist essentially of an amino acid sequence that has at least about 70%, 80%, 90%, 95%, 98% or 99% identity or homology with a Sbn polypeptide. For example, polypeptides that differ from a sequence in a naturally occurring Sbn protein in about 1, 2, 3, 4, 5 or more amino acids are also contemplated. The differences may be substitutions, e.g., conservative substitutions, deletions or additions. The differences are preferably in regions that are not significantly conserved among different species. Such regions can be identified by aligning the amino acid sequences of Sbn proteins from various species. These amino acids can be substituted, e.g., with those found in another species. Other amino acids that may be substituted, inserted or deleted at these or other locations can be identified by mutagenesis studies coupled with biological assays.

Other proteins that are encompassed herein are those that comprise modified amino acids. Exemplary proteins are derivative proteins that may be one modified by glycosylation, pegylation, phosphorylation or any similar process that retains at least one biological function of the protein from which it was derived.

Proteins may also comprise one or more non-naturally occurring amino acids. For example, nonclassical amino acids or chemical amino acid analogs can be introduced as a substitution or addition into proteins. Non-classical amino acids include, but are not limited to, the D-isomers of the common amino acids, 2,4-diaminobutyric acid, alpha-amino isobutyric acid, 4-aminobutyric acid, Abu, 2-amino butyric acid, gamma-Abu, epsilon-Mix, 6-amino hexanoic acid, Aib, 2-amino isobutyric acid, 3-amino propionic acid, ornithine, norleucine, norvaline, hydroxyproline, sarcosine, citrulline, homocitrulline, cysteic acid, t-butylglycine, t-butylalanine, phenylglycine, cyclohexylalanine, beta-alanine, fluoro-amino acids, designer amino acids such as beta-methyl amino acids, Calpha-methyl amino acids, Nalpha-methyl amino acids, and amino acid analogs in general. Furthermore, the amino acid can be D (dextrorotary) or L (levorotary).

In certain embodiments, a Sbn polypeptide described herein may be a fusion protein containing a domain which increases its solubility and/or facilitates its purification, identification, detection, and/or structural characterization. Exemplary domains, include, for example, glutathione S-transferase (GST), protein A, protein G, calmodulin-binding peptide, thioredoxin, maltose binding protein, HA, myc, poly arginine, poly His, poly His-Asp or FLAG fusion proteins and tags. Additional exemplary domains include domains that alter protein localization in vivo, such as signal peptides, type III secretion system-targeting peptides, transcytosis domains, nuclear localization signals, etc. In various embodiments, a polypeptide of the invention may comprise one or more heterologous fusions. Polypeptides may contain multiple copies of the same fusion domain or may contain fusions to two or more different domains. The fusions may occur at the N-terminus of the polypeptide, at the C-terminus of the polypeptide, or at both the N- and C-terminus of the polypeptide. It is also within the scope of the invention to include linker sequences between a polypeptide of the invention and the fusion domain in order to facilitate construction of the fusion protein or to optimize protein expression or structural constraints of the fusion protein. In another embodiment, the polypeptide may be constructed so as to contain protease cleavage sites between the fusion polypeptide and polypeptide of the invention in order to remove the tag after protein expression or thereafter. Examples of suitable endoproteases, include, for example, Factor Xa and TEV proteases. A protein may also be fused to a signal sequence. For example, when prepared recombinantly, a nucleic acid encoding the peptide may be linked at its 5′ end to a signal sequence, such that the protein is secreted from the cell.

The S. aureus polypeptides can be recovered and purified from recombinant cell cultures by well-known methods including ammonium sulfate or ethanol precipitation, acid extraction, anion or cation exchange chromatography, phosphocellulose chromatography, hydrophobic interaction chromatography, affinity chromatography, hydroxylapatite chromatography, lectin chromatography and high performance liquid chromatography (“HPLC”) is employed for purification. Proteins may be used as a substantially pure preparation, e.g., wherein at least about 90% of the protein in the preparation are the desired protein. Compositions comprising at least about 50%, 60%, 70%, or 80% of the desired protein may also be used.

Proteins may be denatured or non-denatured and may be aggregated or non-aggregated as a result thereof. Proteins can be denatured according to methods known in the art.

In certain embodiments, polypeptides of the invention may be synthesized chemically, ribosomally in a cell free system, or ribosomally within a cell. Chemical synthesis of polypeptides of the invention may be carried out using a variety of art recognized methods, including stepwise solid phase synthesis, semi-synthesis through the conformationally-assisted re-ligation of peptide fragments, enzymatic ligation of cloned or synthetic peptide segments, and chemical ligation. Native chemical ligation employs a chemoselective reaction of two unprotected peptide segments to produce a transient thioester-linked intermediate. The transient thioester-linked intermediate then spontaneously undergoes a rearrangement to provide the full length ligation product having a native peptide bond at the ligation site. Full length ligation products are chemically identical to proteins produced by cell free synthesis. Full length ligation products may be refolded and/or oxidized, as allowed, to form native disulfide-containing protein molecules. (see e.g., U.S. Pat. Nos. 6,184,344 and 6,174,530; and Muir et al., Curr. Opin. Biotech. (1993): vol. 4, p 420; Miller et al., Science (1989): vol. 246, p 1149; Wlodawer et al., Science (1989): vol. 245, p 616; Huang et al., Biochemistry (1991): vol. 30, p 7402; Schnolzer, et al., Int. J. Pept. Prot. Res. (1992): vol. 40, p 180-193; Rajarathnam et al., Science (1994): vol. 264, p 90; R. E. Offord, “Chemical Approaches to Protein Engineering”, in Protein Design and the Development of New therapeutics and Vaccines, J. B. Hook, G. Poste, Eds., (Plenum Press, New York, 1990) pp. 253-282; Wallace et al., J. Biol. Chem. (1992): vol. 267, p 3852; Abrahmsen et al., Biochemistry (1991): vol. 30, p 4151; Chang, et al., Proc. Natl. Acad. Sci. USA (1994) 91: 12544-12548; Schnlzer et al., Science (1992): vol., 3256, p 221; and Akaji et al., Chem. Pharm. Bull. (Tokyo) (1985) 33: 184).

In certain embodiments, it may be advantageous to provide naturally-occurring or experimentally-derived homologs of a polypeptide of the invention. Such homologs may function in a limited capacity as a modulator to promote or inhibit a subset of the biological activities of the naturally-occurring form of the polypeptide. Thus, specific biological effects may be elicited by treatment with a homolog of limited function, and with fewer side effects relative to treatment with agonists or antagonists which are directed to all of the biological activities of a polypeptide of the invention. For instance, antagonistic homologs may be generated which interfere with the ability of the wild-type polypeptide of the invention to associate with certain proteins, but which do not substantially interfere with the formation of complexes between the native polypeptide and other cellular proteins.

Polypeptides may be derived from the full-length polypeptides of the invention. Isolated peptidyl portions of those polypeptides may be obtained by screening polypeptides recombinantly produced from the corresponding fragment of the nucleic acid encoding such polypeptides. In addition, fragments may be chemically synthesized using techniques known in the art such as conventional Merrifield solid phase f-Moc or t-Boc chemistry. For example, proteins may be arbitrarily divided into fragments of desired length with no overlap of the fragments, or may be divided into overlapping fragments of a desired length. The fragments may be produced (recombinantly or by chemical synthesis) and tested to identify those peptidyl fragments having a desired property, for example, the capability of functioning as a modulator of the polypeptides of the invention. In an illustrative embodiment, peptidyl portions of a protein of the invention may be tested for binding activity, as well as inhibitory ability, by expression as, for example, thioredoxin fusion proteins, each of which contains a discrete fragment of a protein of the invention (see, for example, U.S. Pat. Nos. 5,270,181 and 5,292,646; and PCT publication WO94/02502).

In another embodiment, truncated polypeptides may be prepared. Truncated polypeptides have from 1 to 20 or more amino acid residues removed from either or both the N- and C-termini. Such truncated polypeptides may prove more amenable to expression, purification or characterization than the full-length polypeptide. For example, truncated polypeptides may prove more amenable than the full-length polypeptide to crystallization, to yielding high quality diffracting crystals or to yielding an HSQC spectrum with high intensity peaks and minimally overlapping peaks. In addition, the use of truncated polypeptides may also identify stable and active domains of the full-length polypeptide that may be more amenable to characterization.

It is also possible to modify the structure of the polypeptides of the invention for such purposes as enhancing therapeutic or prophylactic efficacy, or stability (e.g., ex vivo shelf life, resistance to proteolytic degradation in vivo, etc.). Such modified polypeptides, when designed to retain at least one activity of the naturally-occurring form of the protein, are considered “functional equivalents” of the polypeptides described in more detail herein. Such modified polypeptides may be produced, for instance, by amino acid substitution, deletion, or addition, which substitutions may consist in whole or part by conservative amino acid substitutions.

For instance, it is reasonable to expect that an isolated conservative amino acid substitution, such as replacement of a leucine with an isoleucine or valine, an aspartate with a glutamate, a threonine with a serine, will not have a major affect on the biological activity of the resulting molecule. Whether a change in the amino acid sequence of a polypeptide results in a functional homolog may be readily determined by assessing the ability of the variant polypeptide to produce a response similar to that of the wild-type protein. Polypeptides in which more than one replacement has taken place may readily be tested in the same manner.

Methods of generating sets of combinatorial mutants of polypeptides of the invention are provided, as well as truncation mutants, and is especially useful for identifying potential variant sequences (e.g., homologs). The purpose of screening such combinatorial libraries is to generate, for example, homologs which may modulate the activity of a polypeptide of the invention, or alternatively, which possess novel activities altogether. Combinatorially-derived homologs may be generated which have a selective potency relative to a naturally-occurring protein. Such homologs may be used in the development of therapeutics.

Likewise, mutagenesis may give rise to homologs which have intracellular half-lives dramatically different than the corresponding wild-type protein. For example, the altered protein may be rendered either more stable or less stable to proteolytic degradation or other cellular process which result in destruction of; or otherwise inactivation of the protein. Such homologs, and the genes which encode them, may be utilized to alter protein expression by modulating the half-life of the protein. As above, such proteins may be used for the development of therapeutics or treatment.

In similar fashion, protein homologs may be generated by the present combinatorial approach to act as antagonists, in that they are able to interfere with the activity of the corresponding wild-type protein.

In a representative embodiment of this method, the amino acid sequences for a population of protein homologs are aligned, preferably to promote the highest homology possible. Such a population of variants may include, for example, homologs from one or more species, or homologs from the same species but which differ due to mutation. Amino acids which appear at each position of the aligned sequences are selected to create a degenerate set of combinatorial sequences. In certain embodiments, the combinatorial library is produced by way of a degenerate library of genes encoding a library of polypeptides which each include at least a portion of potential protein sequences. For instance, a mixture of synthetic oligonucleotides may be enzymatically ligated into gene sequences such that the degenerate set of potential nucleotide sequences are expressible as individual polypeptides, or alternatively, as a set of larger fusion proteins (e.g. for phage display).

There are many ways by which the library of potential homologs may be generated from a degenerate oligonucleotide sequence. Chemical synthesis of a degenerate gene sequence may be carried out in an automatic DNA synthesizer, and the synthetic genes may then be ligated into an appropriate vector for expression. One purpose of a degenerate set of genes is to provide, in one mixture, all of the sequences encoding the desired set of potential protein sequences. The synthesis of degenerate oligonucleotides is well known in the art (see for example, Narang (1983) Tetrahedron 39:3; Itakura et al. (1981) Recombinant DNA, Proc. 3rd Cleveland Sympos. Macromolecules, ed. A G Walton, Amsterdam: Elsevier pp. 273-289; Itakura et al. (1984) Annu. Rev. Biochem. 53:323; Itakura et al. (1984) Science 198:1056; Ike et al., (1983) Nucleic Acid Res. 11:477). Such techniques have been employed in the directed evolution of other proteins (see, for example, Scott et al. (1990) Science 249:386-390; Roberts et al., (1992) PNAS USA 89:2429-2433; Devlin et al. (1990) Science 249: 404-406; Cwirla et al. (1990) PNAS USA 87: 6378-6382; as well as U.S. Pat. Nos. 5,223,409, 5,198,346, and 5,096,815).

Alternatively, other forms of mutagenesis may be utilized to generate a combinatorial library. For example, protein homologs (both agonist and antagonist forms) may be generated and isolated from a library by screening using, for example, alanine scanning mutagenesis and the like (Ruf et al. (1994) Biochemistry 33:1565-1572; Wang et al. (1994) J. Biol. Chem. 269:3095-3099; Balint et al., (1993) Gene 137:109-118; Grodberg et al. (1993) Eur. J. Biochem. 218:597-601; Nagashima et al. (1993) J. Biol. Chem. 268:2888-2892; Lowman et al. (1991) Biochemistry 30:10832-10838; and Cunningham et al., (1989) Science 244:1081-1085), by linker scanning mutagenesis (Gustin et al. (1993) Virology 193:653-660; Brown et al. (1992) Mol. Cell. Biol. 12:2644-2652; McKnight et al. (1982) Science 232:316); by saturation mutagenesis (Meyers et al. (1986) Science 232:613); by PCR mutagenesis (Leung et al. (1989) Method Cell Mol Biol 1:11-19); or by random mutagenesis (Miller et al. (1992) A Short Course in Bacterial Genetics, CSHL Press, Cold Spring Harbor, N.Y.; and Greener et al. (1994) Strategies in Mol Biol 7:32-34). Linker scanning mutagenesis, particularly in a combinatorial setting, is an attractive method for identifying truncated forms of proteins that are bioactive.

A wide range of techniques are known in the art for screening gene products of combinatorial libraries made by point mutations and truncations, and for screening cDNA libraries for gene products having a certain property. Such techniques will be generally adaptable for rapid screening of the gene libraries generated by the combinatorial mutagenesis of protein homologs. The most widely used techniques for screening large gene libraries typically comprises cloning the gene library into replicable expression vectors, transforming appropriate cells with the resulting library of vectors, and expressing the combinatorial genes under conditions in which detection of a desired activity facilitates relatively easy isolation of the vector encoding the gene whose product was detected.

In an illustrative embodiment of a screening assay, candidate combinatorial gene products are displayed on the surface of a cell and the ability of particular cells or viral particles to bind to the combinatorial gene product is detected in a “panning assay”. For instance, the gene library may be cloned into the gene for a surface membrane protein of a bacterial cell (Ladner et al., WO 88/06630; Fuchs et al., (1991) Bio/Technology 9:1370-1371; and Goward et al., (1992) TIBS 18:136-140), and the resulting fusion protein detected by panning, e.g. using a fluorescently labeled molecule which binds the cell surface protein, e.g. FITC-substrate, to score for potentially functional homologs. Cells may be visually inspected and separated under a fluorescence microscope, or, when the morphology of the cell permits, separated by a fluorescence-activated cell sorter. This method may be used to identify substrates or other polypeptides that can interact with a polypeptide of the invention.

In similar fashion, the gene library may be expressed as a fusion protein on the surface of a viral particle. For instance, in the filamentous phage system, foreign peptide sequences may be expressed on the surface of infectious phage, thereby conferring two benefits. First, because these phage may be applied to affinity matrices at very high concentrations, a large number of phage may be screened at one time. Second, because each infectious phage displays the combinatorial gene product on its surface; if a particular phage is recovered from an affinity matrix in low yield, the phage may be amplified by another round of infection. The group of almost identical E. coli filamentous phages M13, fd, and f1 are most often used in phage display libraries, as either of the phage gIII or gVIII coat proteins may be used to generate fusion proteins without disrupting the ultimate packaging of the viral particle (Ladner et al., PCT publication WO 90/02909; Garrard et al., PCT publication WO 92/09690; Marks et al., (1992) J. Biol. Chem. 267:16007-16010; Griffiths et al., (1993) EMBO J. 12:725-734; Clackson et al., (1991) Nature 352:624-628; and Barbas et al., (1992) PNAS USA 89:4457-4461). Other phage coat proteins may be used as appropriate.

The polypeptides disclosed herein may be reduced to generate mimetics, e.g. peptide or non-peptide agents, which are able to mimic binding of the authentic protein to another cellular partner. Such mutagenic techniques as described above, as well as the thioredoxin system, are also particularly useful for mapping the determinants of a protein which participates in a protein-protein interaction with another protein. To illustrate, the critical residues of a protein which are involved in molecular recognition of a substrate protein may be determined and used to generate peptidomimetics that may bind to the substrate protein. The peptidomimetic may then be used as an inhibitor of the wild-type protein by binding to the substrate and covering up the critical residues needed for interaction with the wild-type protein, thereby preventing interaction of the protein and the substrate. By employing, for example, scanning mutagenesis to map the amino acid residues of a protein which are involved in binding a substrate polypeptide, peptidomimetic compounds may be generated which mimic those residues in binding to the substrate.

For instance, derivatives of the Sbn proteins described herein may be chemically modified peptides and peptidomimetics. Peptidomimetics are compounds based on, or derived from, peptides and proteins. Peptidomimetics can be obtained by structural modification of known peptide sequences using unnatural amino acids, conformational restraints, isosteric replacement, and the like. The subject peptidomimetics constitute the continum of structural space between peptides and non-peptide synthetic structures; peptidomimetics may be useful, therefore, in delineating pharmacophores and in helping to translate peptides into nonpeptide compounds with the activity of the parent peptides.

Moreover, mimetopes of the subject peptides can be provided. Such peptidomimetics can have such attributes as being non-hydrolyzable (e.g., increased stability against proteases or other physiological conditions which degrade the corresponding peptide), increased specificity and/or potency for stimulating cell differentiation. For illustrative purposes, non-hydrolyzable peptide analogs of such residues may be generated using benzodiazepine (e.g., see Freidinger et al., in Peptides: Chemistry and Biology, G. R. Marshall ed., ESCOM Publisher: Leiden, Netherlands, 1988), azepine (e.g., see Huffman et al., in Peptides: Chemistry and Biology, G. R. Marshall ed., ESCOM Publisher: Leiden, Netherlands, 1988), substituted gamma lactam rings (Garvey et al., in Peptides: Chemistry and Biology, G. R. Marshall ed., ESCOM Publisher: Leiden, Netherlands, 1988), keto-methylene pseudopeptides (Ewenson et al., (1986) J. Med. Chem. 29:295; and Ewenson et al., in Peptides: Structure and Function (Proceedings of the 9th American Peptide Symposium) Pierce Chemical Co. Rockland, Ill., 1985), β-turn dipeptide cores (Nagai et al., (1985) Tetrahedron Lett 26:647; and Sato et al. (1986) J Chem Soc Perkin Trans 1:1231), and β-aminoalcohols (Gordon et al. (1985) Biochem Biophys Res Commun 126:419; and Dann et al. (1986) Biochem Biophys Res Commun 134:71).

In addition to a variety of sidechain replacements which can be carried out to generate peptidomimetics, the description specifically contemplates the use of conformationally restrained mimics of peptide secondary structure. Numerous surrogates have been developed for the amide bond of peptides. Frequently exploited surrogates for the amide bond include the following groups (i) trans-olefins, fluoroalkene, (iii) methyleneamino, (iv) phosphonamides, and (v) sulfonamides.

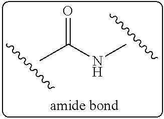

### Examples of Surrogates:

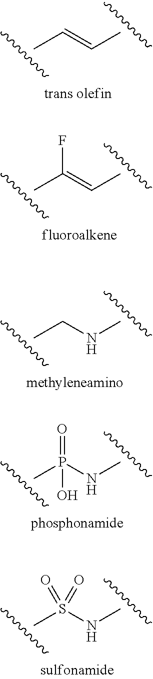

Additionally, peptidomimetics based on more substantial modifications of the backbone of a peptide can be used. Peptidomimetics which fall in this category include (i) retro-inverso analogs, and (ii) N-alkyl glycine analogs (so-called peptoids).

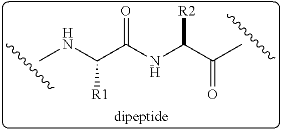

### Examples of Analogs:

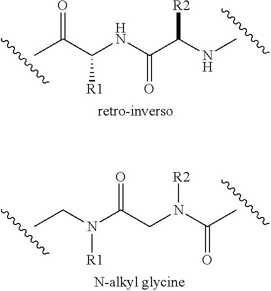

Furthermore, the methods of combinatorial chemistry are being brought to bear, on the development of new peptidomimetics. For example, one embodiment of a so-called “peptide morphing” strategy focuses on the random generation of a library of peptide analogs that comprise a wide range of peptide bond substitutes.

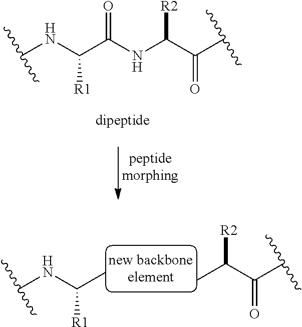

In an exemplary embodiment, the peptidomimetic can be derived as a retro-inverso analog of the peptide. Such retro-inverso analogs can be made according to the methods known in the art, such as that described by the Sisto et al. U.S. Pat. No. 4,522,752. A retro-inverso analog can be generated as described, e.g., in WO 00/01720. It will be understood that a mixed peptide, e.g. including some normal peptide linkages, may be generated. As a general guide, sites which are most susceptible to proteolysis are typically altered, with less susceptible amide linkages being optional for mimetic switching. The final product, or intermediates thereof, can be purified by HPLC.

Peptides may comprise at least one amino acid or every amino acid that is a D stereoisomer. Other peptides may comprise at least one amino acid that is reversed. The amino acid that is reversed may be a D stereoisomer. Every amino acid of a peptide may be reversed and/or every amino acid may be a D stereoisomer.

In another illustrative embodiment, a peptidomimetic can be derived as a retro-enantio analog of a peptide. Retro-enantio analogs such as this can be synthesized with commercially available D-amino acids (or analogs thereof) and standard solid- or solution-phase peptide-synthesis techniques, as described, e.g., in WO 00/01720. The final product may be purified by HPLC to yield the pure retro-enantio analog.

In still another illustrative embodiment, trans-olefin derivatives can be made for the subject peptide. Trans-olefin analogs can be synthesized according to the method of Y. K. Shue et al. (1987) Tetrahedron Letters 28:3225 and as described in WO 00/01720. It is further possible to couple pseudodipeptides synthesized by the above method to other pseudodipeptides, to make peptide analogs with several olefinic functionalities in place of amide functionalities.

Still another class of peptidomimetic derivatives include the phosphonate derivatives. The synthesis of such phosphonate derivatives can be adapted from known synthesis schemes. See, for example, Loots et al. in Peptides: Chemistry and Biology, (Escom Science Publishers, Leiden, 1988, p. 118); Petrillo et al. in Peptides: Structure and Function (Proceedings of the 9th American Peptide Symposium, Pierce Chemical Co. Rockland, Ill., 1985).

Many other peptidomimetic structures are known in the art and can be readily adapted for use in the subject peptidomimetics. To illustrate, a peptidomimetic may incorporate the 1-azabicyclo[4.3.0]nonane surrogate (see Kim et al. (1997) J. Org. Chem. 62:2847), or an N-acyl piperazic acid (see Xi et al. (1998) J. Am. Chem. Soc. 120:80), or a 2-substituted piperazine moiety as a constrained amino acid analogue (see Williams et al. (1996) J. Med. Chem. 39:1345-1348). In still other embodiments, certain amino acid residues can be replaced with aryl and bi-aryl moieties, e.g., monocyclic or bicyclic aromatic or heteroaromatic nucleus, or a biaromatic, aromatic-heteroaromatic, or biheteroaromatic nucleus.

The subject peptidomimetics can be optimized by, e.g., combinatorial synthesis techniques combined with high throughput screening.

Moreover, other examples of mimetopes include, but are not limited to, protein-based compounds, carbohydrate-based compounds; lipid-based compounds, nucleic acid-based compounds, natural organic compounds, synthetically derived organic compounds, anti-idiotypic antibodies and/or catalytic antibodies, or fragments thereof. A mimetope can be obtained by, for example, screening libraries of natural and synthetic compounds for compounds capable of inhibiting cell survival and/or tumor growth. A mimetope can also be obtained, for example, from libraries of natural and synthetic compounds, in particular, chemical or combinatorial libraries (i.e., libraries of compounds that differ in sequence or size but that have the same building blocks). A mimetope can also be obtained by, for example, rational drug design. In a rational drug design procedure, the three-dimensional structure of a compound of the present invention can be analyzed by, for example, nuclear magnetic resonance (NMR) or x-ray crystallography. The three-dimensional structure can then be used to predict structures of potential mimetopes by, for example, computer modelling. The predicted mimetope structures can then be produced by, for example, chemical synthesis, recombinant DNA technology, or by isolating a mimetope from a natural source (e.g., plants, animals, bacteria and fungi).

“Peptides, variants and derivatives thereof” or “peptides and analogs thereof” are included in “peptide therapeutics” and is intended to include any of the peptides or modified forms thereof, e.g., peptidomimetics, described herein. Preferred peptide therapeutics decrease cell survival or increase apoptosis. For example, they may decrease cell survival or increase apoptosis by a factor of at least about 2 fold, 5 fold, 10 fold, 30 fold or 100 fold, as determined, e.g., in an assay described herein.

The activity of a Sbn protein, fragment, or variant thereof may be assayed using an appropriate substrate or binding partner or other reagent suitable to test for the suspected activity as described below.

In another embodiment, the activity of a polypeptide may be determined by assaying for the level of expression of RNA and/or protein molecules. Transcription levels may be determined, for example, using Northern blots, hybridization to an oligonucleotide array or by assaying for the level of a resulting protein product. Translation levels may be determined, for example, using Western blotting or by identifying a detectable signal produced by a protein product (e.g., fluorescence, luminescence, enzymatic activity, etc.). Depending on the particular situation, it may be desirable to detect the level of transcription and/or translation of a single gene or of multiple genes.

Alternatively, it may be desirable to measure the overall rate of DNA replication, transcription and/or translation in a cell. In general this may be accomplished by growing the cell in the presence of a detectable metabolite which is incorporated into the resultant DNA, RNA, or protein product. For example, the rate of DNA synthesis may be determined by growing cells in the presence of BrdU which is incorporated into the newly synthesized DNA. The amount of BrdU may then be determined histochemically using an anti-BrdU antibody.

In other embodiments, polypeptides of the invention may be immobilized onto a solid surface, including, microtiter plates, slides, beads, films, etc. The polypeptides of the invention may be immobilized onto a “chip” as part of an array. An array, having a plurality of addresses, may comprise one or more polypeptides of the invention in one or more of those addresses. In one embodiment, the chip comprises one or more polypeptides of the invention as part of an array of polypeptide sequences.

In other embodiments, polypeptides of the invention may be immobilized onto a solid surface, including, plates, microtiter plates, slides, beads, particles, spheres, films, strands, precipitates, gels, sheets, tubing, containers, capillaries, pads, slices, etc. The polypeptides of the invention may be immobilized onto a “chip” as part of an array. An array, having a plurality of addresses, may comprise one or more polypeptides of the invention in one or more of those addresses. In one embodiment, the chip comprises one or more polypeptides of the invention as part of an array.

### 5. Antibodies and Uses Thereof

To produce antibodies against the Sbn polypeptides described herein, host animals may be injected with Sbn polypeptides or with Sbn peptides. Hosts may be injected with peptides of different lengths encompassing a desired target sequence. For example, peptide antigens that are at least 5, 10, 15, 20, 25, 30, 35, 40, 45, 50, 55, 60, 65, 70, 75, 80, 85, 90, 95, 100, 105, 110, 115, 120, 125, 130, 135, 140, 145 or 150 amino acids may be used. Alternatively, if a portion of a protein defines an epitope, but is too short to be antigenic, it may be conjugated to a carrier molecule in order to produce antibodies. Some suitable carrier molecules include keyhole limpet hemocyanin, Ig sequences, TrpE, and human or bovine serum albumen. Conjugation may be carried out by methods known in the art. One such method is to combine a cysteine residue of the fragments with a cysteine residue on the carrier molecule.

In addition, antibodies to three-dimensional epitopes, i.e., non-linear epitopes, may also be prepared, based on, e.g., crystallographic data of proteins. Antibodies obtained from that injection may be screened against the short antigens of proteins described herein. Antibodies prepared against a Sbn peptide may be tested for activity against that peptide as well as the full length Sbn protein. Antibodies may have affinities of at least about 10⁻⁶M, 10⁻⁷M, 10⁻⁸M, 10⁻⁹M, 10⁻¹⁰M, 10⁻¹¹M or 10⁻¹²M or higher toward the Sbn peptide and/or the full length Sbn protein described herein.

Suitable cells for the DNA sequences and host cells for antibody expression and secretion can be obtained from a number of sources, including the American Type Culture Collection (“Catalogue of Cell Lines and Hybridomas” 5^{th} edition (1985) Rockville, Md., U.S.A.).

Polyclonal and monoclonal antibodies may be produced by methods known in the art. Monoclonal antibodies may be produced by hybridomas prepared using known procedures including the immunological method described by Kohler and Milstein, Nature 1975; 256: 495-7; and Campbell in “Monoclonal Antibody Technology, The Production and Characterization of Rodent and Human Hybridomas” in Burdon et al., Eds. Laboratory Techniques in Biochemistry and Molecular Biology, Volume 13, Elsevier Science Publishers, Amsterdam (1985); as well as by the recombinant DNA method described by Huse et al, Science (1989) 246: 1275-81.

Methods of antibody purification are well known in the art. See, for example, Harlow and Lane (1988) Antibodies: A Laboratory Manual, Cold Spring Harbor Laboratory, N.Y. Purification methods may include salt precipitation (for example, with ammonium sulfate), ion exchange chromatography (for example, on a cationic or anionic exchange column run at neutral pH and eluted with step gradients of increasing ionic strength), gel filtration chromatography (including gel filtration HPLC), and chromatography on affinity resins such as protein A, protein G, hydroxyapatite, and anti-antibody. Antibodies may also be purified on affinity columns according to methods known in the art.

Other embodiments include functional equivalents of antibodies, and include, for example, chimerized, humanized, and single chain antibodies as well as fragments thereof. Methods of producing functional equivalents are disclosed in PCT Application WO 93/21319; European Patent Application No. 239,400; PCT Application WO 89/09622; European Patent Application 388,745; and European Patent Application EP 332,424.

Functional equivalents include polypeptides with amino acid sequences substantially the same as the amino acid sequence of the variable or hypervariable regions of the antibodies of the invention. “Substantially the same” amino acid sequence is defined herein as a sequence with at least 70%, preferably at least about 80%, and more preferably at least 90% homology to another amino acid sequence as determined by the FASTA search method in accordance with Pearson and Lipman, (1988) Proc Natl Acd Sci USA 85: 2444-8.

Chimerized antibodies may have constant regions derived substantially or exclusively from human antibody constant regions and variable regions derived substantially or exclusively from the sequence of the variable region from a mammal other than a human. Humanized antibodies may have constant regions and variable regions other than the complement determining regions (CDRs) derived substantially or exclusively from the corresponding human antibody regions and CDRs derived substantially or exclusively from a mammal other than a human.

Suitable mammals other than a human may include any mammal from which monoclonal antibodies may be made. Suitable examples of mammals other than a human may include, for example, a rabbit, rat, mouse, horse, goat, or primate.

Antibodies to Sbn proteins as described herein may be prepared as described above. In a further embodiment, the antibodies to the Sbn proteins described herein (whole antibodies or antibody fragments) may be conjugated to a biocompatible material, such as polyethylene glycol molecules (PEG) according to methods well known to persons of skill in the art to increase the antibody's half-life. See for example, U.S. Pat. No. 6,468,532. Functionalized PEG polymers are available, for example, from Nektar Therapeutics. Commercially available PEG derivatives include, but are not limited to, amino-PEG, PEG amino acid esters, PEG-hydrazide, PEG-thiol, PEG-succinate, carboxymethylated PEG, PEG-propionic acid, PEG amino acids, PEG succinimidyl succinate, PEG succinimidyl propionate, succinimidyl ester of carboxymethylated PEG, succinimidyl carbonate of PEG, succinimidyl esters of amino acid PEGs, PEG-oxycarbonylimidazole, PEG-nitrophenyl carbonate, PEG tresylate, PEG-glycidyl ether, PEG-aldehyde, PEG vinylsulfone, PEG-maleimide, PEG-orthopyridyl-disulfide, heterofunctional PEGs, PEG vinyl derivatives, PEG silanes, and PEG phospholides. The reaction conditions for coupling these PEG derivatives will vary depending on the polypeptide, the desired degree of PEGylation, and the PEG derivative utilized. Some factors involved in the choice of PEG derivatives include: the desired point of attachment (such as lysine or cysteine R-groups), hydrolytic stability and reactivity of the derivatives, stability, toxicity and antigenicity of the linkage, suitability for analysis, etc.

### 6. Pharmaceutical Compositions

S. aureus Sbn antibodies, antisense nucleic acids, siRNAs, and other antagonists, may be administered by various means, depending on their intended use, as is well known in the art. For example, if such S. aureus antagonists compositions are to be administered orally, they may be formulated as tablets, capsules, granules, powders or syrups. Alternatively, formulations of the present invention may be administered parenterally as injections (intravenous, intramuscular or subcutaneous), drop infusion preparations or suppositories. For application by the ophthalmic mucous membrane route, compositions of the present invention may be formulated as eyedrops or eye ointments. These formulations may be prepared by conventional means, and, if desired, the compositions may be mixed with any conventional additive, such as an excipient, a binder, a disintegrating agent, a lubricant, a corrigent, a solubilizing agent, a suspension aid, an emulsifying agent or a coating agent.

In formulations of the subject invention, wetting agents, emulsifiers and lubricants, such as sodium lauryl sulfate and magnesium stearate, as well as coloring agents, release agents, coating agents, sweetening, flavoring and perfuming agents, preservatives and antioxidants may be present in the formulated agents.

Subject compositions may be suitable for oral, nasal, topical (including buccal and sublingual), rectal, vaginal, aerosol and/or parenteral administration. The formulations may conveniently be presented in unit dosage form and may be prepared by any methods well known in the art of pharmacy. The amount of composition that may be combined with a carrier material to produce a single dose vary depending upon the subject being treated, and the particular mode of administration.

Methods of preparing these formulations include the step of bringing into association compositions of the present invention with the carrier and, optionally, one or more accessory ingredients. In general, the formulations are prepared by uniformly and intimately bringing into association agents with liquid carriers, or finely divided solid carriers, or both, and then, if necessary, shaping the product.

Formulations suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia), each containing a predetermined amount of a subject composition thereof as an active ingredient. Compositions of the present invention may also be administered as a bolus, electuary, or paste.

In solid dosage forms for oral administration (capsules, tablets, pills, dragees, powders, granules and the like), the subject composition is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as; for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, acetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the subject composition moistened with an inert liquid diluent. Tablets, and other solid dosage forms, such as dragees, capsules, pills and granules, may optionally be scored or prepared with coatings and shells, such as enteric coatings and other coatings well known in the pharmaceutical-formulating art.

Liquid dosage forms for oral administration include pharmaceutically acceptable emulsions, microemulsions, solutions, suspensions, syrups and elixirs. In addition to the subject composition, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Suspensions, in addition to the subject composition, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations for rectal or vaginal administration may be presented as a suppository, which may be prepared by mixing a subject composition with one or more suitable non-irritating excipients or carriers comprising, for example, cocoa butter, polyethylene glycol, a suppository wax or a salicylate, and which is solid at room temperature, but liquid at body temperature and, therefore, will melt in the body cavity and release the active agent. Formulations which are suitable for vaginal administration also include pessaries, tampons, creams, gels, pastes, foams or spray formulations containing such carriers as are known in the art to be appropriate.

Dosage forms for transdermal administration of a subject composition includes powders, sprays, ointments, pastes, creams, lotions, gels, solutions, patches and inhalants. The active component may be mixed under sterile conditions with a pharmaceutically acceptable carrier, and with any preservatives, buffers, or propellants which may be required.

The ointments, pastes, creams and gels may contain, in addition to a subject composition, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays may contain, in addition to a subject composition, excipients such as lactose, talc, silicic acid, aluminum hydroxide, calcium silicates and polyamide powder, or mixtures of these substances. Sprays may additionally contain customary propellants, such as chlorofluorohydrocarbons and volatile unsubstituted hydrocarbons, such as butane and propane.

Compositions of the present invention may alternatively be administered by aerosol. This is accomplished by preparing an aqueous aerosol, liposomal preparation or solid particles containing the compound. A non-aqueous (e.g., fluorocarbon propellant) suspension could be used. Sonic nebulizers may be used because they minimize exposing the agent to shear, which may result in degradation of the compounds contained in the subject compositions.

Ordinarily, an aqueous aerosol is made by formulating an aqueous solution or suspension of a subject composition together with conventional pharmaceutically acceptable carriers and stabilizers. The carriers and stabilizers vary with the requirements of the particular subject composition, but typically include non-ionic surfactants (Tweens, Pluronics, or polyethylene glycol), innocuous proteins like serum albumin, sorbitan esters, oleic acid, lecithin, amino acids such as glycine, buffers, salts, sugars or sugar alcohols. Aerosols generally are prepared from isotonic solutions.

Pharmaceutical compositions of this invention suitable for parenteral administration comprise a subject composition in combination with one or more pharmaceutically-acceptable sterile isotonic aqueous or non-aqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and non-aqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity may be maintained, for example, by the use of coating materials, such as lecithin, by the maintenance of the required particle size in the case of dispersions, and by the use of surfactants.

The pharmaceutical compositions described herein may be used to prevent or treat conditions or diseases resulting from S. aureus infections including, but not limited to a furuncle, chronic furunculosis, impetigo, acute osteomyelitis, pneumonia, endocarditis, scalded skin syndrome, toxic shock syndrome, and food poisoning.

### 7. Exemplary Screening Assays for Inhibitors of sbn-Mediated Siderophore Biosynthesis

In general, agents or compounds capable of reducing pathogenic virulence by interfering with staphylobactin biosynthesis can be identified using the instant disclosed assays to screen large libraries of both natural product or synthetic (or semi-synthetic) extracts or chemical libraries. Those skilled in the field of drug discovery and development will understand that the precise source of agents (e.g., test extracts or compounds) is not critical to the screening procedures of the invention. Accordingly, virtually any number of chemical extracts or compounds can be screened using the methods described herein. Examples of such agents, extracts, or compounds include, but are not limited to, plant-, fungal-, prokaryotic- or animal-based extracts, fermentation broths, and synthetic compounds, as well as modification of existing compounds. Numerous methods are also available for generating random or directed synthesis (e.g., semi-synthesis or total synthesis) of any number of chemical compounds, including, but not limited to, saccharide-, lipid-, peptide-, and nucleic acid-based compounds. Synthetic compound libraries are commercially available from Brandon Associates (Merrimack, N.H.) and Aldrich Chemical (Milwaukee, Wis.). Alternatively, libraries of natural compounds in the form of bacterial, fungal, plant, and animal extracts are commercially available from a number of sources, including Biotics (Sussex, UK), Xenova (Slough, UK), Harbor Branch Oceangraphics Institute (Ft. Pierce, Fla.), and PharmnaMar, U.S.A. (Cambridge, Mass.). In addition, natural and synthetically produced libraries are produced, if desired, according to methods known in the art, for example, by standard extraction and fractionation methods. Furthermore, if desired, any library or compound is readily modified using standard chemical, physical, or biochemical methods.

In addition, those skilled in the art of drug discovery and development readily understand that methods for dereplication (e.g., taxonomic dereplication, biological dereplication, and chemical dereplication, or any combination thereof) or the elimination of replicates or repeats of materials already known for their anti-pathogenic activity should be employed whenever possible.

When a crude extract is found to have an anti-pathogenic or anti-virulence activity, or a binding activity, further fractionation of the positive lead extract is necessary to isolate chemical constituents responsible for the observed effect. Thus, the goal of the extraction, fractionation, and purification process is the careful characterization and identification of a chemical entity within the crude extract having anti-pathogenic activity. Methods of fractionation and purification of such heterogeneous extracts are known in the art. If desired, compounds shown to be useful agents for the treatment of pathogenicity are chemically modified according to methods known in the art.

Potential inhibitors or antagonists of sbn encoded polypeptides or staphylobactin may include organic molecules, peptides, peptide mimetics, polypeptides, and antibodies that bind to a nucleic acid sequence or polypeptide of the invention and thereby inhibit or extinguish its activity. Potential antagonists also include small molecules that bind to and occupy the binding site of the polypeptide thereby preventing binding to cellular binding molecules, such that normal biological activity is prevented. Other potential antagonists include antisense molecules.

### 7.1 Interaction Assays

Purified and recombinant SbnA, SbnB, SbnC, SbnC, SbnD, SbnB, SbnF, SbnG, SbnH and SbnI polypeptides may be used to develop assays to screen for agents that bind to an Sbn gene product, and disrupt a protein-protein interaction. Potential inhibitors or antagonists of SbnA, SbnB, SbnC, SbnC, SbnD, SbnE, SbnF, SbnG, SbnH or SbnI may include small organic molecules, peptides, polypeptides, peptide mimetics, and antibodies that bind to either SbnA, SbnB, SbnC, SbnC, SbnD, SbnE, SbnF, SbnG, SbnH or SbnI and thereby reduce or extinguish its activity.

In an exemplary binding assay, a reaction mixture may be generated to include at least a biologically active portion of either SbnA, SbnB, SbnC, SbnC, SbnD, SbnE, SbnF, SbnG, SbnH or SbnI, an agent(s) of interest, and an appropriate interacting molecule. The interacting molecule will depend on the Sbn polypeptide to be tested. In a preferred embodiment, the agent of interest is an antibody against a particular Sbn polypeptide. Binding of an antibody to a Sbn polypeptide may inhibit the function of the Sbn polypeptide in the biosynthesis of siderophore. Detection and quantification of an interaction of a particular Sbn polypeptide with an appropriate interacting molecule provides a means for determining an agent's efficacy at inhibiting the interaction. The efficacy of the agent can be assessed by generating dose response curves from data obtained using various concentrations of the test agent. Moreover, a control assay can also be performed to provide a baseline for comparison. In the control assay, the interaction of a particular Sbn polypeptide with an appropriate interacting molecule may be quantitated in the absence of the test agent.

Interaction between a particular Sbn polypeptide and an appropriate interacting molecule may be detected by a variety of techniques. Modulation of the formation of complexes can be quantitated using, for example, detectably labeled proteins such as radiolabeled, fluorescently labeled, or enzymatically labeled polypeptides, by immunoassay, or by chromatographic detection.

The measurement of the interaction of a particular Sbn protein with the appropriate interacting molecule may be observed directly using surface plasmon resonance technology in optical biosensor devices. This method is particularly useful for measuring interactions with larger (>5 kDa) polypeptides and can be adapted to screen for inhibitors of the protein-protein interaction.

Alternatively, it will be desirable to immobilize a particular Sbn polypeptide or the appropriate interacting molecule to facilitate separation of complexes from uncomplexed forms of one or both of the proteins, as well as to accommodate automation of the assay. Binding of a particular Sbn protein to the interacting molecule for example, in the presence and absence of a candidate agent, can be accomplished in any vessel suitable for containing the reactants. Examples include microtitre plates, test tubes, and micro-centrifuge tubes. In one embodiment, a fusion protein can be provided which adds a domain that allows the protein to be bound to a matrix. For example, glutathione-S-transferase/SbnA (GST/SbnA) fusion proteins can be adsorbed onto glutathione sepharose beads (Sigma Chemical, St. Louis, Mo.) or glutathione derivatized microtitre plates, which are then combined with, for example, an ³⁵S-labeled interacting molecule, and the test agent, and the mixture incubated under conditions conducive to complex formation, for example, at physiological conditions for salt and pH, though slightly more stringent conditions may be desired. Following incubation, the beads are washed to remove any unbound label, and the matrix immobilized and radiolabel determined directly (e.g., beads placed in scintillant), or in the supernatant after the complexes are subsequently dissociated. Alternatively, the complexes can be dissociated from the matrix, separated by SDS-PAGE, and the level of interacting molecule found in the bead fraction quantitated from the gel using standard electrophoretic techniques.

Other techniques for immobilizing proteins and other molecules on matrices are also available for use in the subject assay. For instance, either a particular Sbn protein or the appropriate interacting molecule can be immobilized utilizing conjugation of biotin and streptavidin. For instance, biotinylated SbnA, SbnB, SbnC, SbnC, SbnD, SbnE, SbnF, SbnG, SbnH or SbnI can be prepared from biotin-NHS (N-hydroxy-succinimide) using techniques well known in the art (e.g., biotinylation kit, Pierce Chemicals, Rockford, and immobilized in the wells of streptavidin-coated 96 well plates (Pierce Chemical). Alternatively, antibodies reactive with either SbnA, SbnB, SbnC, SbnC, SbnD, SbnE, SbnF, SbnG, SbnH or SbnI, but which do not interfere with the interaction between the polypeptide and the interacting molecule, can be derivatized to the wells of the plate, and SbnA, SbnB, SbnC, SbnC, SbnD, SbnE, SbnF, SbnG, SbnH or SbnI may be trapped in the wells by antibody conjugation. As above, preparations of an interacting molecule and a test compound may be incubated in the polypeptide-presenting wells of the plate, and the amount of complex trapped in the well can be quantitated in the presence or absence of a test agent. Exemplary methods for detecting such complexes, in addition to those described above for the GST-immobilized complexes, include immunodetection of complexes using antibodies reactive with the interacting molecule or enzyme-linked assays which rely on detecting an enzymatic activity associated with the interacting molecule.

For example, an enzyme can be chemically conjugated or provided as a fusion protein with the interacting molecule. To illustrate, the interacting molecule can be chemically cross-linked or genetically fused with horseradish peroxidase, and the amount of polypeptide trapped in the complex can be assessed with a chromogenic substrate of the enzyme, for example, 3,3′-diamino-benzadine tetrahydrochloride or 4-chloro-1-napthol. Likewise, a fusion protein comprising the polypeptide and glutathione-S-transferase can be provided, and complex formation quantitated by detecting the GST activity using 1-chloro-2,4-dinitrobenzene (Habig et al. (1974) J. Biol. Chem. 249:7130).

### 7.2 Biochemical Assays

Purified and recombinant SbnA, SbnB, SbnC, SbnC, SbnD, SbnE, SbnF, SbnG, SbnH and SbnI polypeptides may be used to facilitate the development of assays to screen for agents that inhibit the biosynthetic activity of each gene product comprising the sbn operon. Potential inhibitors or antagonists of SbnA, SbnB, SbnC, SbnC, SbnD, SbnE, SbnF, SbnG, SbnH or SbnI may include small organic molecules, peptides, polypeptides, peptide mimetics, and antibodies that bind to either SbnA, SbnB, SbnC, SbnC, SbnD, SbnE, SbnF, SbnG, SbnH or SbnI and thereby reduce or extinguish its activity.

In an exemplary screening assay, a reaction mixture may be generated to include at least a biologically active portion of either SbnA, SbnB, SbnC, SbnC, SbnD, SbnE, SbnF, SbnG, SbnH or SbnI, a test agent(s) of interest, and a substrate. The appropriate substrate will depend on which Sbn polypeptide is being used in the screening assay. For example, in one exemplary assay, SbnB converts L-ornithine to L-proline and this reaction can be monitored by two methods. One is monitoring the conversion of NAD+ to NADH using a spectrophotometric assay for the reduction of NAD+. The second is using an HPLC-based assay to monitor the conversion of L-ornithine to L-proline. This reaction occurs early in the biosynthesis of staphylobactin. In another assay, SbnA activity is monitored by an HPLC-based assay. SbnA converts O-acetyl-L-serine to L-2,3-diaminopropionic acid. The reaction product is again monitored by HPLC-based methods. The reaction requires the participation of SbnB since the amine group provided by the L-ornithine is used during the conversion of O-acetyl-L-serine to L-2,3-diaminopropionic acid. SbnH activity can also be measured using HPLC. This enzyme likely converts L-ornithine into putrescine.

### 7.3 Expression Assays

In a further embodiment, antagonists of staphylobactin biosynthesis may affect the expression of sbnA, sbnB, sbnC, sbnD, sbnE, sbnF, sbnG, sbnH, and sbnI nucleic acid or protein. In this screen, S. aureus cells may be treated with a compound(s) of interest, and then assayed for the effect of the compound(s) on sbnA, sbnB, sbnC, sbnD, sbnE, sbnF, sbnG, sbnH, and sbnI nucleic acid or protein expression.

For example, total RNA can be isolated from S. aureus cells cultured in the presence or absence of test agents, using any suitable technique such as the single-step guanidinium-thiocyanate-phenol-chloroform method described in Chomczynski et al. (1987) Anal. Biochem. 162:156-159. The expression of sbnA, sbnB, sbnC, sbnD, sbnE, sbnF, sbnG, sbnH or sbnI may then be assayed by any appropriate method such as Northern blot analysis, the polymerase chain reaction (PCR), reverse transcription in combination with the polymerase chain reaction (RT-PCR), and reverse transcription in combination with the ligase chain reaction (RT-LCR).

Northern blot analysis can be performed as described in Harada et al. (1990) Cell 63:303-312. Briefly, total RNA is prepared from S. aureus cells cultured in the presence of a test agent. For the Northern blot, the RNA is denatured in an appropriate buffer (such as glyoxal/dimethyl sulfoxide/sodium phosphate buffer), subjected to agarose gel electrophoresis, and transferred onto a nitrocellulose filter. After the RNAs have been linked to the filter by a UV linker, the filter is prehybridized in a solution containing formamide, SSC, Denhardt's solution, denatured salmon sperm, SDS, and sodium phosphate buffer. A S. aureus sbnA, sbnB, sbnC, sbnD, sbnE, sbnF, sbnG, sbnH or sbnI DNA sequence may be labeled according to any appropriate method (such as the ³²P-multiprimed DNA labeling system (Amersham)) and used as probe. After hybridization overnight, the filter is washed and exposed to x-ray film. Moreover, a control can also be performed to provide a baseline for comparison. In the control, the expression of sbnA, sbnB, sbnC, sbnD, sbnE, sbnF, sbnG, sbnH or sbnI in S. aureus may be quantitated in the absence of the test agent.

Alternatively, the levels of mRNA encoding SbnA, SbnB, SbnC, SbnD, SbnE, SbnF, SbnG, SbnH or SbnI polypeptides may also be assayed; for e.g., using the RT-PCR method described in Makino et al. (1990) Technique 2:295-301. Briefly, this method involves adding total RNA isolated from S. aureus cells cultured in the presence of a test agent, in a reaction mixture containing a RT primer and appropriate buffer. After incubating for primer annealing, the mixture can be supplemented with a RT buffer, dNTPs, DTT, RNase inhibitor and reverse transcriptase. After incubation to achieve reverse transcription of the RNA, the RT products are then subject to PCR using labeled primers. Alternatively, rather than labeling the primers, a labeled dNTP can be included in the PCR reaction mixture. PCR amplification can be performed in a DNA thermal cycler according to conventional techniques. After a suitable number of rounds to achieve amplification, the PCR reaction mixture is electrophoresed on a polyacrylamide gel. After drying the gel, the radioactivity of the appropriate bands may be quantified using an imaging analyzer. RT and PCR reaction ingredients and conditions, reagent and gel concentrations, and labeling methods are well known in the art. Variations on the RT-PCR method will be apparent to the skilled artisan. Other PCR methods that can detect the nucleic acid of the present invention can be found in PCR Primer: A Laboratory Manual (Dieffenbach et al. eds., Cold Spring Harbor Lab Press, 1995). A control can also be performed to provide a baseline for comparison. In the control, the expression of sbnA, sbnB, sbnC, sbnD, sbnE, sbnF, sbnG, sbnH, or sbnI in S. aureus may be quantitated in the absence of the test agent.

Alternatively, the expression of SbnA, SbnB, SbnC, SbnD, SbnE, SbnF, SbnG, SbnH, and SbnI polypeptides may be quantitated following the treatment of S. aureus cells with a test agent using antibody-based methods such as immunoassays. Any suitable immunoassay can be used, including, without limitation, competitive and non-competitive assay systems using techniques such as western blots, radioimmunoassays, ELISA (enzyme-linked immunosorbent assay), “sandwich” immunoassays, immunoprecipitation assays, precipitin reactions, gel diffusion precipitin reactions, immunodiffusion assays, agglutination assays, complement-fixation assays, immunoradiometric assays, fluorescent immunoassays and protein A immunoassays.

For example, SbnA, SbnB, SbnC, SbnD, SbnE, SbnF, SbnG, SbnH or SbnI polypeptides can be detected in a sample obtained from S. aureus cells treated with a test agent, by means of a two-step sandwich assay. In the first step, a capture reagent (e.g., either a SbnA, SbnB, SbnC, SbnD, SbnE, SbnF, SbnG, SbnH or SbnI antibody) is used to capture the specific polypeptide. The capture reagent can optionally be immobilized on a solid phase. In the second step, a directly or indirectly labeled detection reagent is used to detect the captured marker. In one embodiment, the detection reagent is an antibody. The amount of SbnA, SbnB, SbnC, SbnD, SbnE, SbnF, SbnG; SbnH or SbnI polypeptide present in S. aureus cells treated with a test agent can be calculated by reference to the amount present in untreated S. aureus cells.

Suitable enzyme labels include, for example, those from the oxidase group, which catalyze the production of hydrogen peroxide by reacting with substrate. Glucose oxidase is particularly preferred as it has good stability and its substrate (glucose) is readily available. Activity of an oxidase label may be assayed by measuring the concentration of hydrogen peroxide formed by the enzyme-labeled antibody/substrate reaction. Besides enzymes, other suitable labels include radioisotopes, such as iodine (¹²⁵I, ¹²¹I), carbon (¹⁴C), sulphur (³⁵S), tritium (³H).

Examples of suitable fluorescent labels include a fluorescein label, an isothiocyanate label, a rhodamine label, a phycoerythrin label, a phycocyanin label, an allophycocyanin label, an o-phthaldehyde label, and a fluorescamine label.

Examples of suitable enzyme labels include malate dehydrogenase, staphylococcal nuclease, delta-5-steroid isomerase, yeast-alcohol dehydrogenase, alpha-glycerol phosphate dehydrogenase, triose phosphate isomerase, peroxidase; alkaline phosphatase, asparaginase, glucose oxidase, beta-galactosidase, ribonuclease, urease, catalase, glucose-6-phosphate dehydrogenase, glucoamylase, and acetylcholine esterase. Examples of chemiluminescent labels include a luminol label, an isoluminol label, an aromatic acridinium ester label, an imidazole label, an acridinium salt label, an oxalate ester label, a luciferin label, a luciferase label, and an aequorin label.

### EXEMPLIFICATION

The invention, having been generally described, may be more readily understood by reference to the following examples, which are included merely for purposes of illustration of certain aspects and embodiments of the present invention, and are not intended to limit the invention in any way.

### Example 1

### Materials and Methods

### Bacterial Strains, Plasmids and Growth Media

Bacterial strains and plasmids used herein are described in Table 1. E. coli and S. aureus strains were routinely cultured in Luria-Bertani broth (Difco) and tryptic soy broth (Difco), respectively. Iron-restricted bacterial growth was performed in Tris-minimal succinate medium (TMS), the composition of which has been described (Sebulsky et al., (2000) J. Bacteriol. 182:4394-4400). Residual free iron was chelated from TMS medium by the addition of ethylenediamine-di(o-hydroxyphenylacetic acid) (EDDHA) (1 μM unless otherwise stated), or TMS was made iron-replete by the addition of 50 μM FeCl₃. Antibiotics were used at the following concentrations: erythromycin (5 μg/ml), lincomycin (20 μg/ml), neomycin (50 μg/ml), kanamycin (50 μg/ml) and tetracycline (4 μg/ml) for S. aureus selection, and ampicillin (100 μg/ml), tetracycline (10 μg/ml) and erythromycin (300 μg/ml) for E. coli selection. All reagents were made with water purified through a Milli-Q water purification system (Millipore, Mississauga, Ontario, Canada).

### Recombinant DNA Methodology

Plasmid DNA was isolated from E. coli using Qiaprep mini-spin kits (Qiagen). DNA manipulations, including restriction enzyme digestion and DNA ligation, were performed according to standard procedures (Sambrook et al., (1989) Molecular cloning. A laboratory manual, 2nd ed. Cold Spring Harbor Laboratory Press, Cold Spring Harbor). Restriction enzymes were purchased from Life Technologies, MBI Fermentas, New England Biolabs or Roche Diagnostics, and DNA ligations were performed using the Roche Rapid DNA Ligation Kit. PwoI (Roche) was used for all polymerase chain reactions. Oligonucleotides were obtained from Life Technologies and are described in Table 1.

Chromosomal DNA Isolation and Southern blotting

Chromosomal DNA was isolated from various staphylococcal strains using procedures as previously described (Sebulsky et al., (2000) J. Bacteriol. 182:4394-4400). Briefly, cells were lysed at 37° C. using 10 μg of lysostaphin (Sigma) in STE (0.1 M NaCl, 10 mM Tris-HCl, pH 8.0 and 1 mM EDTA, pH 8.0) or, for coagulase-negative staphylococci, lysozyme (1 μg) was added to STE. SDS (0.1%) and proteinase K (0.5 mg) were added to the preparations and incubated 2 h at 55° C. Southern blotting techniques were performed essentially as previously described (Sambrook et al., (1989) Molecular cloning. A laboratory manual, 2nd ed. Cold Spring Harbor Laboratory Press; Cold Spring Harbor), and hybridization was performed with Digoxigenin (DIG) (Roche Diagnostics) labeled probes, prepared and used according to manufacturer's instructions. Light emission was detected by exposing blots to Hyperfilm ECL (Amersham Biosciences).

Construction of a sbnE Mutant

A 3037-bp DNA fragment carrying sbnE was PCR-amplified from the chromosome of S. aureus RN6390 and cloned into pBCSK⁺ (BamHI), generating pSED12. The sbnE coding region was interrupted at a unique NcoI site (end-polished with Klenow enzyme) by the insertion of a kanamycin resistance cassette, derived from plasmid pDG782, to create pSED17. A BamHI fragment containing the disrupted sbnE gene was removed from pSED17 and cloned into the temperature-sensitive S. aureus suicide plasmid, pAUL-A, to generate pSED18. Plasmid pSED18 was introduced into S. aureus RN4220 before being transduced into S. aureus RN6390 using bacteriophage 80α, using methods previously described (Sebulsky et al., (2000) J. Bacteriol. 182:4394-4400). S. aureus RN6390 carrying pSED18 was grown to mid-log phase at 30° C. before the growth temperature was shifted to 42° C. After four hours incubation at 42° C., the culture was plated onto medium containing kanamycin and neomycin and incubated at 42° C. overnight. The sbnE mutant, resistant to kanamycin and neomycin and sensitive to erythromycin and lincomycin, was isolated as a result of allelic exchange between chromosomal sbnE and the insertionally-inactivated copy. The chromosomal insertion of the Km^{r} cassette into sbnE was confirmed by PCR.

Creation of Transcriptional lacZ Fusions and P-Galactosidase Assays

Internal fragments of individual genes were cloned into the multiple cloning site of pMUTIN4 (Vagner et al., (1998) Microbiology. 144:3097-3104), a vector that does not replicate in Gram-positive bacteria. S. aureus RN4220 was then transformed with recombinant pMUTIN4 plasmids and homologous recombination between the cloned DNA sequences and those present on the chromosome resulted in the integration of recombinant plasmids into the chromosome. Chromosomal integrations were confirmed by PCR-amplification of pMUTIN4-specific DNA sequences.

S. aureus strains bearing transcriptional fusions to lacZ were assayed for β-galactosidase activity using previously described methods (Taylor and Heinrichs (2002) Mol. Microbiol. 43:1603-1614). Briefly, cultures were grown in TMS supplemented with 1 μM EDDHA or FeCl₃ to an O.D.₆₀₀=0.8. Cells (5×10⁸) were lysed in 10 mM potassium phosphate buffer (pH 7.8), 15 mM EDTA, 1% Triton X-100 and 10 μg lysostaphin at 37° C. After centrifugation of cell debris, 5 μl of supernatant were assayed for β-galactosidase activity using the Galacto-Light Plus Chemiluminescent reporter gene kit (Tropix) in a Berthold luminometer. The background was set at 50 RLU/s and the data presented are mean rlu/s of three independent samples, ±standard error.

### Siderophore Production Assays and Isolation of Siderophore

Siderophore activity in spent culture supernatants was assayed using chrome azurol S (CAS) by procedures previously described (Schwyn and Neilands (1987) Anal. Biochem. 160:47-56). Dilutions of culture supernatants were mixed with equal volumes of CAS shuttle solution and allowed to interact for 30 min at room temperature. With TMS medium serving as the blank, and DESFERAL® as reference standard, the absorbance at 630 nm was determined. Siderophore units were calculated using equation 1.

$$\frac{{A}_{630} ( TMS ) - {A}_{630} ( SAMPLE )}{{A}_{630} ( TMS )} \times 100 \% \qquad ( 1 )$$

For siderophore isolations, S. aureus strains were vigorously shaken in TMS for 48 h at 37° C. Culture supernatants were recovered by centrifugation and lyophilized. The concentrated supernatant was resuspended in 100% methanol to one-tenth the volume of the original culture supernatant and passed through Whatman No. 1 filter paper to remove particulate material. Rotary evaporation was used to reduce the volume before application to an LH-20 column (Amersham Biosciences). Fractions were collected and those testing positive with CAS shuttle solution and for biological activity in siderophore plate bioassays were dried, resuspended in water and examined by HPLC. Analytical reversed phased HPLC was used for final purification of siderophore. The column utilized was a 4.6×150 mm Waters ODS2 Spherisorb. 0.1% trifluoroacetic, acid, (TFA) in water represented solvent A, whereas 0.1% TFA in acetonitrile was used as solvent B. The chromatographic method used was as follows: at a flow rate of 0.75 ml/min, 6% B for 3.5 min, followed by a gradient of 6-60% B over 20 min. Staphylobactin was detected at 210 nm and had a retention time of approximately 17 min. Staphylobactin was collected, dried, and rechromatographed to check for purity and activity before being analyzed by ESI-MS.

### Electrospray Ionization-Mass Spectrometry (ESI-MS)

Electrospray ionization-MS and MS/MS analyses were performed on a Micromass quadrupole-time-of-flight (Q-TOF2) mass spectrometer fitted with a Z-spray source (Micromass, Manchester, UK). The detector was calibrated using an MS/MS spectrum of [Glu]-fibrinopeptide-B. The molecular mass of the siderophore sample was determined by flow injection analysis using a Waters CapLC system with a carrier solvent of 1:1 HPLC Grade methanol: HPLC Grade water at a flow rate of 30 μL/min. Spectra were acquired in positive ion mode with an m/z range of 50 to 1800 using the following parameters: capillary voltage, 3.2 kV; cone voltage, 30-40 V; desolvation temperature, 200° C.; source temperature, 80° C. Tandem mass spectra were acquired on the parent ion of interest using argon as the collision gas and collision energies ranging from 10 to 30 eV. All spectra were acquired and processed using MassLynx 3.5 (Micromass).

### Siderophore Plate Bioassays

The ability of siderophores to promote the iron-restricted growth of S. aureus was assessed using siderophore plate bioassays, performed as previously described (Sebulsky et al., (2000) J. Bacteriol. 182:4394-4400). Briefly, S. aureus RN6390 was incorporated into solid TMS medium (1.4×10⁴ cells/ml) containing 20 μM EDDHA. The ability of purified siderophores to promote growth of S. aureus was assessed after incubation of plates for 36 hours at 37° C.

### Mouse Kidney Abscess Experiments

Female Swiss-Webster mice, weighing 25 g, were purchased from Charles River Laboratories Canada, Inc., and housed in microisolator cages. Bacteria were grown overnight in TSB, harvested and washed three times in sterile saline. Pilot experiments demonstrated that S. aureus Newman colonized mice better in this model than did RN6390, and that the optimal amount of S. aureus Newman to inject into the tail vein to obtain an acute, but non-lethal kidney infection was 1×10⁷ CFU. Bacteria, suspended in sterile saline, were administered intravenously via the tail vein. The number of viable bacteria injected were confirmed by plating serial dilutions of the inoculum on TSB-agar containing 7.5% NaCl. On days five and six postinjection, mice were sacrificed and kidneys were aseptically removed. Using a PowerGen 700 Homogenizer, kidneys were homogenized for 45 seconds in sterile PBS containing 0.1% Triton X-100 and homogenate dilutions were plated on TSB-agar supplemented with 7.5% NaCl to enumerate recovered bacteria. Data presented are the log CFU recovered per mouse.

### Computer Analyses

DNA sequence analysis, oligonucleotide primer design and nucleotide sequence alignments were performed using the Vector NTI Suite software package (Informax Inc., Bethesda, Md.).

### Example 2

### S. aureus RN6390 and Newman produce siderophore

Herein we characterized the role that siderophore production plays in the iron-restricted growth of S. aureus in culture; we also examined its importance to in vivo growth and pathogenicity of this bacterium. To accomplish this, we generated genetically-defined siderophore-deficient mutants from siderophore-producing strains of S. aureus.

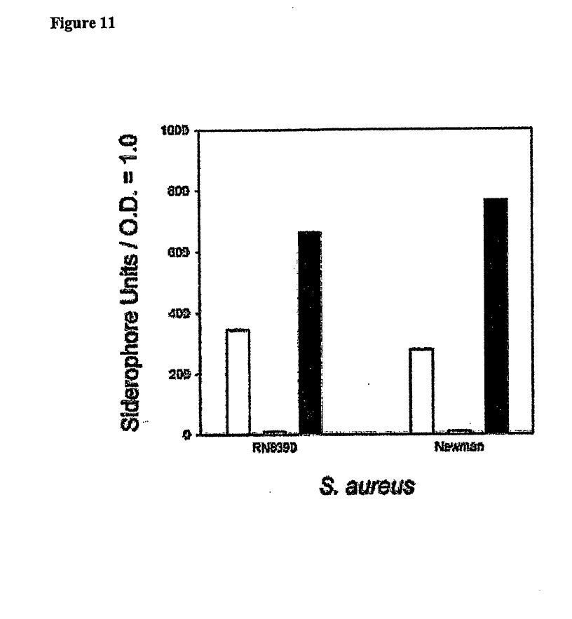

Previous studies have shown that various different isolates of S. aureus have the potential to produce multiple siderophores, including staphyloferrin A and staphyloferrin B (Meiwes et al., (1990) FEMS Microbiol. Lett. 67:201-206) and that the genetically-characterized strain 8325-4 produced siderophore(s), but of undetermined identity (Heinrichs et al. (1999) J. Bacteriol. 181:1436-1443; Horsburgh et al., (2001) J. Bacteriol. 183:468-475). We have demonstrated that two additional S. aureus strains that are used in our laboratory, strain RN6390 and strain Newman, produce readily detectable quantities of siderophore activity when the cells are grown under conditions of iron starvation, but produce very little siderophore during growth in iron-replete medium (FIG. 11). Noting that high-affinity iron acquisition systems, including siderophore production and iron(III)-siderophore uptake, are typically regulated by Fur in many different bacteria, we further showed that, indeed, in strains RN6390 and Newman, siderophore production was regulated by exogenous iron concentrations via the Fur protein, since fur derivatives of both RN6390 (H295) and Newman (H706) produced high levels of siderophore activity even when grown in iron-replete medium (FIG. 11). These findings are consistent with published results of Horsburgh et al. using S. aureus 8325-4 (Horsburgh et al., (2001) J. Bacteriol. 183:468-475).

### Example 3

### Isolation of Siderophore from S. aureus

Further, we wanted to identify which siderophore(s) was produced by S. aureus RN6390 and related strains. Given that siderophore production was derepressed in fur backgrounds, we isolated siderophore from culture supernatants of strain 11295 (RN6390 fur::Km). Our initial experiments focused on the isolation of staphyloferrin A and staphyloferrin B using published procedures (Haag et al. (1994) FEMS Microbiol. Lett. 115:125-130; Meiwes et al. (1990) FEMS Microbiol. Lett. 67:201-206). However, these purifications yielded extremely little CAS-positive material, suggesting that strain RN6390 produces no, or extremely little, staphyloferrin A or staphyloferrin B. Extraction of culture supernatants using a procedure that has previously been used to isolate ornibactins (Sokol et al., (1999) Infect Immun. 67:4443-55) did, however, result in the isolation of significant quantities of CAS-positive material. Chromatography of methanol-extracted culture supernatant through an LH-20 column yielded discrete fractions that were both CAS-positive and promoted the iron-restricted growth of S. aureus in siderophore plate bioassays. Further purification by reversed phase HPLC yielded an isolated peak of material that retained biological activity. Electrospray ionization-mass spectrometry (ESI-MS) analysis of the isolated material showed that it contained an abundance of a molecule with an m/z 822, which is significantly greater than that of previously characterized staphylococcal siderophores (staphyloferrin A m/z=480; staphyloferrin B m/z=448). We were unable to detect the presence of compounds in the active LH-20 fractions that matched the masses of either staphyloferrin A or staphyloferrin B. Taken together, these results strongly suggest that we have isolated a siderophore that has not previously been identified in the staphylococci. This siderophore is referred to herein as staphylobactin and efforts are ongoing to elucidate the structure of the molecule. Regarding the structure of the siderophore, one possibility is that one of the staphyloferrin molecules may comprise a part of the structure of staphylobactin.

### Example 4

### Identification and Analysis of a Siderophore Biosynthetic Gene Cluster in S. aureus

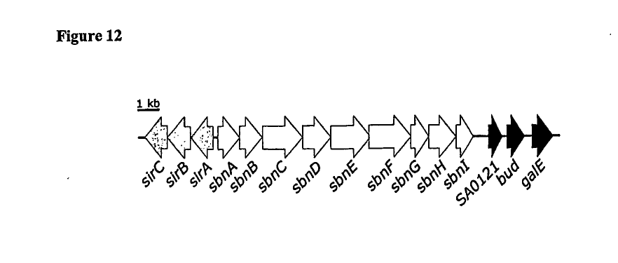

To resolve the genetic information underlying siderophore biosynthesis in the staphylococci, we searched S. aureus genome sequences from several strains and identified several open reading frames (orfs) whose products shared significant similarity with enzymes with demonstrated roles in siderophore biosynthesis. In particular, we identified an 11.5-kb gene cluster, situated between the sirABC operon and galE on the staphylococcal chromosome (FIG. 12), whose products share significant similarity with known or predicted siderophore biosynthetic enzymes in other bacteria (see Table 2). While the SirABC proteins share a high degree of similarity to iron(III)-siderophore transport proteins (Heinrichs et al., (1999) J. Bacteriol. 181:1436-1443), galE (encoding UDP-galactose-4-epimerase) is involved in nucleotide-sugar precursor formation. Hypothesizing that the 11.5-kb gene cluster was involved in siderophore biosynthesis, we designated the coding regions sbn, for siderophore biosynthesis.

To confirm that the sbn gene cluster was involved in siderophore biosynthesis in S. aureus, we insertionally-inactivated the fifth open reading frame (sbnE) with a kanamycin resistance cassette in S. aureus RN6390, thus creating strain H672. Methanol extracts of spent culture supernatant from iron-restricted H672 contained no trace of material that promoted S. aureus growth in siderophore plate bioassays. Biologically active siderophore was, however, consistently isolated from methanol extracts of iron-restricted supernatants of both the wildtype strain (RN6390) and strain H672 complemented with pSED32, a plasmid carrying sbnE, where expression of sbnE was driven by the plat promoter present on the vector. The staphylobactin molecule isolated from iron-restricted wild-type cultures was completely absent in iron-restricted supernatants of H672 and H675 (RN6390 fur sbnE). These results implicated sbnE as a key gene involved in the production of a siderophore and, more specifically, staphylobactin. The sbnE::km mutation was also transduced into S. aureus Newman, to create strain H686. Whereas staphylobactin was undetectable in supernatants of iron-starved H686, it was readily detectable in culture supernatants of iron-starved Newman. These results were confirmed by ESI-MS.

### Example 5

### The sbnABCDEFGHI Genes Comprise an Operon and Iron, via Fur, Regulates its Transcription

Predicted coding regions of the first nine open reading frames of the sbn locus either overlap or have very short non-coding segments separating them from one another, whereas approximately 600 by exist between the 3′ end of the ninth coding region and the 5′ end of the tenth coding region. This suggested that the operon may be comprised of nine open reading frames. The tenth coding region encodes a predicted protein of unknown function, the product of the eleventh coding region displays significant similarity to butanediol dehydrogenases (acetoin reductases) and the twelfth coding region is galE, encoding UDP-galactose-4-epimerase, which is involved in sugar-nucleotide precursor formation in polysaccharide biosynthesis.

In an effort to characterize the transcriptional regulation of the sbn operon, and to delineate the limits of the operon, targeted chromosomal lacZ reporter gene fusions were created to several coding regions, both within and beyond the putative sbn operon. β-galactosidase expression was then followed in strains bearing lacZ fusions when the cells were grown in either iron-replete or iron-deficient growth medium. When grown in the presence of 50 μM FeCl₃, expression of β-galactosidase in strains bearing fusions to sbnA, sbnF, sbnH and sbnI was at low, background levels whereas expression was well above background in strains bearing fusions to SA0121 and galE (Table 3). When grown in iron-deficient medium, however, all strains showed high levels of β-galactosidase expression. These results indicate that transcription of the sbn operon is iron-regulated through the ninth coding region (sbnI), and that expression of the tenth coding region and galE are not iron-regulated and likely play no role in the production of siderophore. The observation that sbnA was transcribed to the highest levels under iron-deficient growth conditions, while sbn genes further downstream appeared to be transcribed to lesser amounts under similar growth conditions, suggest that expression of the operon is controlled by one iron-regulated promoter element present upstream of the sbnA coding region.

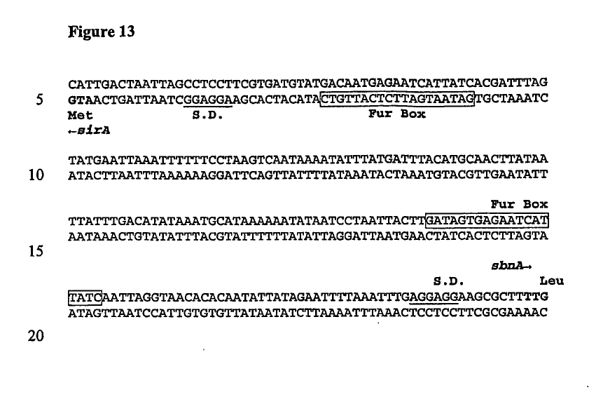

The putative sbnA start codon is preceded by a sequence which resembles a staphylococcal Shine-Dalgarno sequence (AGGAAGA) (FIG. 13) (Novick (1991) Genetic systems in staphylococci, p. 587-636. In J. H. Miller (ed.), Methods in Enzymology, vol. 204. Academic Press, Inc., San Diego, Calif.). Approximately 50 by further upstream, a 19-bp sequence (TGAGAATCATTATCAATTA) that bears a striking resemblance to consensus Fur boxes was found, suggesting that expression of the sbn operon is regulated by exogenous iron concentrations via the S. aureus Fur homolog. This would be consistent with our earlier observations (see above) that siderophore production was derepressed in a fur background. Indeed, in a fur-deficient background, β-galactosidase expression from the strain bearing a sbnF-lacZ fusion was extremely high when the cells were grown in iron-replete medium, indicating that the Fur protein represses transcription of the sbn operon under iron-rich growth conditions.

### Example 6

### An sbnE Mutant Demonstrates a Growth Defect in Iron-Deficient Medium

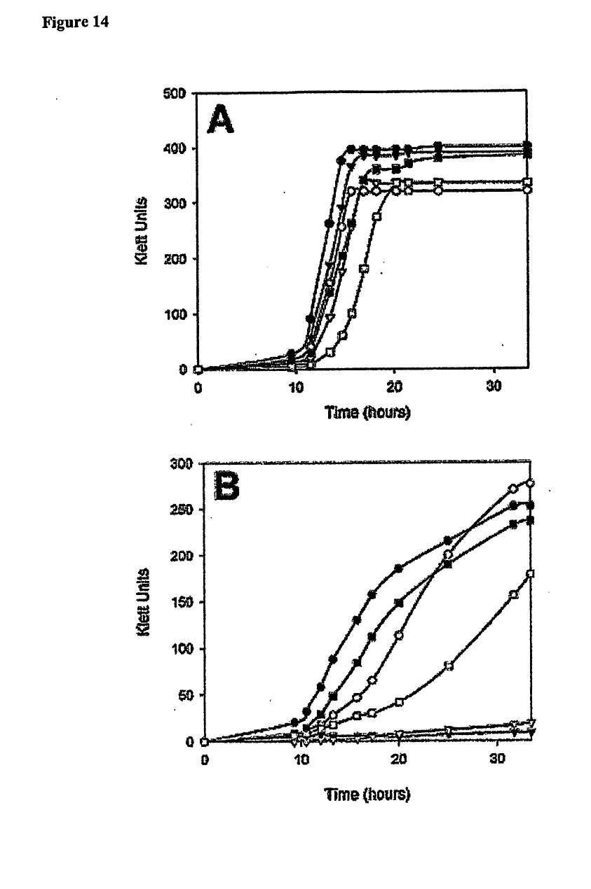

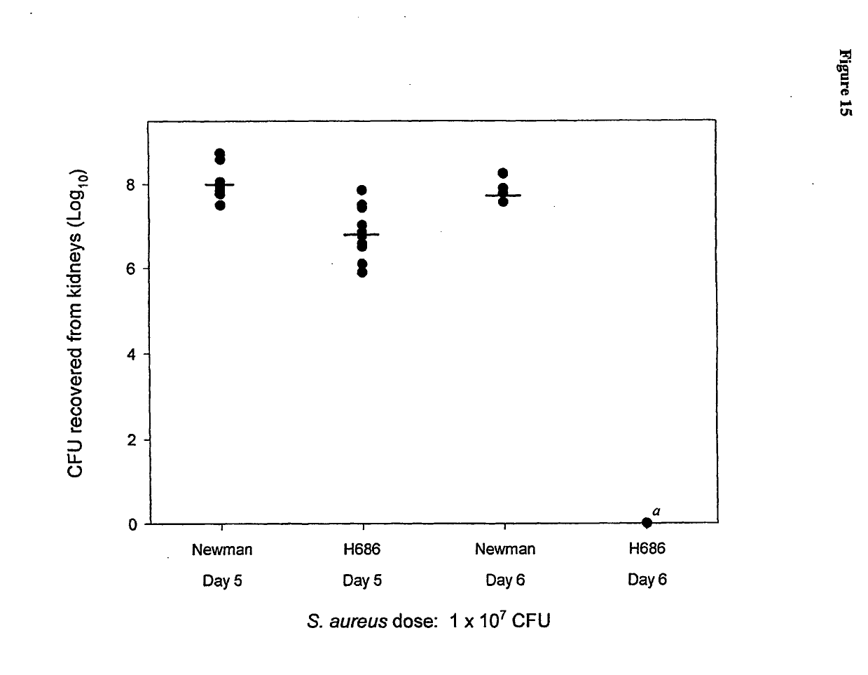

To assess the contribution of siderophore production to in vitro, growth of S. aureus, RN6390 and Newman, their isogenic sbnE::km mutants (H672 and H686, respectively), and the complemented mutants were grown in defined minimal medium. When grown in TMS medium supplemented with 10 μM EDDHA and 50 μM FeCl₃ (iron-replete medium), the growth yield of all of the strains was not appreciably different from one another (FIG. 14A). However, the growth of both H672 and H686 (sbnE mutants) was severely impaired, relative to their isogenic parents and the sbnE mutants carrying plasmid pSED32 (carrying multicopy sbnE gene), in the identical medium but lacking FeCl₃ (FIG. 14B). Given that the iron-sufficient versus the iron-deficient medium differed only by the presence or absence of FeCl₃, the suggestion that the poor growth phenotype of the sbnE mutants was due to the possible chelation of other essential elements by EDDHA can be ruled out. Thus, the sbnE mutants are impaired solely in iron acquisition.

While the sbnE mutant derivatives of RN6390 and Newman, H672 and H686 respectively, grew equivalent to their isogenic wildtype parents in iron-rich medium, the sbnE mutants were in contrast severely compromised in their ability to grow, relative to wildtype, under conditions of severe iron starvation (i.e., TMS supplemented with 10 μM EDDHA). We did observe, however, that at moderate levels of iron restriction (i.e., TMS supplemented with 1 μM EDDHA), H672 and H686 grew nearly as well as wildtype. The supernatants of mutants grown under these conditions did react positively in CAS assays, but we were unable to detect staphylobactin in culture supernatants. We also observed that S. aureus RN6390 grew significantly better under severe iron restriction than S. aureus Newman, and seemed to produce higher levels of siderophore activity as measured by CAS assays. In conclusion, we found that mutants in the sbn operon (e.g., sbnC::Km and sbnE::Km) do not produce staphylobactin and that all sbn genes are required for growth in serum. Further, the sbnE gene is dispensible for iron-replete growth, but is required for iron-restricted growth.

It is plausible that S. aureus RN6390 produces additional siderophore(s) that Newman lacks, and that they are produced under moderate levels of iron restriction. The significantly longer lag period of Newman versus RN6390 in growth assays under conditions of severe iron restriction (FIG. 14B) would support this argument. Alternatively, there may be differences in the regulation of staphylobactin production between the two strains. For example, the levels of iron restriction needed for expression of sbn genes or the amount of staphylobactin produced; may be different in Newman than in RN6390. Other research groups have reported differences in the levels of siderophore produced by different members of the staphylococci (Courcol et al. (1997) Infect. Immun: 65:1944-1948; Lindsay et al. (1994) Infect. Immun. 62:2309-2314).

### Example 7

### Siderophore Production Enhances the Virulence of S. aureus

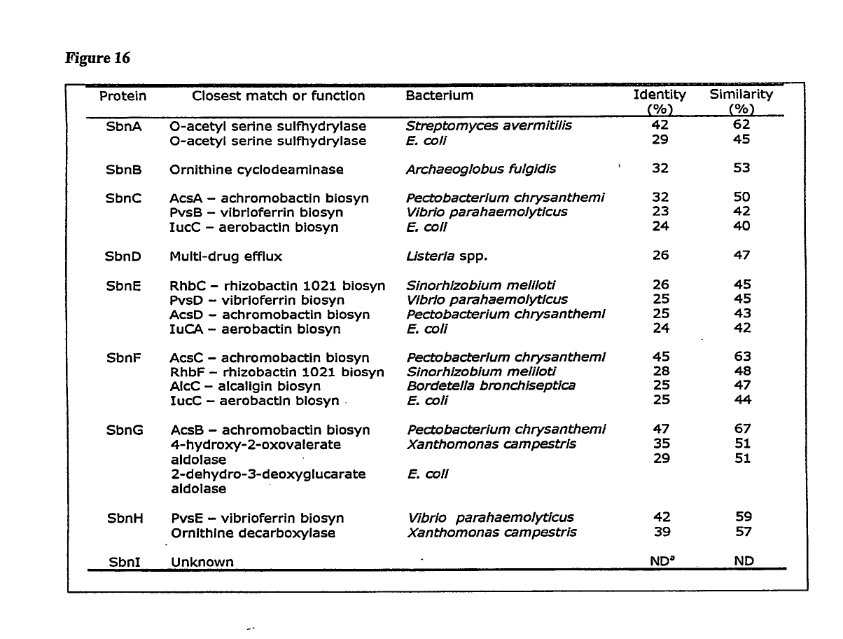

S. aureus can survive and replicate in blood to cause infection despite the fact that this environment is iron-restrictive. Moreover, recent reports have demonstrated that S. aureus can express proteins with the ability to bind to host iron sources such as heme and hemoglobin (Mazmanian et al. (2003) Science 299:906-9). Thus, in an effort to determine whether siderophore production in S. aureus is involved in the pathogenesis of this bacterium, the ability of the sbnE mutant to colonize mice was compared to that of its isogenic parent. Swiss-Webster mice were used in a murine kidney abscess model of S. aureus infection. On day 0, Swiss-Webster mice were injected with 10⁷ cfu of S. aureus via the tail vein. On days 2-3, we observed that the mice became ill and presented significant weight loss and lack of grooming. Between days 4-10, the mice became moribund and we commonly observed inflammation in the hinge quarters. The kidneys of individual mice injected with S. aureus Newman contained an average of greater than 1×10⁸ bacteria at both 5 and 6 days post-injection (FIG. 15). Kidneys from these mice possessed multiple cortical and medullar abscesses. In contrast, the kidneys from mice injected with H686 (Newman sbnE::km) lacked observable abscesses and average numbers of bacteria recovered from the kidneys were below 1×10⁷ at day 5 and no bacteria were recoverable at day 6 post-injection (FIG. 15), illustrating that the sbnE mutant bacteria were significantly attenuated in this model. The sbnE mutant is less lethal in a murine abscess model. Thus, these data implicate siderophore production as an important factor in the ability of S. aureus to survive in vivo.

### Example 8

### The sbn Operon is Present in S. aureus but not in the Coagulase-Negative Staphylococci

Given the demonstrated importance of siderophore production to the pathogenicity of S. aureus, we determined whether the sbn genes were specific to S. aureus or whether they were also present in other staphylococci. Dot blotting experiments, performed under low stringency hybridization conditions, were performed in efforts to detect sbnA, sbnC, sbnE and sbnH homologues in several other members of the staphylococci. Whereas sbn genes were readily detected in all laboratory and clinical strains of S. aureus tested (see Table 1 for a complete list of strains used), we were unable to detect the presence of these genes in any of thirteen different species of coagulase-negative staphylococci (see Table 1). Homologs of these genes are also not present in the genome sequences of S. epidermidis ATCC 12228 or RP62A. Since a previous investigation demonstrated the presence of the staphyloferrins in S. epidermidis strains (Meiwes et al. (1990) FEMS Microbiol. Lett. 67:201-206), this lends further support to the idea that the sbn operon is responsible for the production of a siderophore not previously identified in the staphylococci. Thus, the sbn operon appears to be specific to S. aureus among the staphylococci.

Further, our results suggest that the CoNS, generally less pathogenic than S. aureus due in large part to a relative lack of virulence factors, would appear to lack the ability to produce staphylobactin. As noted herein, the ability to produce this siderophore, synthesized via expression of the sbn operon, correlates with enhanced virulence of S. aureus in a murine kidney abscess model and may, therefore, represent another key determinant that dictates differences in the virulence of CoNS versus S. aureus.

### Example 9

### The sbn Operon is found in Ralstonia solanacearum

Interestingly, searches of the databases did reveal a similarly sized operon, present on a megaplasmid in the completed genome sequence of the phytopathogen Ralstonia (formerly Pseudomonas) solanacearum, whose products bear striking similarity to Sbn proteins (see Table 4). Indeed, it is highly likely that the two operons evolved from the same ancestor since the Ralstonia homologs are present in the same order as the sbn genes in S. aureus. The sbnE homolog in Ralstonia, however, is present on the complementary strand compared with the rest of the coding regions in the Ralstonia operon. Another minor difference between the regions in S. aureus and R. solanacearum is that the R. solanacearum sbnC and sbnD homologs appear to be fused into one coding region. A striking dissimilarity between the sbn operon in S. aureus and the homologous region of DNA in R. solanacearum is the mol % G+C of the respective operons. Whereas the operon in R. solanacearum has a mol % G+C of 72, the S. aureus sbn operon has a mol % G+C of 37. The mol % G+C of the S. aureus genome is approximately 32%.

### Example 10

### Sbn Mutant Phenotypes

The functions of the sbn proteins are presented in FIG. 16.

SbnA encodes a putative cysteine synthase, specifically an O-acetyl-L-serine sulfhydrylase. SbnA is thus likely involved in the conversion of L-serine (or O-acetyl-L-serine) to L-2,3-diaminopropionic acid and may work in conjunction with the activity of SbnB. A lacZ fusion to the sbnA gene was created and used to demonstrate that the sbnA gene is iron-regulated.

SbnB encodes a putative ornithine cyclodeaminase and may work in concert with SbnA to produce L-2,3-diaminopropionic acid, a likely precursor for staphylobactin. Ornithine cyclodeaminases mediate the deamination of ornithine and cyclization to proline and depended on NAD+. A mutation in sbnB was created by insertion of a Tet cassette. The sbnB mutant was compromised for growth in iron-restricted media and did not make staphylobactin. We also observed that the addition of proline does not bypass the sbnB mutation, suggesting that proline may not be the desired product required for staphylobactin synthesis. While proline is unlikely to be a siderophore precursor, ammonia may be a desired product for staphylobactin biosynthesis. In particular, SbnA and SbnB may produce diaminopropionic acid, which is a precursor of Staphyloferrin B. We observed that the iron-restricted phenotype of the sbnB::Tet mutant can be overcome by adding diaminopropionic acid to serum. Further, we observed in a mouse kidney abscess experiment, sbnB deficient strains were compromised for virulence (data not shown, n=7 mice).

SbnC encodes a putative IucC homolog for aerobactin biosynthesis (which performs the final condensation reaction in aerobactin biosynthesis). A mutation in sbnC was created by insertion of a Km cassette. The sbnC mutant displayed a similar growth phenotype as observed for the sbnB mutant in iron-restricted media. Further, the sbnC mutant does not produce staphylobactin.

SbnD encodes a putative multi-drug efflux pump. A mutation in sbnD was created by insertion of a Km cassette. The sbnD mutant displayed the same, growth phenotypes as the sbnB and sbnC mutants in iron-restricted media. No difference in MIC (minimum inhibitory concentration) values was observed for this strain and wild type strains against nalidixic acid, tetracycline, ethidium bromide and norfloxacin.

SbnE encodes a putative IucA homolog for aerobactin biosynthesis.

SbnF encodes a putative IucC homolog for aerobactin biosynthesis. A lacZ fusion to the sbnF gene was created and used to demonstrate that the sbnF gene is iron-regulated.

SbnG encodes a putative adolase.

SbnH encodes a putative ornithine or diaminopimelate decarboxylase. A mutation in sbnH was created by insertion of a Tet cassette and the mutant was compromised for growth in iron-restricted media. Further, a fusion of the sbnH gene to lacZ was made and this fusion was used to demonstrate that the sbnH gene is iron-regulated. While SbnI does not show homology to any proteins in the public databases, a lacZ fusion to the sbnI gene shows that the gene is iron-regulated.

### Example 11

### Biochemical Assays

Assays to screen for agents that disrupt the biochemical activity of SbnA, SbnB and SbnH in S. aureus will be conducted as follows. SbnB converts L-ornithine to L-proline and this reaction can be monitored by two methods. One is monitoring the conversion of NAD+ to NADH using a spectrophotometric assay for the reduction of NAD+. The second is using an HPLC-based assay to monitor the conversion of L-ornithine to L-proline. This reaction occurs early in the biosynthesis of staphylobactin. In another assay, SbnA activity is monitored by an HPLC-based assay. SbnA converts O-acetyl-L-serine to L-2,3-diaminopropionic acid. The reaction product is again monitored by HPLC-based methods. The reaction requires the participation of SbnB since the amine group provided by the L-ornithine is used during the conversion of O-acetyl-L-serine to L-2,3-diaminopropionic acid. SbnH activity can also be measured using HPLC. This enzyme likely converts L-ornithine into putrescine. Screening for inhibitors will entail screening for those compounds that result in the abolishment of the reaction end products.

### Example 12

### Expression Assays

Assays to screen for agents that disrupt the expression of SbnA in S. aureus will be conducted as follows. Wild type S. aureus cells will be cultured overnight in tryptic soy broth (TSB) (Difco) in the presence or absence of a test agent. Following 24 hours of culture, the cells will be washed in 1×PBS (phosphate buffered saline) and then lysed at 37° C. using 10 μg of lysostaphin in STE (0.1 M NaCl, 10 mM Tris-HCl [pH 8.0], 1 mM EDTA [pH 8.0]). The cell lysates will then be transferred to anti-SbnA antibody precoated plates and incubated for 45 to 60 minutes at room temperature. As a control, cell lysates from untreated S. aureus cells will be used. After three washes with water, a secondary antibody conjugated to either alkaline phosphatase (AP) or horseradish peroxidase (HRP) will be added and incubated for one hour. The plate will then be washed to separate the bound from the free antibody complex. A chemiluminescent substrate (alkaline phosphatase or Super Signal luminol solution from Pierce for horseradish peroxidase) will be used to detect bound antibody. A microplate luminometer will be used to detect the chemiluminescent signal. The absence of the signal in samples of cell lysates obtained from cells treated with test agent will indicate that the test agent inhibits the expression of SbnA. Similar expression assays may also be conducted for SbnB, SbnC, SbnD, SbnE, SbnF, SbnG, SbnH, SbnI and/or staphylobactin.

The practice of the present invention will employ, unless otherwise indicated, conventional techniques of cell biology, cell culture, molecular biology, transgenic biology, microbiology, recombinant DNA, and immunology, which are within the skill of the art. Such techniques are described in the literature. See, for example, Molecular Cloning: A Laboratory Manual, 2^{nd} Ed., ed. by Sambrook, Fritsch and Maniatis (Cold Spring Harbor Laboratory Press: 1989); DNA Cloning, Volumes I and II (D. N. Glover ed., 1985); Oligonucleotide Synthesis (M. J. Gait ed., 1984); Mullis et al. U.S. Pat. No. 4,683,195; Nucleic Acid Hybridization (B. D. Hames & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.), Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); Antibodies: A Laboratory Manual, and Animal Cell Culture (R. Freshney, ed. (1987)), Manipulating the Mouse Embryo, (Cold Spring Harbor Laboratory Press, Cold Spring Harbor, N.Y., 1986).

### INCORPORATION BY REFERENCE

All publications and patents mentioned herein are hereby incorporated by reference in their entirety as if each individual publication or patent was specifically and individually indicated to be incorporated by reference. In case of conflict; the present application, including any definitions herein, will control.

### EQUIVALENTS

Those skilled in the art will recognize, or be able to ascertain using no more than routine experimentation, many equivalents to the specific embodiments of the invention described herein. Such equivalents are intended to be encompassed by the following claims.

TABLE 1
Bacterial strains, plasmids and oligonucleotides used in this study

| Bacterial strain | | Source or |
|---|---|---|
| or plasmid | Description^{a} | reference |
| Bacteria | | |
| E. coli | | |
| DH5α | φ80dlacZΔM15 recA1 endA1 gyrA96 thi-1 hsdR17 | Promega |
| | (r_{κ}⁻m_{κ}⁺) supE44 relA1 deoR Δ(lacZYA-argF)U169 | |
| S. aureus | | |
| RN4220 | r_{κ}⁻m_{κ}⁺ | Kreiswirth et |
| | | al.^{c} |
| RN6390 | Prophage-cured wild-type strain | Peng et al.^{d} |
| Newman | Wild-type strain | O. Schneewind |
| SA113 | | T. Foster |
| ATCC 25923 | | ATCC |
| S. aureus MJH010 | 8325-4 fur::Tet; Tet^{r} | S. Foster |
| S. aureus H295 | RN6390 fur::Km; Km^{r} | Sebulsky et al.^{e} |
| H706 | Newman fur::Km; The fur::km marker from H295 | This study |
| | was transduced into Newman; Km^{r} | |
| H438 | RN4220 sbnF::pMUTIN4; Em^{r} | This study |
| H479 | H295 sbnF::pMUTIN4; Em^{r} Km^{r} | This study |
| H520 | RN4220 SA0121::pMUTIN4; Em^{r} | This study |
| H521 | RN4220 galE::pMUTIN4; Em^{r} | This study |
| H551 | RN4220 sbnI::pMUTIN4; Em^{r} | This study |
| H557 | RN4220 sbnH::pMUTIN4; Em^{r} | This study |
| H572 | RN4220 sbnA:: pMUTIN4; Em^{r} | This study |
| H672 | RN6390 sbnE::Km; Km^{r} | This study |
| H675 | RN6390 sbnE::Km fur::Tet; Km^{r}Tet^{r} | This study |
| H686 | Newman sbnE::Km; Km^{r} | This study |
| S. aureus H16 | Clinical isolate | LHSC |
| S. aureus H50 | Clinical isolate | LHSC |
| S. aureus H51 | Clinical isolate | LHSC |
| Coagulase-negative | | |
| staphylococci (CoNS) | | |
| S. auricularis ATCC 33753 | | ATCC |
| S. capitis ATCC 35661 | | ATCC |
| S. caprae ATCC 35538 | | ATCC |
| S. chromogenes ATCC 43764 | | ATCC |
| S. cohnii ATCC 29973 | | ATCC |
| S. epidermidis LK819 | | M. Valvano |
| S. haemolyticus ATCC 29970 | | ATCC |
| S. intermedius ATCC 29663 | | ATCC |
| S. hominis ATCC 27846 | | ATCC |
| S. sciuri ATCC 29062 | | ATCC |
| S. simulans ATCC 27851 | | ATCC |
| S. warneri ATCC 27836 | | ATCC |
| S. xylosus ATCC 35663 | | ATCC |
| Burkholderia cepacia | Genomovar III isolate from cystic fibrosis | M. Valvano |
| CEP024 | patient | |
| Plasmids | | |
| pAUL-A | Temperature-sensitive S. aureus suicide | Chakraborty et |
| | vector; Em^{r} Lc^{r} | al.^{f} |
| pAW8 | E. coli-S. aureus shuttle vector, Tet^{r} | Wada et al.^{g} |
| pBC SK(+) | E. coli cloning vector, Cm^{r} | Stratagene |
| pDG782 | pMLT22 derivative that carries a kanamycin | Guerout-Fleury |
| | resistance cassette; Ap^{r} Km^{r} | et al.^{h} |
| pMUTIN4 | lacZ fusion vector; Ap^{r} (E. coli), Em^{r} | Vanger et al.ⁱ |
| | (S. aureus) | |
| pSED12 | pBC SK+ derivative carrying sbnE; Cm^{r} | This study |
| pSED17 | pSED12 derivative containing sbnE::Km; Cm^{r}, Km^{r} | This study |
| pSED18 | pAUL-A derivative containing sbnE::Km; Km^{r}Em^{r} | This study |
| pSED32 | pAW8 derivative carrying sbnE; Tet^{r} | This study |

| | |
|---|---|
| Oligonucleotides | |
| Description | Sequence (5′ to 3′)^{b} |
| Generation of sbnA-lacZ | TTGGATCCAGTATATGAATCCTGGAGGC (forward), |
| fusion | TTGGATCCAAAAATGACTGACCCTTTCGCATC (reverse) |
| Generation of sbnF-lacZ | TGGATCCCATCACCAATTGAGCGTGTCGTAGGAGAT (forward), |
| fusion | TGGATCCTTTCAATTGTATGAGGCGCCAACACTCGT (reverse) |
| Generation of sbnH-lacZ | TTGCGGCCGCGATAGATAGAGATATCATTA (forward), |
| fusion | TTGGATCCTAGTTAACGCCTATGCCACC (reverse) |
| Generation of sbnI-lacZ | TTGCGGCCGCCCCAACACAATTTGGTATTTCTGAA (forward), |
| fusion | TTGGATCCTACTTGAAAATGTGCTTCGC (reverse) |
| Generation of SA0121-lacZ | TTGCGGCCGCAAGTTCCATTTGGTGTGTGG (forward), |
| fusion | TTGGATCCGGTAAACAGTGAAAAGAGC (reverse) |
| Generation of galE-lacZ | TTGCGGCCGCTATTATCGCTTTAGTATTAT (forward), |
| fusion | TTGGATCCTCAACGCCTGCTTGAGATGTT (reverse) |
| Cloning of sbnE gene | TTGGATCCATTAGCAGACATAGATATAT (forward), |
| | TTGGATCCTAGTGTCTCATCATTAATCG (reverse) |

^{a}Ap^{r}, Cm^{r}, Km^{r}, Lc^{r}, Tet^{r}, resistance to ampicillin, chloramphenicol, kanamycin, lincomycin, and tetracycline, respectively. LHSC, London Health Sciences Centre.
^{b}Restriction sites for subsequent cloning of the PCR products are underlined.
^{c}Kreisworth et al. (1983) Nature 305: 680-685.
^{d}Peng et al. (1988) J. Bacteriol. 170: 4365-4372.
^{e}Sebulsky et al. (2000) J. Bacteriol. 182: 4394-4400.
^{f}Chakraborty et al. (1992) J. Bacteriol. 174: 568-574.
^{g}Wada and Watanbe (1998) J. Bacteriol. 180: 2759-2765.
^{h}Guerout-Fleury et al. (1995) Gene 167: 335-336.
ⁱVagner et al. (1998) Microbiology 144: 3097-3104.

TABLE 2
Amino acid identity and similarity to proteins expressed from the sbn operon

| | | | Identity | Similarity |
|---|---|---|---|---|
| Protein | Closest match or function | Bacterium | (%) | (%) |
| SbnA | O-acetyl serine sulfhydrylase | Streptomyces avermitilis | 42 | 62 |
| | O-acetyl serine sulfhydrylase | E. coli | 29 | 45 |
| SbnB | Ornithine cyclodeaminase | Archaeoglobus fulgidis | 32 | 53 |
| SbnC | AcsA - achromobactin biosynthesis | Pectobacterium chrysanthemi | 32 | 50 |
| | PvsB - vibrioferrin biosynthesis | Vibrio parahaemolyticus | 23 | 42 |
| | IucC - aerobactin biosynthesis | E. coli | 24 | 40 |
| SbnD | Multi-drug efflux | Listeria spp. | 26 | 47 |
| SbnE | RhbC - rhizobactin 1021 biosynthesis | Sinorhizobium meliloti | 26 | 45 |
| | PvsD - vibrioferrin biosynthesis | Vibrio parahaemolyticus | 25 | 45 |
| | AcsD - achromobactin biosynthesis | Pectobacterium chrysanthemi | 25 | 43 |
| | IuCA - aerobactin biosynthesis | E. coli | 24 | 42 |
| SbnF | AcsC - achromobactin biosynthesis | Pectobacterium chrysanthemi | 45 | 63 |
| | RhbF - rhizobactin 1021 biosynthesis | Sinorhizobium meliloti | 28 | 48 |
| | AlcC - alcaligin biosynthesis | Bordetella bronchiseptica | 25 | 47 |
| | IucC - aerobactin biosynthesis | E.. coli | 25 | 44 |
| SbnG | AcsB - achromobactin biosynthesis | Pectobacterium chrysanthemi | 47 | 67 |
| | 4-hydroxy-2-oxovalerate aldolase | Xanthomonas campestris | 35 | 51 |
| | 2-dehydro-3-deoxyglucarate aldolase | E. coli | 29 | 51 |
| SbnH | PvsE - vibrioferrin biosynthesis | Vibrio parahaemolyticus | 42 | 59 |
| | Diaminopimelate decarboxylase | Xanthomonas campestris | 39 | 57 |
| SbnI | Unknown | | ND^{a} | ND |

^{a}ND, not determined.

TABLE 3
β-galactosidase expression from sbn-lacZ fusions

| Bacterial strain | Fe | β-galactosidase activity (rlu/s) |
|---|---|---|
| RN4220 | + | 0 ± 0 |
| RN4220 | − | 0 ± 0 |
| RN4220 sbnA::pMUTIN4 | + | 0 ± 0 |
| RN4220 sbnA::pMUTIN4 | − | 144763 ± 6080 |
| RN4220 sbnF::pMUTIN4 | + | 0 ± 0 |
| RN4220 sbnF::pMUTIN4 | − | 193944 ± 3398 |
| RN4220 sbnH::pMUTIN4 | + | 0 ± 0 |
| RN4220 sbnH::pMUTIN4 | − | 4660 ± 209 |
| RN4220 sbnI::pMUTIN4 | + | 0 ± 0 |
| RN4220 sbnI::pMUTIN4 | − | 3330 ± 188 |
| RN4220 SA0121::pMUTIN4 | + | 106 ± 3 |
| RN4220 SA0121::pMUTIN4 | − | 89 ± 10 |
| RN4220 galE::pMUTIN4 | + | 3046 ± 525 |
| RN4220 galE::pMUTIN4 | − | 2146 ± 76 |
| RN4220 fur sbnF::pMUTIN4 | + | 264425 ± 6581 |
| RN4220 fur sbnF::pMUTIN4 | − | 231425 ± 5720 |

TABLE 4
A homolog of the sbn operon in Ralstonia solanacearum

R. solanacearum homolog

| | Identity^{a} | Similarity |
|---|---|---|
| Sbn Protein | (%) | (%) |
| SbnA | 56 | 75 |
| SbnB | 58 | 75 |
| SbnC | 29 | 44 |
| SbnD | 28 | 42 |
| SbnE | 32 | 52 |
| SbnF | 36 | 54 |
| SbnG | 42 | 59 |
| SbnH | 47 | 63 |
| SbnI | ND | ND |

^{a}Identity and similarities are between the predicted protein products

1-32. (canceled)

33. A method of treating a microbial infection in an organism comprising the step of inhibiting staphylobactin synthesis in the organism by inhibiting the expression or activity of at least one of the polypeptides selected from the group consisting of SbnA, SbnB, SbnC, SbnD, SbnE, SbnF, SbnG, SbnH and SbnI.

34. The method of claim 33, wherein staphylobactin synthesis is inhibited by inhibiting the conversion of L-ornithine to L-proline.

35. The method of claim 34, wherein SbnB is inhibited.

36. The method of claim 33, wherein staphylobactin synthesis is inhibited by inhibiting the conversion of O-acetyl-L-serine to L-2,3-diaminopropionic acid.

37. The method of claim 36, wherein SbnA is inhibited.

38. The method of claim 33, wherein the organism is a mammal.

39. The method of claim 33, wherein the microbial infection is caused by S. aureus.

40. The method of claim 33, wherein the organism is a plant.

41. The method of claim 40, wherein in the microbial infection is caused by Ralstonia solanacearum.

42. A method of inhibiting staphylobactin synthesis in S. aureus cells comprising inhibiting the expression or activity of at least one of the polypeptides selected from the group consisting of SbnA, SbnB, SbnC, SbnD, SbnE, SbnF, SbnG, SbnH and SbnI in said cells.

43. The method as defined in claim 42, wherein the expression of the polypeptide is inhibited by blocking nucleic acid encoding the polypeptide.

44. The method as defined in claim 42, wherein the activity of the polypeptide is inhibited.

45. The method as defined in claim 42, wherein the growth of S. aureus is inhibited in iron-restricted conditions.

46. An isolated polypeptide involved in S. aureus staphylobactin synthesis selected from the group consisting of SbnA, SbnB, SbnC, SbnD, SbnE, SbnF, SbnG, SbnH and SbnI.
